SEQUENCE LISTING (47 sequences)

SEQ ID NO: 1 - protein, 399 aa, Homo sapiens
MAEAMDLGKDPNGPTHSSTLFVRDDGSSMSFYVRPSPAKRRLSTLILHGGGTVCRVQEPGAVLLAQPGEALAEASGDFISTQYILDCVERNERLELEAYRLGPASAADTGSEAKPGALAEGAAEPEPQRHAGRIAFTDADDVAILTYVKENARSPSSVTGNALWKAMEKSSLTQHSWQSLKDRYLKHLRGQEHKYLLGDAPVSPSSQKLKRKAEEDPEAADSGEPQNKRTPDLPEEEYVKEEIQENEEAVKKMLVEATREFEEVVVDESPPDFEIHITMCDDDPPTPEEDSETQPDEEEEEEEEKVSQPEVGAAIKIIRQLMEKFNLDLSTVTQAFLKNSGELEATSAFLASGQRADGYPIWSRQDDIDLQKDDEDTREALVKKFGAQNVARRIEFRKK

SEQ ID NO: 2 - RNA, 2196 nt, Homo sapiens
  source 1..2196: mol_type = mRNA
actctcgatgcgccgcggctgctgctgcgcaggcccagtgctgcgcttcgcggcagaggcgtctgcggtgacagctcagtcagttgagctctgtgtgccaggcgctcgcgagggggtagctcttctagtagtgctcggcgtcagacatggcggaggcgatggatttgggcaaagaccccaacgggcccacccattcctcgactctgttcgtgagggacgacggcagctccatgtccttctacgtgcggcccagcccggccaagcgtcggctgtcgacgctcatcctgcacggcggcggcaccgtgtgccgagtgcaggagcccggggccgtgctgctggcccagcccggggaggcgctggccgaggcctcgggtgatttcatctccacgcagtacatcctggactgcgtggagcgcaacgagaggctggagctggaggcctatcggctgggccccgcctcggcggcggacaccggctcggaagcaaagcccggggccctggccgagggcgccgcggagccggagccgcagcggcacgccgggcggatcgccttcacggatgcggacgacgtagccatccttacctacgtgaaggaaaatgcccgctcgcccagctccgtcaccggtaacgccttgtggaaagcgatggagaagagctcgctcacgcagcactcgtggcagtccctgaaggaccgctacctcaagcacctgcggggccaggagcataagtacctgctgggggacgcgccggtgagcccctcctcccagaagctcaagcggaaggcggaggaggacccggaggccgcggatagcggggaaccacagaataagagaactccagatttgcctgaagaagagtatgtgaaggaagaaatccaggagaatgaagaagcagtcaaaaagatgcttgtggaagccacccgggagtttgaggaggttgtggtggatgagagccctcctgattttgaaatacatataactatgtgtgatgatgatccacccacacctgaggaagactcagaaacacagcctgatgaggaggaagaagaagaagaagaaaaagtttctcaaccagaggtgggagctgccattaagatcattcggcagttaatggagaagtttaacttggatctatcaacagttacacaggccttcctaaaaaatagtggtgagctggaggctacttccgccttcttagcgtctggtcagagagctgatggatatcccatttggtcccgacaagatgacatagatttgcaaaaagatgatgaggataccagagaggcattggtcaaaaaatttggtgctcagaatgtagctcggaggattgaatttcgaaagaaataattggcaagataatgagaaaagaaaaaagtcatggtaggtgaggtggttaaaaaaaattgtgaccaatgaactttagagagttcttgcattggaactggcacttattttctgaccatcgctgctgttgctctgtgagtcctagatttttgtagccaagcagagttgtagagggggataaaaagaaaagaaattggatgtatttacagctgtccttgaacaagtatcaatgtgtttatgaaaggaagatctaaatcagacaggagttggtctacatagtagtaatccattgttggaatggaacccttgctatagtagtgacaaagtgaaaggaaatttaggaggcataggccatttcaggcagcataagtaatctcctgtcctttggcagaagctcctttagattgggatagattccaaataaagaatctagaaataggagaagatttaattatgaggccttgaacacggattatccccaaacccttgtcatttcccccagtgagctctgatttctagactgctttgaaaatgctgtattcattttgctaacttagtatttgggtaccctgctctttggctgttctttttttggagcccttctcagtcaagtctgccggatgtctttctttacctacccctcagttttccttaaaacgcgcacacaactctagagagtgttaagaataatgttacttggttaatgtgttatttattgagtattgtttgtgctaagcattgtgttagatttaaaaaattagtggattgactccactttgttgtgttgttttcattgttgaaaataaatataactttgtattcgagtctcgtcaaaaaaaaaaaaaaaaaaaaa

SEQ ID NO: 3 - protein, 626 aa, Homo sapiens
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVINMDYGMQEKNPIDHVSFYCKTAPNRAIRITKNQVSQLLPEKFAEQLIRVYCKKVDRKSLYAARQYFVQWCADRNFTKPQDGDVIAPLITPQKKEWNDSTSVQNPTRLREASKSRVQLFKDDPM

SEQ ID NO: 4 - RNA, 3189 nt, Homo sapiens
  source 1..3189: mol_type = mRNA
attgcgcctgcgcagggagcccaaggcaagagccgctaggctgccctgcccgaagggctcaactgtcagtgagcctgcgcaggaggccaataggctgccaatactccttggactccccgccagggccctgctgtcagtgcgcctgcgcgcgggtccggcgccgaggttcttgactgctgtgccggacgccaggtgtagccatgcagcgagccgattccgagcagccctccaagcgtccccgttgcgatgacagcccgagaaccccctcaaacaccccttccgcagaggcagactggtccccgggcctggaactccatcccgactacaagacatggggtccggagcaggtgtgctccttcctcaggcgcggtggctttgaagagccggtgctgctgaagaacatccgagaaaatgaaatcacaggcgcattactgccttgtcttgatgagtctcgttttgaaaatcttggagtaagttccttgggggagaggaagaagctgcttagttatatccagcgattggttcaaatccacgttgatacaatgaaggtaattaatgatcctatccatggccacattgagctccaccctctcctcgtccgaatcattgatacacctcaatttcaacgtcttcgatacatcaaacagctgggaggtggttactatgtttttccaggagcttcacacaatcgatttgagcatagtctaggggtggggtatctagcaggatgtctagttcacgcactgggtgaaaaacaaccagagctgcagataagtgaacgagatgttctctgtgttcagattgctggactttgtcatgatctcggtcatgggccattttctcacatgtttgatggacgatttattccacttgctcgcccggaggtgaaatggacgcatgaacaaggctcagttatgatgtttgagcaccttattaattctaatggaattaagcctgtcatggaacaatatggtctcatccctgaagaagatatttgctttataaaggaacaaattgtaggaccacttgaatcacctgtcgaagattcattgtggccatataaagggcgtcctgaaaacaaaagcttcctttatgagatagtatctaataaaagaaatggcattgatgtggacaaatgggattattttgccagggactgccatcatcttggaatccaaaataattttgattacaagcgctttattaagtttgcccgtgtctgtgaagtagacaatgagttgcgtatttgtgctagagataaggaagttggaaatctgtatgacatgttccacactcgcaactctttacaccgtagagcttatcaacacaaagttggcaacattattgatacaatgattacagatgctttcctcaaagcagatgactacatagagattacaggtgctggaggaaaaaagtatcgcatttctacagcaattgacgacatggaagcctatactaagctgacagataacatttttctggagattttatactctactgatcccaaattgaaagacgcacgagagattttaaaacaaattgaataccgtaatctattcaagtatgtgggtgagacgcagccaacaggacaaataaagattaaaagggaggactatgaatctcttccaaaagaggttgccagtgctaaacccaaagtattgctagacgtgaaactgaaggctgaagattttatagtggatgttatcaacatggattatggaatgcaagaaaagaatccaattgatcatgttagcttctattgtaagactgcccccaacagagcaatcaggattactaaaaaccaggtttcacaacttctgccagagaaatttgcagagcagctgattcgagtatattgtaagaaggtggacagaaagagtttgtatgccgcaagacaatattttgttcagtggtgtgcagacagaaatttcaccaagccgcaggatggcgatgttatagccccactcataacacctcaaaaaaaggaatggaacgacagtacttcagtccaaaatccaactcgcctccgagaagcatccaaaagcagagtccagctttttaaagatgacccaatgtgaatgtctgtagtcagttgtttacaaactccctctcctgcacaattcatttagaggcttcaatcatagaattctgcaaattaatgacaactcatgctttaattttgtattttgaatgtacacgcatgctgaagctaagtaacttttaatcaaagaaataagatggtattaggcaaatcttactatactatgaaaagcattaccttgcctatttttaatattattaaagcctttctccttcagtagtctattttcttagaataacaactcttttatctattctgaactctattttttttcttttttaagagacaaggttttgctctgttgcccagcttggactcgaactttcctgggctcaagcgaccctcctgcctcagccccccaagtagctgggactaaagtcatgtgccaccacacccagcttactctgaacttttatgacagatgattgttttttgtttttaatgtagaaatgagacaagggtacaaattggaactagaaattgacattgtcattgacaaacatggctaaaaacaaaacatcaaatcctgcccccgtgaagagttccctgtcacctcaagtttgaggatagtcctctaagagtgacctaagcataagtgaaagacacctcccctcacccttctagccccctacaaggtgccaggttggggtaaagagttggagatgatggccaggagtggcctccaacacgctggtgagaggcctgattaggttttggggaagatctgagagctctggcctcttcgtgagtggaacataaagccgcctcttgttgggagatcctaccccagtgacagaggaatcccccaaactaggctgtgccctggctccgtggcggctccagacccgggtagtgcctttgtcccctgaatactcactcccccggtccagagggccttcccactgcccagcctggagaaggcctcccctgacctgctctctcagtatcctggagagctggccagaggccatcacaggcatcatcctcagagctcctcagacctgggactttgtttttgctggttcagtgcattttgtgtatttaagagcaaacactagccaggcgtggcggcgtgtgcctgtagt

SEQ ID NO: 5 - protein, 631 aa, Homo sapiens
MGLPRGPEGQGLPEVETREDEEQNVKLTEILELLVAAGYFRARIKGLSPFDKVVGGMTWCITTCNFDVDVDLLFQENSTIGQKIALSEKIVSVLPRMKCPHQLEPHQIQGMDFIHIFPVVQWLVKRAIETKEEMGDYIRSYSVSQFQKTYSLPEDDDFIKRKEKAIKTVVDLSEVYKPRRKYKRHQGAEELLDEESRIHATLLEYGRRYGFSRQSKMEKAEDKKTALPAGLSATEKADAHEEDELRAAEEQRIQSLMTKMTAMANEESRLTASSVGQIVGLCSAEIKQIVSEYAEKQSELSAEESPEKLGTSQLHRRKVISLNKQIAQKTKHLEELRASHTSLQARYNEAKKTLTELKTYSEKLDKEQAALEKIESKADPSILQNLRALVAMNENLKSQEQEFKAHCREEMTRLQQEIENLKAERAPRGDEKTLSSGEPPGTLTSAMTHDEDLDRRYNMEKEKLYKIRLLQARRNREIAILHRKIDEVPSRAELIQYQKRFIELYRQISAVHKETKQFFTLYNTLDDKKVYLEKEISLLNSIHENFSQAMASPAARDQFLRQMEQIVEGIKQSRMKMEKKKQENKMRRDQLNDQYLELLEKQRLYFKTVKEFKEEGRKNEMLLSKVKAKAS

SEQ ID NO: 6 - RNA, 6946 nt, Homo sapiens
  source 1..6946: mol_type = mRNA
atgctctgcccggctgcggcgcttcgggcaggcggcggcggcggcggcggcggcggcagagggagtttccgctttgtactccaccccggtagcagctccgcggcagggacagcttcctccggacgcttggcgggcttcgctctcgccttacgacagcccggtcggatcatggggttgcccagggggccggagggccagggtctcccggaggtggaaacaagagaagatgaagaacaaaatgtcaagttgactgaaattctggagctcttggttgcagctgggtatttcagggcaagaattaaaggcttatcaccctttgacaaggtagtaggaggaatgacttggtgtatcaccacttgcaactttgatgtagatgttgatttgctctttcaagaaaactctacgataggtcaaaaaatagctctgtcagaaaaaattgtctcggtcctgccaaggatgaaatgcccacaccagctggagccccaccagatccaggggatggattttattcacatatttcctgttgttcagtggctggtgaaacgagctatagaaacaaaagaagagatgggtgactatatccgctcctactctgtatcccagttccagaagacttacagtctccctgaggatgatgacttcataaagagaaaagaaaaggccatcaagacagttgtggacctctcagaagtgtacaagccccgtcggaaatacaaacgccaccagggagcagaggagctacttgatgaagaatctcgaatccatgctacacttttggaatatggcaggagatatggatttagccgccagagcaaaatggagaaggctgaggacaagaaaacggcacttccagcagggctgtcagctacagaaaaagctgatgcccacgaggaagatgagcttcgagcagctgaagagcagcgtattcagtcgctgatgaccaagatgaccgctatggcaaatgaggagagccgtctcaccgcaagctccgtgggccagattgtgggactctgctctgctgagatcaagcagattgtgtccgagtatgcagagaagcagtctgagctatcagctgaagaaagtccagaaaaattaggaacctcccagctacatcgccggaaagtcatttccttgaacaaacagattgcgcaaaagaccaaacatcttgaagagctgcgagcaagtcacaccagcctacaagccagatataatgaagccaagaaaacgctgacagagctgaagacttacagtgagaaactggacaaagagcaagcagccctcgagaagatagaatccaaagctgatccaagtatcctacagaacctgagagcacttgtagccatgaatgaaaatctgaaaagtcaagaacaggaatttaaagcacattgtcgagaggagatgacacgactacagcaagaaattgaaaacctgaaagctgagagagcaccacgtggagatgaaaagaccctctccagtggagagccgcctggtaccttgacctctgcaatgactcatgacgaagacctagacagacggtataatatggagaaagagaaactttacaagatacgtttactacaggctcgaagaaatcgagaaatagcaattttgcaccgcaagattgatgaagtccctagccgtgccgagctaatacagtatcagaagagatttattgaactctaccgccagatttcagcagtgcacaaagaaaccaagcagttcttcactttatataataccctggatgataaaaaggtttatttggaaaaagagattagtctgctgaactcaattcatgagaacttctcacaggccatggcctcccctgctgcccgggaccagtttttacgtcagatggaacagattgtggaaggaattaagcaaagtagaatgaagatggaaaagaaaaagcaagagaacaaaatgagaagagaccagttgaacgaccagtacttggagctgttagaaaagcagaggctatactttaagactgtgaaagagttcaaggaggagggccgcaagaacgagatgctgctgtccaaggtgaaagcgaaggcctcctgaacatccccagccgtggctgtatgtcattgattttacttttaagcaccgtatatcacctacaagatcatgaaatggttctgaaagcgacagtagagagatgcagttgtgatgatttcaacaacctggatgttttctttctcctctttgcttccattcatctctgttggctgctgttgatggagtcagacagtaaacacgtggcttggataacacccatcatcctatgaagaatatagggagtacttgttctctgttgattcaacttttatgtctccagtaacattgcgcttatgaaggtacctgtatttgtatggactctgaataaagaagaattcatttgtttagcaagtattagttcagcaaccactgagaaataagcactgaggaagattcagagacgtgtaaaacacagttcctactgcacaagtacccagcaggtggcccagggaggcagatacagcacacttgaccgcagaactgggctatccaagatgtttttcagtaaacagaaggcatttagctgaaatgatcagcccatgtagtgttggtcacttgggcctttcacctgccatggtaccttttgttcccagctcctccaggtgccagccagcaggcttggtggtgacagcaactggaacgaaagttcagtgttgttttaatttttatacgttactcaagttgatttctcagaaaattgaaaacagaccttgtgctgaggacacgtcaataaaaattataccttcccctgccccaatgttcatgtgagtcacagcatgcagaggggctgtgcatccccctcattgaaggcacatttgatgaggctacgctgcagatgggtcatggtctgagccaggagagagatagacactgttccctgcactcaaggcaagtagggtgtcaggaggaagagcagtagggtttgcagagatccagggaaacttcctggaggaggcagcatttgagccctatctaaagggagaatcggatgttgataggaagactgttttaaatgaaaatacactatgattaaaaacccaagagagtgcagaggagtacaaggcatgcatgttgggaaaggagggaataagctagtctctgcaggaggcttcagagacccgtctgggaagtagggctgagactaggctttggagggccttgaagggcagctgagggttgattttaatccttggaaatgaccagtgcagatcaaacatggagctcctgctgccctggtcctcaggagtctggtaactcagtcatgttctgtaggtttgtgctccattttgcataaatcttgcaaacataccaggtggcaggtttgcacagtaacacaaaagcttaaacatgtcaagcaagtggggcattcagcagccacagaaatgtgttcatttttagtccctaatggatgtgattaagcagggctgatgtgtacagatttaaaaacacacacacgtgctggattaaaaacagccactggtttcagtgccagggccagcaaggcctacctgagccaggcggaagcagcagcttgcaaatagaaactgccacctgcgagcgcttcttgggtcatgtgcagctccacctcagctggtccctgagccgtggtcatgtgggggttttgaagggctaagttgaagcaagtaattgcaaaagaacaggtggcactctgagggcaggggaaggagacagaagtttgactgcgtttgaaattatttcagtcagggaaacatctgcgttgtcttgttaagcagcttgtaggcccacccctggctaactgaactttgtgagcagttggggcagttatttgaatatttgtccaaaggtggtagggaggcagatgacattctggggttactggagtccacctctgtgctgatggggaaagtatggcatgatgaatccaaagcaaccagtaattctttttgaaagtgcaggcatttattttgagctgtatttcataggagtttcatttggtttggttttggtatagagaggtgtccatagatctctattccctttttttttttatcgacaaggcctactacggataaatcagtttttgttgcctgaatacactttgaattagactacagaagccagtttgagctcaaatttcacacttgctgcagacaactctcagtgtggacagtgcctttctgagtaattaggatgttacatgactttttggtgaagccgtatagggtaaggagatgccccaaggcccctgatcatttcagaagccacctggagcaccagaaggggtcatcttcccagggctatggctctgtgtgcttagacagacatagtatggaattgtgctctaagcccacaggcttctccggagttaaaaggaagcaagaacatccttaagttactttttcaaaatatgcgcttcaggaggaaccaggtttccccaggagagagaggctccctgctcaatcagcacagggagttgctagcgggcaggctgcccagtgctgtggaatctccgccagcctctgggttgggggctccctcccacttcttgcttcttatggagtacatgggactgctctgctttaagccgcgatcagagctgttgtgagcaggacaagattgcgtgatcacgaggtgcgtggggagatgctaggacatttgttgatgtctttgacctgtgctccgtctctgttggatgactgggttagctaacaccactagagggaggtacagaatgttaaacagtcgtcagagtcacactgagggttgttttaacagagggcaaattcccagtttgtcaagcatatctctgttagagtccgaagttcatggttttaatggttctagctcgtagcccttaatattttcaggatagctgggtcctgccagtgtccgctgagtccatcagcacaaccagaaggtccagggtccaggcatgggaagcattctctgacaataccgagccctcgggacctcctgaaaccttgagcagaggctggagaagaccttcagacagtccttttactacctcacaaagtgccatgcccgggacctcagacctcagggttgtgacgttgttgatgttgtcaggccccagtacgtctctagtgaccgaagcagacctgcctgaaagggcactccggacagtgagccctggctctaacctgtgccctcatatttctgctgcctcatatggtcttgtttgcccactctgtaggtgactgatcaccctaaaagggaggtaggtatctagcagtagcacagttactttgagcctgaccacttgttctgaagtgcagatgttgaaacatgcattgaggaagaacactttaaggaagcactttatgatttactcatggctggtgttggcctcaaagtaaaagaacatcttaatgcagaacagtacaataaagagccccataggatggatgccatcatctgtccccatggttgggattgccaaggaaagtgccttgttctgtgcatcttcagaatcccccaggccctctcttgatccttttcatgaagtccagagcattccttctattacaggctgaagatgcattgtaaaaggtaatgctataatagtattccctcttctcaataaatgtgcaaaactggagaatctggtgctgatctgcagcatacttgacaacacagggaataccgtggatgttccacgccagccttacatccgtccccattgcctgcccatgtccagtggccctgcgcaacaaaagtaagggcttgtcttaccacactctccccagcatctagctcaaggtagatgactgactgggaaattccggttggataaaatggcttttccacattgcaactttgcatctctattaggaaaatttagattgaacctttcctctacttgatgcttagtttgagaacagttttcacaactttagagggtctctctcagagtatgttatactgtaaccttgaagtttgagatgaaacaaggtgtcagaggttttgaatctttcaaaggaattgccttttacagatttttttttttattccctaagaagggaaaaagagagggaagaactacactaatgttagagataaggtatgttttggctcaaaatgtgtcatgaacctaaaggtaacttgatgggcagaaatgtctcagccatgtgaaccaagaagtgggaccacagtgcaaggctgtgggtggcagcactagatacggtggtggcaggagtgtgacatgtaggcactccatgggggacagctggacggtgtctatcaaaagtgcaaatgcataagctctttgaactagcaatgctactcctaagaattgtacctgtgtacaaggtacttgtgtacaagagtttttgttgaaacattgtaatagcaaaaagttggagagtgccaccaatacaggggaatggttaaatacattatggccagcttaaacaatggaatagtttgtagaaatttaaaacaatggatcctgtgtgtgtgtactaccatagaatgggccccaagatgcatgattaagtgaggaaaaaagcccatggtatcctaatgtttggaatggagaaagtgtgtagatgtatgtgtgtgcatatgttttcacacagacgtatacacgtgtatatcatatgtccacctgcatatgcctaggatgtctctgggaggatatctagcagcccggcagcctgaattgcctctgggaagaggggtgggaatgacttttcaatgtacaccttttgtaccttttgaattttgtaccatgtacatgtattatctattaaaaaatagttcttaatttttaatttggtatacttttcttaatagaaccaaaatggaagaaaattctgattctctaaatctgctttcttaaagataaagcctttgattcaaaagtaaaaaaaaaaaaaaaaaa

SEQ ID NO: 7 - protein, 926 aa, Homo sapiens
MSRRRKHDDSPSPKKTPHKTVAAEECGSVVEPGRRRLRSARGSWPCGAREGPPGPVRQREQPPTAALCSKSNPEERYETPKRALKMDSLSSSFSSPNDPDGQNDIFWDQNSPLTKQLGKGRKKQIYTTDSDEISHIVNRIAPQDEKPTTNSMLDMWIGETAIPCTPSVAKGKSRAKISCTKLKTQSQEEELMKLAKQFDKNMEELDVIQEQNKRNYDFTQMISETEILSNYKDNIQMWSLHNIVPEIDNATKKPIKGNTKISVANNQNSSQKPFDQIAEAAFNAIFDGSTQKCSGQLSQELPEAFWSTSNTTFVKTNALKEEKIITNETLVIEKLSNKTPRSLSSQVDTPIMTKSCVTSCTKEPETSNKYIDAFTTSDFEDDWENLLGSEPFAMQNIDMPELFPSKTAHVTDQKEICTFNSKTVKNTSRANTSPDARLGDSKVLQDLSSKTYDRELIDAEYRFSPNSNKSNKLSTGNKMKFENSSNKIVIQDEIQNCIVTSNLTKIKEDILTNSTEASERKSALNTRYSNEQKNKCILNQSIKAPVNTDLFGSANLGSKTSVSNPNQTSASKVGSFFDDWNDPSFANEIIKACHQLDNTWEADDVDDDLLYQACDDIERLTQQQDIRKDSKTSESICEINNNSEHGAKLTQQQDIRKDSKTSESICEINNNSEHGAKNMFAISKQGSNLVQSKHLNPGSISVQTSLTNSSQIDKPMKMEKGEMYGNSPRFLGATNLTMYSKISNCQINNLHVSYTNTDVPIQVNSSKLVLPGSSSLNVTSDHMNTEITTYKKKLSTNQPCHKTVTDEAQSNLNTTVGFSKFTFTRMKNSQILSQFNQNCITGSMSDTKITQGVEKKKGVNPLLEEAVGQQSLVKLSESLKQSSKEEEEKNRKCSPEEIQRKRQEALVRRMAKARASSVNAAPTSFL

SEQ ID NO: 8 - RNA, 3298 nt, Homo sapiens
  source 1..3298: mol_type = mRNA
cggccgcctcttgcgcgcgccccaccgaccaaaatggcggctgccgttggtgcggggtgcggtttgtagtgctgttgccctactcatccctttgcaaaatgtgaaagaagaagcggctggtggaggcgggccataggcaatgagtcggcgaaggaaacatgatgacagccctagcccgaagaaaacgccgcacaaaacagtggcggcggaggaatgcggctcggtggtcgagccagggaggaggcggctgagatcggcccgcggttcgtggccctgcggggctagagaggggcctcccgggccagtgcggcagcgagagcagcctccgaccgccgccctgtgcagtaaaagtaaccccgaggaaaggtatgaaacaccaaagagagcgctgaaaatggactcactgtcatcttccttcagttctcctaatgatccagatggacagaatgatatcttttgggatcagaattctccattgacaaagcagttaggtaaaggaagaaaaaaacagatttacaccacagatagtgatgagatttcacatattgttaatcgtattgctcctcaggatgaaaaaccaacaacaaattctatgctggacatgtggattggtgaaactgctattccttgtactcccagtgtagcaaaaggaaaatcaagagcaaaaatcagctgcacaaagttaaaaacacaaagtcaagaagaagaacttatgaaactggctaaacaatttgataaaaatatggaagagctagatgtgattcaagagcaaaacaagaggaattatgattttacccagatgatttcagaaacagagattttaagtaattataaagataatatacagatgtggtcattacataatatagttcccgaaatagataatgctacaaaaaagccaatcaaaggaaacaccaagatatctgtggcaaataatcaaaatagcagtcagaagccatttgaccaaattgctgaagcagcctttaatgctatttttgatggttctactcagaaatgtagcggacagttaagccaagaactgccagaggctttttggagcaccagtaatactacctttgtaaagacaaatgctttgaaagaggagaaaatcattactaatgaaactctggtcattgaaaaactgtcaaataaaaccccacgatcactttcttctcaagtagatacacccataatgacaaaatcatgtgtgacttcctgtactaaggagccagaaacttctaataagtacattgatgcatttactacaagtgattttgaggatgattgggaaaacttactaggtagtgaaccttttgctatgcaaaatatcgacatgcctgaactctttccttctaaaacagcccatgttactgatcaaaaggaaatttgtacctttaatagtaaaactgttaaaaatacgtcaagagcaaatacaagtccagatgccaggttaggagattcaaaagtattacaagatctttcttcaaagacatatgacagagaattaatagatgcagaatatagattttcaccaaattcaaataaatcaaacaaattatccactggaaataaaatgaaatttgagaactcttccaataaaattgttattcaagacgaaattcaaaattgtatagttacatctaatctgacaaaaataaaggaagatattcttactaactctactgaagcttctgaaaggaagtcagctttgaacacaaggtattctaatgaacagaaaaataagtgcattttaaatcagtctattaaagcccctgttaatactgatctttttggctctgcaaatctaggcagtaaaaccagtgttagtaacccaaatcagactagtgcatcaaaagtaggttctttctttgatgattggaatgatccctcatttgccaatgaaattattaaagcatgtcatcaattagataatacctgggaagcagatgatgtagatgatgatttgttgtaccaagcatgtgatgatattgaaagactaactcagcaacaagacattagaaaggacagtaagacatcagaaagtatatgtgagatcaataataattccgaacatggagccaaactaactcagcaacaagacattagaaaggacagtaagacatcagaaagtatatgtgagatcaataataattccgaacatggagccaaaaacatgtttgctatatctaaacaaggaagtaatttggtacaatcaaagcatttgaatccaggcagcatttcagtgcagacatctttgacaaatagctcacaaatagataagccaatgaagatggagaaaggggaaatgtatggaaattctccaagatttttaggtgccacaaatttgactatgtattctaagatctcaaactgtcagataaataatctgcatgtgtcttatactaacactgatgttccaatacaagtgaatagttccaaattggttcttccaggaagttcaagtttgaatgtaacttcagatcatatgaatacagaaattactacttataagaagaaattgagtactaatcagccatgccataagactgtaacagatgaagctcagagcaaccttaacacaacagttggattttcaaagtttacatttacaaggatgaaaaattctcagattctttctcagtttaatcaaaattgtataactggaagtatgtctgataccaaaattacacagggtgtggagaaaaagaaaggtgtcaacccattactggaggaagctgttggacagcaatctttggtgaaactttctgaatctttgaaacaatcttcaaaagaggaagaagagaaaaatagaaagtgttctcctgaagaaattcagagaaaaagacaagaagcactggttcggagaatggctaaagcacgagcctcatctgtaaatgcagctcccacttcatttctttaatgaaatattagttggaagacttcacgaagactgctgataactatctgtgattgataggaaattttttttcttgatttctctgtgagaaatgtaatgctgacttttataaagcctggacttctactttatttaataaatcaatgtttgcaatggtaaatgaaacatttccttggacatgtatttgaaagtcattaaatacaaaagttttggaaattcaggaaagttagcaattatgtacggatattatacagaggaaagtagttatatttttaaatgctattattgcagaggatcatcaaaaaagaggtaatctacgttatttcctattctaatgtcttttcctaataaaaaacttcaactttcaaaaaaaaaaaaaaa

SEQ ID NO: 9 - protein, 638 aa, Homo sapiens
MESCSVTRLECSGAISAHCSLHLPGSSDSPASASQIAGTTDAIWNEQEKAELFTDKFCQVCGVMLQFESQRISHYEGEKHAQNVSFYFQMHGEQNEVPGKKMKMHVENFQVHRYEGVDKNKFCDLCNMMFSSPLIAQSHYVGKVHAKKLKQLMEEHDQASPSGFQPEMAFSMRTYVCHICSIAFTSLDMFRSHMQGSEHQIKESIVINLVKNSRKTQDSYQNECADYINVQKARGLEAKTCFRKMEESSLETRRYREVVDSRPRHRMFEQRLPFETFRTYAAPYNISQAMEKQLPHSKKTYDSFQDELEDYIKVQKARGLDPKTCFRKMRENSVDTHGYREMVDSGPRSRMCEQRFSHEASQTYQRPYHISPVESQLPQWLPTHSKRTYDSFQDELEDYIKVQKARGLEPKTCFRKIGDSSVETHRNREMVDVRPRHRMLEQKLPCETFQTYSGPYSISQVVENQLPHCLPAHDSKQRLDSISYCQLTRDCFPEKPVPLSLNQQENNSGSYSVESEVYKHLSSENNTADHQAGHKQKHQKRKRHLEEGKERPEKEQSKHKRKKSYEDTDLDKDKSIRQRKREEDRVKVSSGKLKHRKKKKSHDVPSEKEERKHRKEKKKSVEERTEEEMLWDESILGF

SEQ ID NO: 10 - RNA, 4004 nt, Homo sapiens
  source 1..4004: mol_type = mRNA
gtccgcgcgcctcgctgcgatggcggcggcgccgagcacagtcaccccgctggcggcggagtcttcccctcaggaagctaccgtctctgccgcctcctcctcctcctacaccgcctgcgcggcagcggcggcggcggcggcggcggcggcagtaattgttcctgcctcctccgccacctccgcccctgcctgcccgcctgccggtggctgtggcgacggcggcggcggcggctttggcggctccactatggcggcggcggggagggggggcagtagttttaaagtagacacccgcccttgcctcagagaagacgccatttggaatgaacaggaaaaggctgaactttttacagataagttttgtcaagtatgtggagtgatgctacagtttgaatcacaaagaatttcacattatgagggtgaaaaacatgctcaaaatgttagtttttattttcaaatgcatggggaacaaaatgaagtgcctggtaagaaaatgaagatgcatgttgagaattttcaggtgcataggtatgaaggagtggacaaaaacaaattttgtgatctctgcaacatgatgtttagctctccacttattgctcagtctcactatgtgggaaaggtccatgctaaaaaactgaagcaattaatggaggaacatgatcaggcatctccatcaggatttcaaccagagatggcaggagtgcctcttactacttctgcagagtcaacattcctgaagccccttcctgtcaagcctcctacaggcaccttctcactactagtgtattttattcccctttcatattctctcttccacccacttcttttcctccactcactattatttttcagttctttctttacccagcctttctccttcatgtgaaaatgttcattcttctctcctccttgaagctctattctttgtcatatgtaactctttatgctctggttcttctctatttctgtttcttttttgttttctctatgggctctttttttattcattacctaaatgtaggtattcccatggtttcatccttgaccttactctacaaacacgatttctatttccctgatttcatttctcatctgtatgcttatgacctttctgtcaagtattagattcacatatccaaatggcagctactgcttcacctggatgcctcatagccatttcaaatccacctagtcattcaaagtagaaacccacagctcagatcttattagcacaggaatggctaccataacgatatggaactaaaaattttgctttcagcttttatcctgagagtggggctcactgaacctctctcctttctaaaatctgtggaaattacttttggtaactaaggcactgcagtgatctcagcatttagtatgagaacctatgtttgccatatttgtagtattgcttttacatctttagatatgttccggtcccacatgcaaggaagtgaacatcaaattaaagaatccattgttatcaatctagtgaagaattcaaggaagacacaagactcttaccaaaatgagtgtgcagattacatcaatgtgcagaaagccagaggactagaggccaagacttgtttcagaaagatggaagagagttctttggaaacccgtagatacagagaagtggtcgattccagacccagacatagaatgtttgaacaaagactcccatttgagactttccggacatacgcagcaccatacaatatttcacaagcaatggaaaagcagttacctcattcaaagaagacatatgactctttccaagatgaacttgaagattacatcaaagtacagaaagccagaggactagatccaaagacttgtttcagaaagatgagagagaactctgtggatactcatgggtacagagaaatggttgattctggacccagatcaagaatgtgtgagcaaagattttcacatgaggcttcccagacctaccaacgaccataccatatttcaccagtggaaagccagttacctcagtggctaccaacccattcaaagaggacatatgattctttccaagatgaacttgaagattacataaaagtgcagaaagccagaggactagagccaaaaacttgtttcagaaagataggagatagctctgtagaaacacacaggaacagagaaatggttgatgtcagacccagacatagaatgttggagcaaaagctcccatgtgagactttccagacctattcaggaccatatagtatttcacaagtagtggaaaaccagttacctcattgcttaccagctcatgatagcaaacagagactagattctattagctactgtcaactcaccagagactgtttcccagaaaaaccagtacccttgagccttaatcagcaagaaaataactctggctcatacagtgtagaatctgaagtttacaagcacctctcttcagaaaacaatactgctgaccatcaagcaggtcataaacggaaacatcagaagagaaaacgacacctagaagaaggcaaagaaaggccagagaaagagcagtccaagcataaaaggaaaaagagttatgaagatacagatttagacaaagacaagagcatcagacaaaggaaaagagaggaggatagagtcaaggtcagttcaggaaagcttaagcatcgaaaaaagaaaaaaagccatgatgtaccctccgagaaagaagaacgtaagcacaggaaagagaaaaagaaatctgttgaagaaaggacagaagaggaaatgctttgggatgagtctattcttggattttgaatgtttagttttgtttacccaaggttgaattgaaaaaaaaaaacagtcaatatggatttagaaaaaggaacacctgatgaagaaaaggagaggtagatacagtcagtgtcacttcaggacacttaggttttttttgtataaaaatttaaattgaattaaaagaaggaaaaaaaaagcccaaacttaacctctgagaaaagaacataagaactcaaggagaacataagagaaaaggaaacctgttacagaaaagacaagaatctgtgttttggaatgagtctattcttgggtattgaacttttagttttgtttgcccaaggattaattgaggaaatcagctaagaaaatggactttagacaaaagcaagaggatcagatgaagaaaaggagaggtagatacagtcagtgtcacttcaggaaagctatttaaaaaaacttgaaatttaattgaaagaagaaacaacaacaaaaaagcctaaacctagcctctgaacaacactaacatgagaacacaagaacttaagagaaaaagaaacctactcaagaaaagacagaagagacagtgatttgggatgagtctactctaggattttcaactttttagttttgttccttcaaagttgaaggaaaaaaagtttggttttataaaattcatgttattgtaatttttctaggtggatggctactttaatctctaaaaaagccaagtgaagtaaaagtattcagtatgccttttcctcaagttactttccttcattttcttaaaaaagaaaaaaaattattaaatgtttctcacatatctcacatataatgtaatttccctaaatgaagttgtctctacttctgctcatcaaattgctgtgatagtgaattatttattcatgggagataatttattttaaaggacagaattaccaagcgttacaaaatcagttctttccttggttttgtgttagtgttggtggtattttattgttgtttttctgtgtttatgtgtctcagctttctccaaggaatatgtatgaaataacttaaactgattttttctttgttaaatctaatttgcagtgtatttttgcattttctagttctgaaagtggaaaatgaaacagtctataataaacttagatgatatatagttttaaaacggtctcaaaaagtactgatataaggtcagtctatattctggaaatgtttatattaaagtgttttaatttcta

SEQ ID NO: 11 - protein, 175 aa, Homo sapiens
MPKRSCPFADVAPLQLKVRVSQRELSRGVCAERYSQEVFEKTKRLLFLGAQAYLDHVWDEGCAVVHLPESPKPGPTGAPRAARGQMLIGPDGRLIRSLGQASEADPSGVASIACSSCVRAVDGKAVCGQCERALCGQCVRTCWGCGSVACTLCGLVDCSDMYEKVLCTSCAMFET

SEQ ID NO: 12 - RNA, 752 nt, Homo sapiens
  source 1..752: mol_type = mRNA
gtcgttggtaaggggctggcggccggggagctgcgtagctcccggccccgcggccatgcccaagcggagctgccccttcgcggacgtggccccgctacagctcaaggtccgcgtgagccagagggagttgagccgcggcgtgtgcgccgagcgctactcgcaggaggtcttcgagaagaccaagcgactcctgttcctcggggcccaggcctacctggaccacgtgtgggatgaaggctgtgccgtcgttcacctgccagagtccccaaagcctggccctacaggggccccgagggctgcacgtgggcagatgctgattggaccagacggccgcctgatcaggagccttgggcaggcctccgaagctgacccatctggggtagcgtccattgcctgttcctcatgcgtgcgagccgtggatgggaaggcggtctgcggtcagtgtgagcgagccctgtgcgggcagtgtgtgcgcacctgctggggctgcggctccgtggcctgtaccctgtgtggcctcgtggactgcagtgacatgtacgagaaagtgctgtgcaccagctgtgccatgttcgagacctgaggctggctcaagccggctgccttcaccgggagccacgccgtgcatggcagccttccctggacgagcgctcggtgttcacactgaactgtggggtcgacgggaggggtgccttttacatgttctattttgtatcctaatgacagaatgaataaacctctttatatttgca

SEQ ID NO: 13 - protein, 496 aa, Homo sapiens
MDQDYERRLLRQIVIQNENTMPRVTEMRRTLTPASSPVSSPSKHGDRFIPSRAGANWSVNFHRINENEKSPSQNRKAKDATSDNGKDGLAYSALLKNELLGAGIEKVQDPQTEDRRLQPSTPEKKGLFTYSLSTKRSSPDDGNDVSPYSLSPVSNKSQKLLRSPRKPTRKISKIPFKVLDAPELQDDFYLNLVDWSSLNVLSVGLGTCVYLWSACTSQVTRLCDLSVEGDSVTSVGWSERGNLVAVGTHKGFVQIWDAAAGKKLSMLEGHTARVGALAWNAEQLSSGSRDRMILQRDIRTPPLQSERRLQGHRQEVCGLKWSTDHQLLASGGNDNKLLVWNHSSLSPVQQYTEHLAAVKAIAWSPHQHGLLASGGGTADRCIRFWNTLTGQPLQCIDTGSQVCNLAWSKHANELVSTHGYSQNQILVWKYPSLTQVAKLTGHSYRVLYLAMSPDGEAIVTGAGDETLRFWNVFSKTRSTKVKWESVSVLNLFTRIR

SEQ ID NO: 14 - RNA, 3170 nt, Homo sapiens
  source 1..3170: mol_type = mRNA
gctaaccttgccgcgggccgagccctgcctcgccatggaccaggactatgagcggcgcctgcttcgccagatcgtcatccagaatgagaacacgatgccacgcgtcacagagatgcggcggaccctgacgcctgccagctccccagtgtcctcgcccagcaagcacggagaccgcttcatcccctccagagccggagccaactggagcgtgaacttccacaggattaacgagaatgagaagtctcccagtcagaaccggaaagccaaggacgccacctcagacaacggcaaagacggcctggcctactctgccctgctcaagaatgagctgctgggtgccggcatcgagaaggtgcaggacccgcagactgaggaccgcaggctgcagccctccacgcctgagaagaagggtctgttcacggtgacgcggctctgtgacctctcagtggaaggggactcagtgacctccgtgggctggtctgagcgggggaacctggtggcggtgggcacacacaagggcttcgtgcagatctgggacgcagccgcagggaagaagctgtccatgttggagggccacacggcacgcgtcggggcgctggcctggaatgctgagcagctgtcgtccgggagccgcgaccgcatgatcctgcagagggacatccgcaccccgccactgcagtcggagcggcggctgcagggccaccggcaggaggtgtgcgggctcaagtggtccacagaccaccagctcctcgcctcggggggcaacgacaacaagctgctggtctggaatcactcgagcctgagccccgtgcagcagtacacggagcacctggcggccgtgaaggccatcgcctggtccccacatcagcacgggctgctggcctcggggggcggcacagctgaccgctgtatccgcttctggaacacgctgacaggacaaccactgcagtgtatcgacacgggctcccaagtgtgcaatctggcctggtccaagcacgccaacgagctggtgagcacgcacggctactcacagaaccagatccttgtctggaagtacccctccctgacccaggtggccaagctgaccgggcactcctaccgcgtgctgtacctggcaatgtcccctgatggggaggccatcgtcactggtgctggagacgagaccctgaggttctggaacgtctttagcaaaacccgttcgacaaaggagtctgtgtctgtgctcaacctcttcaccaggatccggtaaacctgccgggcaggaccgtgccacaccagctgtccagagtcggaggaccccagctcctcagcttgcatggactctgccttcccagcgcttgtcccccgaggaaggcggctgggcgggcggggagctgggcctggaggatcctggagtctcattaaatgcctgattgtgaaccatgtccaccagtatctggggtgggcacgtggtcggggaccctcagcagcaggggctctgtctcccttcccaaagggcgagaaccacattggacggtcccggctcagaccgtctgtactcagagcgacggatgccccctgggaccctcactgcctccgtctgttcatcacctgcccaccggagccgcatgctcttcctggaactgcccacgtctgcacagaacagaccaccagacgccagggctgattggtgggggcctgagaccccggttgcccattcatggctgcaccccaccatgtcaaacccaagaccagccccaaggccagaccaaggcatgtaggcctgggcaggtggctcggggccactggcggagccagcctgtggatccaagagacagtccccacctgggcttcacggcatccttgcagccacctctgctgtcactgctcgaagcagcagtctctctggaagcatctgtgtcatggccatcgcccggcggtcagtgggcttcagatgggcctgtgcatcctggccaagcgtcaccctcacactggaggaggatgtctgctctggacttatcaccccaggagaactgaacccggacctgctcactgccctggctggagaggagcacaacagatgccacgtcttcgtgcattcgccaacacgtgccctcacagggccagcgtcctccttccctgcgcaagacttgcgtcccccatgcctgctgggtggctgggtcctgtggaggccagcagcggtgtggcccccgcccccaggctgcctgtgtcttcacctgtcctgtccaccagcgccaacagccgtggggaagccaaggagacccaaggggtccaggaggtgggcgccctccatccttcgagaagcttcccaggctcctctgcttctctgtctcatgctcccaggctgcacagcaggcagggagggaggcaaggcaggggagtggggcctgagctgagcactgccccctcacccccccaccaccccttcccatttcatcggtggggacgtggagagggtggggcgggctggggttggagggtcccacccaccaccctgctgtgcttgggaacccccactccccactccccacatcccaacatcctggtgtctgtccccagtggggttggcgtgcatgtgtacatatgtatttgtgacttttctttggatttgttttgtgtttttgttgactagtcctggaaatgtttgaggctagacggggaggggccaggacccacccactgctcctgggggatgaggtcctggttttaaagccccgtcatttcaagcgggtcgatcttccacattcactggagagactctccccacctctgtctgggtggggcgcggacccctcactgtgcgcctgtgcagggggtgctggtgcacgtggcagtgtggatttccagtggtcacggtcttactgtttcaaggtttttaaataagaaaaccaaccctgccttcgcccatgcccgcccctgcccgcagttgccaaagagccgccttgtcgctgtgggcgtcagggcttggctggctcagtgcacaacccacagtggccttcagaggctcctcctgggactgggaaccgccgcagggccaggcggacggcgtgaggtttgtgttggggctggttctgcccatgctagggggtgggggagctcccaggacagaccagccttgtttctcatgtaatgcagtgacgctgtcattaaacacgtggattcatgtgtgg

SEQ ID NO: 15 - protein, 127 aa, Homo sapiens
MPKSKELVSSSSSGSDSDSEVDKKLKRKKQVAPEKPVKKQKTGETSRALSSSKQSSSSRDDNMFQIGKMRYVSVRDFKGKVLIDIREYWMDPEGEMKPGRKGISLNPEQWSQLKEQISDIDDAVRKL

SEQ ID NO: 16 - RNA, 3522 nt, Homo sapiens
  source 1..3522: mol_type = mRNA
gccccatcacgtgaccgcagccccagcgcggcggggccggcgtctcctggctgccgtcacttccggttctctgtcagtcgcgagcgaacgaccaagagggtgttcgactgctagagccgagcgaagcgatgcctaaatcaaaggaacttgtttcttcaagctcttctggcagtgattctgacagtgaggttgacaaaaagttaaagaggaaaaagcaagttgctccagaaaaacctgtaaagaaacaaaagacaggtgagacttcgagagccctgtcatcttctaaacagagcagcagcagcagagatgataacatgtttcagattgggaaaatgaggtacgttagtgttcgcgattttaaaggcaaagtgctaattgatattagagaatattggatggatcctgaaggtgaaatgaaaccaggaagaaaaggtatttctttaaatccagaacaatggagccagctgaaggaacagatttctgacattgatgatgcagtaagaaaactgtaaaattcgagccatataaataaaacctgtactgttctagttgttttaatctgtctttttacattggcttttgttttctaaatgttctccaagctattgtatgtttggattgcagaagaatttgtaagatgaatacttttttttaatgtgcattattaaaaatattgagtgaagctaattgtcaactttattaaggattactttgtctgcccaccacctagtgtaaaataaaatcaagtaatacaatcttaactgttgtggccttttttgatcataagagttggtactgtttaaggccaaaagtaacagtttttatagatcttttagtttcaactcagcttttacaataaaaaggatttgtattgcattgagtttataaacttttggtttgtgaacttcatatttgatcttttctcttccaatcaaatgtctaggcttgtttgacttccacccccaatggtttttcactctttttatttacttcattttcctttaataacttaatctcttcatgttcagtttttacttcactctttattcttttctttgattatggtatgcttatttggaaagtcagtgaaactgtcaaaatgttatctcaataagatacttatatgagaactacaatcaccgaatctactgtattcaatattagcagatctaatttgataaacaacatggcttgtgtgaaaactgagcaggtgtttgtttacccatagtgttctgtgtagttattgcttagtctgcagaaaataatgacttagatgagatgtctgacttgctttcacttattaaacatgttcaccatgggatgatgtctgtaacatcagatattgttcaactagactaggatttaataaaaattgtgaaagcttactggcctaacattttattttataatattgggtatgaattatatgtagccagagatgtcattaagctttactgttatagtaggtaatatggttagtttgtagggaaaagagcatatgagcacatgcttgtgtattttggcctttgccccagtagaacagaccaatggcattctagacttgatgatactaagttttagcagacactagtaagtggtttgtatttaaccatactgatgaagcagacagattgaggcacagattttagtggctttgtggcaataaatagggcatggtgtgccttaggaaaagaatgtttataaagggaattataactgaaattaaaggaggcggcagtgaagaggaaataattctcttctatctaaatgatatacatatgatattttgagatttttataacagcagtggaacacaattctaggtagagtagaaaaaggaaagttttaaagacatataaaagattcttgttgacaaattatttttggtagcaaatctcaaatggttacctgctattaaggtctgccatattagagttttgcactattttgctaccaagtttgattcatacatctaaaacattttgtagttacttgtcaaggacttaatttgaaaatcatttgccaggccacatagttatcaatttttttttctatcagctattctgttgtatttctaaaacattttttagatgactttttaaagtatatttagcagtaaccttatgaggttcaaattggtaaatctcttgtaatttagccttcatcgaataataggtaccagtgtattaaaaatgtgtattttttgcagccccttgaaccagagtaggttcagagaaactcccaaagtttgtactttagacacatcatgcttgattggtaacttccctccttttttggggaacatgtttgtgtcctattaacttaattggatagatttttaaatatttcttatttttggcacacggaaagggtagttcgagtacagaactttgatttttggtgtagatgcagagggaatgatgggtaaatttcctaggtttatgtgaatttagggggtgtatgcattttgaaacaatctactaacagatggtgctgaaatctattacctacatgttttctagttgttcagcattatgttaatgaagcctccatataaggagtgtttctctggcacagttggtaagttgactgctaacttcatttaaatgtgttactggatatgcagtatactgaaattattaatcagtttgtgtataggaaaagagaactgggttaaaagcaaattaacttgttctgaaaagaaagtatagattaattttgttttctgtttaaattttatctccttggtaaagatttttttttcctgggcagaaaacttggcatttttaggcgtagataccttaccttacaatgccaaaatgaatttaattccagtactcaggtttttccctttaacagactctatgtgtatcagggctttctaatgggtttttcctcttcgtttttaaaatgtgagtagcatttgaccaatttccagtgctcttagcattttacttaaagaacaaccactacaaaagaaaatctttgtaatttgattgtcttttgctttgcttcattaatgcctaagaacttaagaatactcctacctcattagctactcaagatgctgtgacgatcaaatctattctacataatgcgtttagaaacaaagacttgggtgaaaaatgaaataagtatattctgacttggctattgaggggaaaattcagtattaagtgttcctcacaggagatatgttagcagaatactataaaagtttgaaatttttaaaaagtaaaagtacttaaatttaggtatctctcctgaaattctttgcagttcattttttatggcagttaatccagtgaaacactcaaaagtttttttttttttaaaagtgtttttccagataaactgtagggtgaacattcacataatcacaaatatgtaattctgtaattgtggaatgcttgtatgctttgttttcgtacatcttccatggagatgtctgaatataatactccatctgtgaatattttaaatgttgaaataaaagtaagaaatgtgaaaaaaaaaaaaaaa

SEQ ID NO: 17 - protein, 112 aa, Human immunodeficiency virus 2
MTDPRERVPPGNSGEETIGEAFEWLDRTIEALNREAVNHLPRELIFQVWQRSWRYWHDDQGMSPSYTKYRYLCLMQKAVFIHFKRGCTCLGGGHGPGGWRSGPPPPPPPGLV

SEQ ID NO: 18 - protein, 112 aa, Human immunodeficiency virus 2
MTDPRETVPPGNSGEETIGEAFAWLNRTVEAINREAVNHLPRELIFQVWQRSWRYWHDEQGMSESYTKYRYLCIMQRAVYMHVRKGCTCLGRGHGPGGWRPGPPPPPPPGLV

SEQ ID NO: 19 - protein, 112 aa, Simian immunodeficiency virus
MSDPRERIPPGNSGEETIGEAFDWLDRTVEEINRAAVNHLPRELIFQVWRRSWEYWHDEMGMSVSYTKYRYLCLIQKAMFMHCKKGCRCLGGEHGAGGWRPGPPPPPPPGLA

SEQ ID NO: 20 - DNA, 21 bp, synthetic construct
  misc_feature 1..19: note = RNA
  misc_feature 20..21: note = DNA
  source 1..21: mol_type = other DNA
gtgccgagctaatacagtatt

SEQ ID NO: 21 - DNA, 21 bp, synthetic construct
  misc_feature 1..19: note = RNA
  misc_feature 20..21: note = DNA
  source 1..21: mol_type = other DNA
tactgtattagctcggcacgg

SEQ ID NO: 22 - DNA, 21 bp, synthetic construct
  misc_feature 1..19: note = RNA
  misc_feature 20..21: note = DNA
  source 1..21: mol_type = other DNA
caatgactcatgacgaagatt

SEQ ID NO: 23 - DNA, 21 bp, synthetic construct
  misc_feature 1..19: note = RNA
  misc_feature 20..21: note = DNA
  source 1..21: mol_type = other DNA
tcttcgtcatgagtcattgca

SEQ ID NO: 24 - DNA, 21 bp, synthetic construct
  misc_feature 1..19: note = RNA
  misc_feature 20..21: note = DNA
  source 1..21: mol_type = other DNA
gatgatgacttcataaagatt

SEQ ID NO: 25 - DNA, 21 bp, synthetic construct
  misc_feature 1..19: note = RNA
  misc_feature 20..21: note = DNA
  source 1..21: mol_type = other DNA
tctttatgaagtcatcatcct

SEQ ID NO: 26 - DNA, 21 bp, synthetic construct
  misc_feature 1..19: note = RNA
  misc_feature 20..21: note = DNA
  source 1..21: mol_type = other DNA
ggattaacgagaatgagaatt

SEQ ID NO: 27 - DNA, 21 bp, synthetic construct
  misc_feature 1..19: note = RNA
  misc_feature 20..21: note = DNA
  source 1..21: mol_type = other DNA
ttctcattctcgttaatcctg

SEQ ID NO: 28 - DNA, 21 bp, synthetic construct
  misc_feature 1..19: note = RNA
  misc_feature 20..21: note = DNA
  source 1..21: mol_type = other DNA
gcaaaacccgttcgacaaatt

SEQ ID NO: 29 - DNA, 21 bp, synthetic construct
  misc_feature 1..19: note = RNA
  misc_feature 20..21: note = DNA
  source 1..21: mol_type = other DNA
tttgtcgaacgggttttgcta

SEQ ID NO: 30 - DNA, 21 bp, synthetic construct
  misc_feature 1..19: note = RNA
  misc_feature 20..21: note = DNA
  source 1..21: mol_type = other DNA
gtatcgcatttctacagcatt

SEQ ID NO: 31 - DNA, 21 bp, synthetic construct
  misc_feature 1..19: note = RNA
  misc_feature 20..21: note = DNA
  source 1..21: mol_type = other DNA
tgctgtagaaatgcgatactt

SEQ ID NO: 32 - DNA, 21 bp, synthetic construct
  misc_feature 1..19: note = RNA
  misc_feature 20..21: note = DNA
  source 1..21: mol_type = other DNA
cgcaactctttacaccgtatt

SEQ ID NO: 33 - DNA, 21 bp, synthetic construct
  misc_feature 1..19: note = RNA
  misc_feature 20..21: note = DNA
  source 1..21: mol_type = other DNA
tacggtgtaaagagttgcgag

SEQ ID NO: 34 - DNA, 21 bp, synthetic construct
  misc_feature 1..19: note = RNA
  misc_feature 20..21: note = DNA
  source 1..21: mol_type = other DNA
gtgctaaacccaaagtatttt

SEQ ID NO: 35 - DNA, 21 bp, synthetic construct
  misc_feature 1..19: note = RNA
  misc_feature 20..21: note = DNA
  source 1..21: mol_type = other DNA
aatactttgggtttagcactg

SEQ ID NO: 36 - DNA, 21 bp, synthetic construct
  misc_feature 1..19: note = RNA
  misc_feature 20..21: note = DNA
  source 1..21: mol_type = other DNA
gcagtgacatgtacgagaatt

SEQ ID NO: 37 - DNA, 21 bp, synthetic construct
  misc_feature 1..19: note = RNA
  misc_feature 20..21: note = DNA
  source 1..21: mol_type = other DNA
ttctcgtacatgtcactgcag

SEQ ID NO: 38 - DNA, 21 bp, synthetic construct
  misc_feature 1..19: note = RNA
  misc_feature 20..21: note = DNA
  source 1..21: mol_type = other DNA
cctaatgacagaatgaatatt

SEQ ID NO: 39 - DNA, 21 bp, synthetic construct
  misc_feature 1..19: note = RNA
  misc_feature 20..21: note = DNA
  source 1..21: mol_type = other DNA
tattcattctgtcattaggat

SEQ ID NO: 40 - DNA, 21 bp, synthetic construct
  misc_feature 1..19: note = RNA
  misc_feature 20..21: note = DNA
  source 1..21: mol_type = other DNA
ggcagttaatggagaagtttt

SEQ ID NO: 41 - DNA, 21 bp, synthetic construct
  misc_feature 1..19: note = RNA
  misc_feature 20..21: note = DNA
  source 1..21: mol_type = other DNA
aacttctccattaactgccga

SEQ ID NO: 42 - DNA, 21 bp, synthetic construct
  misc_feature 1..19: note = RNA
  misc_feature 20..21: note = DNA
  source 1..21: mol_type = other DNA
gagccctcctgattttgaatt

SEQ ID NO: 43 - DNA, 21 bp, synthetic construct
  misc_feature 1..19: note = RNA
  misc_feature 20..21: note = DNA
  source 1..21: mol_type = other DNA
ttcaaaatcaggagggctctc

SEQ ID NO: 44 - DNA, 21 bp, synthetic construct
  misc_feature 1..19: note = RNA
  misc_feature 20..21: note = DNA
  source 1..21: mol_type = other DNA
agaaatccaggagaatgaatt

SEQ ID NO: 45 - DNA, 21 bp, synthetic construct
  misc_feature 1..19: note = RNA
  misc_feature 20..21: note = DNA
  source 1..21: mol_type = other DNA
ttcattctcctggatttcttc

SEQ ID NO: 46 - protein, 975 aa, Homo sapiens
MEDYEQELCGVEDDFHNQFAAELEVLAELEGASTPSPSGVPLFTAGRPPRTFEEALARGDAASSPAPAASVGSSQGGARKRQVDADLQPAGSLPHAPRIKRPRLQVVKRLNFRSEEMEEPPPPDSSPTDITPPPSPEDLAELWGHGVSEAAADVGLTRASPAARNPVLRRPPILEDYVHVTSTEGVRAYLVLRADPMAPGVQGSLLHVPWRGGGQLDLLGVSLASLKKQVDGERRERLLQEAQKLSDTLHSLRSGEEEAAQPLGAPEEEPTDGQDASSHCLWVDEFAPRHYTELLSDDFTNRCLLKWLKLWDLVVFGHERPSRKPRPSVEPARVSKEATAPGKWKSHEQVLEEMLEAGLDPSQRPKQKVALLCGPPGLGKTTLAHVIARHAGYSVVEMNASDDRSPEVFRTRIEAATQMESVLGAGGKPNCLVIDEIDGAPVAAINVLLSILNRKGPQEVGPQGPAVPSGGGRRRRAEGGLLMRPIICICNDQFAPSLRQLKQQAFLLHFPPTLPSRLVQRLQEVSLRQGMRADPGVLAALCEKTDNDIRACINTLQFLYSRGQRELSVRDVQATRVGLKDQRRGLFSVWQEVFQLPRAQRRRVGQDPALPADTLLLGDGDAGSLTSASQRFYRVLHAAASAGEHEKVVQGLFDNFLRLRLRDSSLGAVCVALDWLAFDDLLAGAAHHSQSFQLLRYPPFLPVAFHVLFASSHTPRITFPSSQQEAQNRMSQMRNLIQTLVSGIAPATRSRATPQALLLDALCLLLDILAPKLRPVSTQLYSTREKQQLASLVGTMLAYSLTYRQERTPDGQYIYRLEPNVEELCRFPELPARKPLTYQTKQLIAREIEVEKMRRAEASARVENSPQVDGSPPGLEGLLGGIGEKGVHRPAPRNHEQRLEHIMRRAAREEQPEKDFFGRVVVRSTAVPSAGDTAPEQDSVERRMGTAVGRSEVWFRFNEGVSNAVRRSLYIRDLL

SEQ ID NO: 47 - RNA, 3092 nt, Homo sapiens
  source 1..3092: mol_type = mRNA
gggcagtgcgcgacggcggcggcggcgcgggaggttcggagcgggagctcgggctcgcggacggtatggaggactacgagcaggagctgtgcggcgtcgaggatgatttccacaaccagttcgcggccgagctggaggtgctggcagagctggaaggggcgtcgactccgtcgccctccggggtccccctgttcaccgcgggccgacccccgcggacgttcgaggaggcccttgccagaggggacgcggcctccagtcccgccccagccgcatctgtgggcagcagccagggcggcgccaggaagaggcaggtggacgccgacctgcagccggccgggtccctgccccacgcccccaggatcaaacggcctaggctgcaggtggtcaagaggctgaacttcagatcggaggagatggaggagccgccccctcccgactcctcgccgacggacatcaccccgccgccgagccctgaggacctcgcagagctttggggccacggagtctcagaagctgctgccgacgtgggtctcacacgggcctcaccagctgcccgcaatcccgtcctgaggcggccccccatcttggaggactacgtccacgtgacatccacggagggcgtccgggcttatctggtgctgcgtgctgaccccatggccccgggggtgcagggctctctcctccacgtcccatggcgaggcggtggccagctggacctgctgggtgtgtccttagcctccctgaagaagcaggtcgacggcgagcggcgggagcggctgcttcaggaggcccagaagctttcagacaccctgcacagtctcaggtcgggggaggaggaggcagcccagcccttgggggcccctgaggaggagccgactgacggtcaagacgcctccagtcactgcctctgggtggatgagtttgcaccccgccactacacggagctgctcagtgatgacttcaccaaccgctgcctgctcaagtggctgaagttgtgggacctggtggtgtttggccacgagaggccttcccggaagcccaggcccagtgttgagccggcccgggtcagcaaggaggccacagccccaggcaagtggaagagccacgaacaggtgctggaggagatgctggaggctgggctggacccgagccagcgaccgaagcagaaggtggcactgctctgtgggcccccggggctggggaagaccaccctggcacacgtgattgcgcgtcacgcggggtactctgtggtggagatgaacgccagtgacgaccgtagcccggaggtcttccgcacacgcatcgaggcggccacccagatggagtcggtgctgggtgctggcgggaagcccaactgcctggtcatcgatgagatcgacggggcccccgtggccgccatcaacgtcctcctgagcatcctgaaccgcaaggggccacaggaggtggggccacagggcccggctgtgccttcgggaggcggccgacggcgccgggcagagggggggctcctcatgaggcccattatctgcatttgcaatgaccagttcgcaccgtccctgcggcagctgaagcagcaggccttcctgctccacttcccgccgactctgccctcgaggctggtgcagcggctccaggaggtctccctgcggcagggcatgagggccgacccaggggtgctggccgccctctgtgagaaaactgacaatgacatccgggcctgcatcaacaccctgcagttcctgtacagccggggccagcgggagctgagcgtgcgggacgtgcaggccacacgcgtgggcctcaaggaccagcgcagagggctcttctcggtgtggcaggaggtcttccagctgcctcgagcccagaggcgccgtgtgggccaggaccccgccctgcctgctgacacactcctgctgggtgacggggacgcgggctccctcacctccgcctcacagcgattctaccgtgtcctgcatgccgctgcctctgcgggcgagcacgagaaggtggtccagggcttgtttgacaacttcctgcgtctgcggctgcgagactccagcctgggtgctgtgtgtgtggccctcgactggctggccttcgatgacctgctggcgggggctgctcatcacagccagagcttccagctgctgcgctacccacccttcctgcccgtggccttccatgtgctgtttgcttccagccacacacccaggatcaccttccccagcagccagcaggaggcccagaaccggatgagccagatgaggaacctgatccagacgctggtgtccggcatcgcgccagccacgcgcagccgggccacgccccaggccctgctcctcgatgccctctgcctgctcctggacattcttgcacccaagctccgccccgtgagcacacagctgtacagcacccgtgaaaagcaacagctggccagcctggtgggcacgatgctcgcttacagcctgacctaccgccaggagcgcacgcccgatggccagtacatctacaggctggagccgaacgtggaggaactctgccgcttccctgagctgcctgcccgcaagcccctcacctaccagacgaagcagctcatcgcccgcgagatcgaggtggagaagatgcggcgggcggaggcttctgcccgggtagagaacagcccccaggtggatgggagccccccagggctcgagggtctgctggggggcattggggagaaaggggtgcaccgacctgccccacgcaaccatgagcagcggctggagcacatcatgaggcgagcggcccgggaggaacagcctgagaaggacttctttggacgtgtggtcgtcaggagcacagcagtcccgagtgcaggggacacggccccggagcaggactcagtggagcggcgcatgggcacagcggtgggcaggagcgaggtctggttccgcttcaacgagggtgtctccaacgccgtgcggcgcagcctgtacatcagggacttgctctagttctctgagccgcggacatgccctcgcattgcttcccgcagagtgcagagacaggaagctggagatgtctttataaagtcacacctttacagactgtaa

(19) United States
(12) Patent Application Publication
(10) Pub. No.: US 2026/0327620 A1
(43) Pub. Date: Oct. 22, 2026

(54) METHODS OF TREATING ALT-DEPENDENT CANCERS

(71) Applicant: CHILDREN'S MEDICAL RESEARCH INSTITUTE, Westmead, New South Wales (AU)

(72) Inventors: Roger Reddel, Westmead, New South Wales (AU); Karen Mackenzie, Westmead, New South Wales (AU); Yangxiu Wu, Westmead, New South Wales (AU)

(21) Appl. No.: 19/481,268

(22) PCT Filed: May 6, 2024
(86) PCT No.: PCT/AU2024/050443

§ 371 Date: Nov. 4, 2025

(30) Foreign Application Priority Data
May 5, 2023 (AU) 2023901353

(51) Int. Cl. C12N 15/113; A61K 31/713; A61P 35/00

(52) U.S. Cl. CPC C12N 15/1135; A61K 31/713; A61P 35/00; C12N 2310/11; C12N 2310/14

(57) ABSTRACT

The present disclosure generally relates to methods of treating cancer and more particularly ALT-dependent cancers.

### CROSS-REFERENCE TO RELATED APPLICATIONS

The present application claims priority from Australian Provisional Patent Application No. 2023901353 filed on 5 May 2023, the contents of which are incorporated herein by reference in their entirety.

### TECHNICAL FIELD

The present disclosure generally relates to methods of treating cancer and more particularly ALT-dependent cancers.

### BACKGROUND

Genomic integrity is maintained in normal cells in part by telomeres. The progressive shortening of telomeres through successive cell divisions limits the replicative potential of normal somatic cells. Replicative immortality is often achieved by cancer cells through overcoming telomere shortening by upregulation of a telomere length maintenance mechanism (TMM). In the majority of cancer cells, telomere length is maintained by telomerase. However, a significant population (<15%) of cancers employ telomerase-independent strategies, which are collectively referred to as Alternative Lengthening of Telomeres (ALT) (Bryan et al., 1995 and Bryan et al., 1997).

ALT-dependent cancer cells are characterized by elevated levels of DNA damage compared to mortal or telomerase-positive cells, which may indicate heightened telomeric replication stress in ALT-dependent cancer cells. This is attributed to cumulative inadequacies in telomere structural integrity. Frequent or persistent replication fork stalling causes nicks and breaks in the DNA, and it has been hypothesized that one ALT mechanism emanates from stalled replication forks that deteriorate to form double stranded breaks (DSBs), that then provide the substrate for the engagement of homology-directed repair pathways, culminating in break induced telomere synthesis. ALT telomeres therefore achieve a fine balance between telomere protection and telomere damage and repair activities, and disruption of this balance has the potential to dysregulate this ALT mechanism.

Many of the types of cancer where ALT-dependency is prevalent are biologically aggressive and difficult to treat (reviewed in Reddel R R. Curr Pharm Des. 2014; 20 (41): 6361-74). There are also currently no approved treatments that specifically target an ALT mechanism in cancer cells. Accordingly, there remains a clinically unmet need for new drug targets and improved treatments for ALT-dependent cancers.

### SUMMARY

The present disclosure is based on the identification of a number of additional protein molecules, including TERF2IP, SAMHD1, CCDC93, CHTF18, ETAA1, ZMAT1, SIVA1, FZR1 and SUB1, on which ALT-dependent cancer cells are dependent for survival. To this end, the present inventors have surprisingly shown that targeting these particular proteins can inhibit proliferation and/or induce cell death in ALT-dependent cell lines. On the basis of these findings, the inventors have developed new methods of inhibiting ALT cell viability and/or growth and methods of treating ALT-dependent cancer comprising inhibiting the expression and/or activity of one or more of TERF2IP, SAMHD1, CCDC93, CHTF18, ETAA1, ZMAT1, SIVA1, FZR1 and SUB1.

In a first aspect, the present disclosure provides a method of treating, preventing or ameliorating an ALT-dependent cancer in a subject, said method including the step of administering to the subject a therapeutically effective amount of an agent that reduces an expression and/or an activity of one or more of TERF2IP, SAMHD1, CCDC93, CHTF18, ETAA1, ZMAT1, SIVA1, FZR1 and SUB1 to thereby treat, prevent or ameliorate the ALT-dependent cancer in the subject.

In some examples, the present method further includes the earlier or initial step of identifying the ALT-dependent cancer in the subject.

In other examples, the present method further includes the step of administering a therapeutically effective amount of a further anti-cancer agent to the subject.

In a second aspect, the present disclosure provides a method of determining whether a cancer of a subject is responsive to or suitable for treatment with an agent that reduces an expression and/or an activity of one or more of TERF2IP, SAMHD1, CCDC93, CHTF18, ETAA1, ZMAT1, SIVA1, FZR1 and SUB1, said method including the step of determining an ALT status for the cancer of the subject, wherein a positive ALT status is indicative of the subject's cancer being responsive to or suitable for treatment with the agent.

In certain examples, the present method includes the further step of administering a therapeutically effective amount of the agent to the subject if the ALT status of the subject's cancer is positive.

According to the above aspects, the cancer or ALT-dependent cancer is suitably an osteosarcoma, a neuroblastoma, a gastric cancer, a liposarcoma, a glioblastoma, an astrocytoma or a bladder carcinoma.

In a third aspect, the present disclosure provides a method of reducing the viability and/or growth of an ALT-dependent cancer cell, said method including the step of contacting the ALT-dependent cancer cell with an effective amount of an agent that reduces an expression and/or an activity of one or more of TERF2IP, SAMHD1, CCDC93, CHTF18, ETAA1, ZMAT1, SIVA1, FZR1 and SUB1.

Suitably, the ALT-dependent cancer cell is an osteosarcoma cell, a neuroblastoma cell, a gastric cancer cell, a liposarcoma cell, a glioblastoma cell, an astrocytoma cell or a bladder carcinoma cell.

For the above aspects, the agent can be selected from the group consisting of a genetic inhibitor, a targeted nuclease, a small molecule, a peptide, a protein, an antibody or fragment thereof and any combination thereof. In particular examples, the genetic inhibitor is selected from the group consisting of siRNA, shRNA, antisense oligonucleotides, an aptamer, a targeted nuclease and any combination thereof. In other examples, the agent is or comprises HIV-2/SIV viral protein x (Vpx) or a derivative thereof.

In a fourth aspect, the present disclosure provides an agent that reduces an expression and/or an activity of one or more of TERF2IP, SAMHD1, CCDC93, CHTF18, ETAA1, ZMAT1, SIVA1, FZR1 and SUB1 for use in a method of treating, preventing or ameliorating an ALT-dependent cancer in a subject in need thereof.

In a fifth aspect, the present disclosure relates to the use of an agent that reduces an expression and/or an activity of one or more of TERF2IP, SAMHD1, CCDC93, CHTF18, ETAA1, ZMAT1, SIVA1, FZR1 and SUB1 in the manufacture of a medicament for use in treating, preventing or ameliorating an ALT-dependent cancer in a subject in need thereof.

In a sixth aspect, the present disclosure provides a method for identifying or producing an agent for use in the prevention, amelioration or treatment of an ALT-dependent cancer in a subject, said method including the steps of:
(a) contacting a cell that expresses one or more of TERF2IP, SAMHD1, CCDC93, CHTF18, ETAA1, ZMAT1, SIVA1, FZR1 and SUB1 with a candidate agent; and
(b) determining whether the candidate agent modulates an expression and/or an activity of one or more of TERF2IP, SAMHD1, CCDC93, CHTF18, ETAA1, ZMAT1, SIVA1, FZR1 and SUB1.

Suitably, the candidate agent, at least partly, reduces, eliminates, suppresses or inhibits the expression and/or the activity of one or more of TERF2IP, SAMHD1, CCDC93, CHTF18, ETAA1, ZMAT1, SIVA1, FZR1 and SUB1.

In some examples, the present method further includes the step of determining whether the candidate agent modulates the viability and/or growth of the cell. According to certain examples, the candidate agent, at least partly, reduces the viability and/or growth of the cell.

Suitably, the present method further includes the step of determining whether the candidate agent modulates a level of genomic stability at one or more telomeres in the cell and/or a level of ALT activity in the cell. In particular examples, the candidate agent, at least partly, increases the level of genomic stability at one or more telomeres in the cell and/or at least partly, reduces the level of ALT activity in the cell.

In some examples, the cell is or comprises an ALT-dependent cancer cell, such as an osteosarcoma cell, a neuroblastoma cell, a gastric cancer cell, a liposarcoma cell, a glioblastoma cell, an astrocytoma cell or a bladder carcinoma cell.

Suitably, the present method further includes one or more of the steps of:
selecting the candidate agent that modulates the expression and/or the activity of one or more of TERF2IP, SAMHD1, CCDC93, CHTF18, ETAA1, ZMAT1, SIVA1, FZR1 and SUB1;
isolating or purifying the candidate agent;
formulating the candidate agent into a pharmaceutical formulation; and
adding the candidate agent or the pharmaceutical formulation to packaging and/or a container.

In a seventh aspect, the present disclosure provides an agent produced according to the method of the sixth aspect, for use in treating, preventing or ameliorating an ALT-dependent cancer in a subject in need thereof.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1. Schematic of analysis of gene dependency data generated by the Wellcome Sanger Institute (WSI) and Broad Institute (BI) in relation to TMM classifications determined by Children's Medical Research Institute Cancer Research Unit (CMRI-CRU)

FIG. 2. Differential analysis of ALT v telomerase gene dependencies

FIG. 3. Heatmap of gene expression in cell lines

FIG. 4. Function of ALT dependent genes (all cell lines)

FIG. 5. WSI dependency data on known (A) and newly discovered (B) ALT targets identified from CMRI-CRU differential analysis

FIG. 6. Broad dependency data on known (A) and newly discovered (B) ALT targets identified from CMRI-CRU differential analysis

FIG. 7. Schematic of validation assay for ALT dependency and cell lines tested

FIG. 8. Validation assay of SAMHD1 dependency in ALT cell lines

FIG. 9. Validation assay of TERF2IP dependency in ALT cell lines

FIG. 10. Validation assay of FZR1 dependency in ALT cell lines

### KEY TO THE SEQUENCE LISTING

SEQ ID NO: 1 Amino acid sequence for a reference human TERF2IP protein
SEQ ID NO: 2 Nucleotide sequence for a reference human TERF2IP encoding mRNA
SEQ ID NO: 3 Amino acid sequence for a reference human SAMHD1 protein
SEQ ID NO: 4 Nucleotide sequence for a reference human SAMHD1 encoding mRNA
SEQ ID NO: 5 Amino acid sequence for a reference human CCDC93 protein
SEQ ID NO: 6 Nucleotide sequence for a reference human CCDC93 encoding mRNA
SEQ ID NO: 7 Amino acid sequence for a reference human ETAA1 protein
SEQ ID NO: 8 Nucleotide sequence for a reference human ETAA1 encoding mRNA
SEQ ID NO: 9 Amino acid sequence for a reference human ZMAT1 protein
SEQ ID NO: 10 Nucleotide sequence for a reference human ZMAT1 encoding mRNA
SEQ ID NO: 11 Amino acid sequence for a reference human SIVA1 protein
SEQ ID NO: 12 Nucleotide sequence for a reference human SIVA1 encoding mRNA
SEQ ID NO: 13 Amino acid sequence for a reference human FZR1 protein
SEQ ID NO: 14 Nucleotide sequence for a reference human FZR1 encoding mRNA
SEQ ID NO: 15 Amino acid sequence for a reference human SUB1 protein
SEQ ID NO: 16 Nucleotide sequence for a reference human SUB1 encoding mRNA
SEQ ID NO: 17 Amino acid sequence for a reference HIV-2 Vpx protein
SEQ ID NO: 18 Amino acid sequence for a reference HIV-2 Vpx protein
SEQ ID NO: 19 Amino acid sequence for a reference SIVsmm Vpx protein
SEQ ID NO: 20 Nucleotide sequence of sense strand of siCCDC93_1/a in Table 1
SEQ ID NO: 21 Nucleotide sequence of anti-sense strand of siCCDC93_1/a in Table 1
SEQ ID NO: 22 Nucleotide sequence of sense strand of siCCDC93_2/b in Table 1
SEQ ID NO: 23 Nucleotide sequence of anti-sense strand of siCCDC93_2/b in Table 1
SEQ ID NO: 24 Nucleotide sequence of sense strand of siCCDC93_3/c in Table 1
SEQ ID NO: 25 Nucleotide sequence of anti-sense strand of siCCDC93_3/c in Table 1
SEQ ID NO: 26 Nucleotide sequence of sense strand of siFZR1_1/a/(I) in Table 1
SEQ ID NO: 27 Nucleotide sequence of anti-sense strand of siFZR1_1/a/(I) in Table 1
SEQ ID NO: 28 Nucleotide sequence of sense strand of siFZR1_2/b/(II) in Table 1
SEQ ID NO: 29 Nucleotide sequence of anti-sense strand of siFZR1_2/b/(II) in Table 1
SEQ ID NO: 30 Nucleotide sequence of sense strand of siSAMHD1_1/a in Table 1
SEQ ID NO: 31 Nucleotide sequence of anti-sense strand of siSAMHD1_1/a in Table 1
SEQ ID NO: 32 Nucleotide sequence of sense strand of siSAMHD1_2/b/(I) in Table 1
SEQ ID NO: 33 Nucleotide sequence of anti-sense strand of siSAMHD1_2/b/(I) in Table 1
SEQ ID NO: 34 Nucleotide sequence of sense strand of siSAMHD1_4/d/(II) in Table 1
SEQ ID NO: 35 Nucleotide sequence of anti-sense strand of siSAMHD1_4/d/(II) in Table 1
SEQ ID NO: 36 Nucleotide sequence of sense strand of siSIVA1_1/a in Table 1
SEQ ID NO: 37 Nucleotide sequence of anti-sense strand of siSIVA1_1/a in Table 1
SEQ ID NO: 38 Nucleotide sequence of sense strand of siSIVA1_2/b in Table 1
SEQ ID NO: 39 Nucleotide sequence of anti-sense strand of siSIVA1_2/b in Table 1
SEQ ID NO: 40 Nucleotide sequence of sense strand of siTERF2IP_1/a in Table 1
SEQ ID NO: 41 Nucleotide sequence of anti-sense strand of siTERF2IP_1/a in Table 1
SEQ ID NO: 42 Nucleotide sequence of sense strand of siTERF2IP_3/c/(I) in Table 1
SEQ ID NO: 43 Nucleotide sequence of anti-sense strand of siTERF2IP_3/c/(I) in Table 1
SEQ ID NO: 44 Nucleotide sequence of sense strand of siTERF2IP_4/d (II) in Table 1
SEQ ID NO: 45 Nucleotide sequence of anti-sense strand of siTERF2IP_4/d (II) in Table 1
SEQ ID NO: 46 Amino acid sequence for a reference human CHTF18 protein
SEQ ID NO: 47 Nucleotide sequence for a reference human CHTF18 encoding mRNA

### DETAILED DESCRIPTION

### General Techniques and Definitions

Unless specifically defined otherwise, all technical and scientific terms used herein shall be taken to have the same meaning as commonly understood by one of ordinary skill in the art (e.g. in genomics, immunology, molecular biology, immunohistochemistry, biochemistry, oncology, and pharmacology).

The present disclosure is performed using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition, 1995), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment of the present disclosure may be applied mutatis mutandis to any other aspect or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

As used herein, the terms “approximately” and “about” refer to tolerances or variances associated with numerical values recited herein (e.g., +0.1%, 0.5%, 1.0%, 5.0% or 10%). The extent of such tolerances and variances are well understood by persons skilled in the art. Typically, such tolerances and variances do not compromise the structure, function and/or implementation of the compositions and methods described herein.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

By “consisting essentially of”, in the context of an amino acid sequence, is meant the recited amino acid sequence together with an additional one, two or three amino acids at the N- or C-terminus. Moreover, in the context of a nucleotide sequence, the term “consisting essentially of”, is meant the recited nucleotide sequence together with an additional one, two or three nucleic acid residues or bases at the 5′ end or 3′ end thereof.

All computer programs, algorithms, patent and scientific literature referred to herein is incorporated herein by reference.

For the present disclosure, the database accession number or unique identifier provided herein for a gene or protein, as well as the gene and/or protein sequence or sequences associated therewith, are incorporated by reference herein.

### Methods of Treating ALT-Dependent Cancers

The inventors have surprisingly shown for the first time that inhibition of TERF2IP, SAMHD1, CCDC93, CHTF18, ETAA1, ZMAT1, SIVA1, FZR1 or SUB1 can inhibit proliferation and promote cell death in ALT-dependent cancer cell lines. Therapeutically targeting these previously unknown mediators of an ALT mechanism therefore offers promise as potential therapies for ALT-dependent cancers.

Accordingly, in one form, the present disclosure provides a method of treating, preventing or ameliorating an ALT-dependent cancer in a subject, said method including the step of administering to the subject a therapeutically effective amount of an agent that reduces an expression and/or an activity of one or more of TERF2IP, SAMHD1, CCDC93, CHTF18, ETAA1, ZMAT1, SIVA1, FZR1 and SUB1 to thereby treat, prevent or ameliorate the ALT-dependent cancer in the subject.

In a related form, the present disclosure provides for the use of an agent that reduces an expression and/or an activity of one or more of TERF2IP, SAMHD1, CCDC93, CHTF18, ETAA1, ZMAT1, SIVA1, FZR1 and SUB1 in the manufacture of a medicament to treat, prevent or ameliorate an ALT-dependent cancer in a subject in need thereof.

In another form, the present disclosure relates to an agent that reduces an expression and/or an activity of one or more of TERF2IP, SAMHD1, CCDC93, CHTF18, ETAA1, ZMAT1, SIVA1, FZR1 and SUB1 for use in the treatment, prevention or amelioration of an ALT-dependent cancer in a subject in need thereof.

Methods of treating cancer may be prophylactic, preventative or therapeutic and suitable for treatment of cancer in mammals, particularly humans. As used herein, “treating”, “treat” or “treatment” refers to a therapeutic intervention, course of action or protocol that at least ameliorates a symptom of cancer after the cancer and/or its symptoms have at least started to develop. Desirable effects of treatment include decreasing the rate of disease progression, reducing size of the cancer, inhibiting tumour growth, inhibiting cancer progression or metastasis, ameliorating or palliating the disease state, and remission or improved prognosis. As used herein, “preventing”, “prevent” or “prevention” refers to a therapeutic intervention, course of action or protocol initiated prior to the onset of cancer and/or a symptom of cancer so as to prevent, inhibit or delay the development or progression of the cancer or the symptom. The term “ameliorate” as used herein, unless otherwise specified, means to eliminate, delay, or reduce the prevalence or severity of symptoms associated with a disease, disorder or condition.

The term “therapeutically effective amount” describes a quantity of a specified agent, such as an agent that inhibits or reduces an expression and/or an activity of one or more of TERF2IP, SAMHD1, CCDC93, CHTF18, ETAA1, ZMAT1, SIVA1, FZR1 and SUB1 or a composition described herein, sufficient to achieve a desired effect in a subject being treated with that agent or composition. For example, this can be the amount of the agent and optionally one or more further therapeutic agents (e.g., one or more further anti-cancer agents), necessary to reduce, alleviate and/or prevent an ALT-dependent cancer or a disease, disorder or condition associated therewith. Suitably, a “therapeutically effective amount” is sufficient to reduce or eliminate a symptom of an ALT-dependent cancer. More particularly, a “therapeutically effective amount” may be an amount sufficient to achieve a desired biological effect, for example an amount that is effective to decrease or prevent disease progression, such as cancer metastasis or recurrence, or overcome resistance to and/or enhance the anti-cancer activity of a further anti-cancer agent.

Ideally, a therapeutically effective amount of an agent is an amount sufficient to induce the desired result without causing a substantial cytotoxic effect in the subject. The therapeutically effective amount of an agent useful for reducing, alleviating and/or preventing the diseases, disorders and conditions described herein will be dependent on the subject being treated, the type and severity of any associated disease, disorder and/or condition (e.g., disease progression), and the manner of administration of the therapeutic composition.

As used herein, the term “subject” refers to any animal, for example, a mammalian animal, including, but not limited to humans, non-human primates, livestock (e.g. sheep, horses, cattle, pigs, donkeys), companion animals (e.g. pets such as dogs and cats), laboratory test animals (e.g. mice, rabbits, rats, guinea pigs), performance animals (e.g. racehorses, camels, greyhounds) or captive wild animals. In one embodiment, the subject is a human. For some examples, the subject is a female human, whilst in alternative examples, the subject is a male human. Typically, the terms “subject” and “patient” are used interchangeably, particularly in reference to a human subject. The subject may be receiving administration of (e.g., simultaneous, sequential or separate administration of) a further anti-cancer agent, such as those described herein (e.g., a chemotherapeutic agent). In alternative examples, however, the agent described herein is administered as the sole anti-cancer therapeutic modality or as a monotherapy. The subject may be a subject suffering from, suspected of suffering from, or predisposed to, cancer. The cancer may be any ALT-dependent cancer disclosed herein.

Suitably, the present methods further including the earlier or initial step of identifying whether the subject's cancer is an ALT-dependent cancer, such as by those methods described herein. In such instances, the present method may include the step of determining an ALT status and/or telomerase status of the subject's cancer.

Suitably, the present method further includes the step of administering a therapeutically effective amount of a further anti-cancer agent, such as those described herein, to the subject.

It is envisaged that the current methods can also improve the prognosis of the subject being treated. For example, administration of the agent that reduces an expression and/or an activity of one or more of TERF2IP, SAMHD1, CCDC93, CHTF18, ETAA1, ZMAT1, SIVA1, FZR1 and SUB1 to the subject with an ALT-dependent cancer may reduce the probability of a clinical worsening event (e.g., metastasis, cancer progression, cancer recurrence, death or a combination thereof) during the treatment period, and/or a modulation (i.e., an increase or decrease) from baseline in one or more biomarkers of disease progression. In some examples, the methods described herein provide a reduction of at least about 25%, at least about 50%, at least about 75% or at least about 80%, in probability of a clinical worsening event during the treatment period. In other examples, the current methods prevent a change of at least about 25%, at least about 50%, at least about 75% or at least about 80% of the concentration of one or more biomarkers of ALT-dependent cancer progression.

### Methods of Subject Selection

It is further envisaged that determining the ALT status and/or telomerase status of a cancer may be utilised in selecting subjects for treatment with an agent that reduces an expression and/or an activity of one or more of TERF2IP, SAMHD1, CCDC93, CHTF18, ETAA1, ZMAT1, SIVA1, FZR1 and SUB1. Similarly, determining levels of expression and/or activity of one or more of TERF2IP, SAMHD1, CCDC93, CHTF18, ETAA1, ZMAT1, SIVA1, FZR1 and SUB1 may be utilized in methods of identifying or determining an ALT status of a subject's cancer and hence whether the cancer in question may be sensitive to an agent designed for treating ALT-dependent cancers.

Accordingly, in one form, the present disclosure provides a method of determining whether a cancer of a subject is responsive to or suitable for treatment with an agent that reduces an expression and/or an activity of one or more of TERF2IP, SAMHD1, CCDC93, CHTF18, ETAA1, ZMAT1, SIVA1, FZR1 and SUB1, said method including the step of determining an ALT status for the cancer of the subject.

In another form, the present disclosure provides a method of determining whether a cancer of a subject is responsive to or suitable for treatment with an agent that inhibits an ALT mechanism, said method including the step of determining an expression and/or an activity of one or more of TERF2IP, SAMHD1, CCDC93, CHTF18, ETAA1, ZMAT1, SIVA1, FZR1 and SUB1, wherein the expression and/or activity thereof indicates or correlates with responsiveness of the subject's cancer to the agent. Suitably, an elevated or increased level of expression or activity, such as an interaction with telomere DNA, of one or more of TERF2IP, SAMHD1, CCDC93, CHTF18, ETAA1, ZMAT1, SIVA1, FZR1 and SUB1 indicates or correlates with increased responsiveness of the subject's cancer to the agent. Alternatively, a reduced or decreased level of expression and/or activity, such as an interaction with telomere DNA, of one or more of TERF2IP, SAMHD1, CCDC93, CHTF18, ETAA1, ZMAT1, SIVA1, FZR1 and SUB1 indicates or correlates with reduced or no responsiveness of the subject's cancer to the agent.

The terms “determining”, “measuring”, “evaluating”, “assessing” and “assaying” are used interchangeably herein and may include any form of measurement known in the art.

Accordingly, determining, assessing, evaluating or assaying an ALT status of a subject's cancer, may be performed by any technique known in the art, such as those methods described herein. In this regard, it is envisaged that determining an ALT status of the subject's cancer may include determining an ALT status and/or a telomerase status thereof. Suitable assays include, but are not limited to, the measurement of C-circles (e.g., as disclosed in Henson et al., 2009 and WO2011035375), quantitative PCR of telomeric DNA and C-circles (Lau et al., 2012), the absence of telomerase activity, the presence of very long and heterogeneous telomeres (Bryan et al., 1995), the presence of ALT-associated PML bodies (APBs) (Yeager et al. Cancer Res. 1999; 59 (17): 4175-9), elevated telomeric sister chromatid exchange (T-SCE) (for example, which is elevated relative to a non-ALT cell, such as a telomerase positive cell or a mortal cell) and detection of single-stranded telomeric DNA by the ALT-FISH assay (Frank et al. Nucleic Acids Res. 2022; 50 (11): e61).

Any of the methods disclosed herein for identifying an ALT-dependent cancer cell may be used to determine whether a subject is suffering from an ALT-dependent cancer. In particular examples, diagnosing an ALT-dependent cancer in a subject includes a C-circle assay and/or an ALT-FISH assay. These methods may be performed on the subject or on a sample, such as biopsy sample or biological sample, taken from a subject. Accordingly, the present method may include the initial or earlier step of obtaining a biological sample, such as a biopsy sample comprising one or more cells or tissues of the subject's cancer, from the subject.

As such, in another form, the present disclosure provides a method of determining whether a cancer of a subject is responsive to or suitable for treatment with an agent that reduces an expression and/or an activity of one or more of TERF2IP, SAMHD1, CCDC93, CHTF18, ETAA1, ZMAT1, SIVA1, FZR1 and SUB1, said method including the step of determining an ALT status and/or a telomerase status for the cancer of the subject.

According to certain examples, the current methods may further include treating the subject's cancer, such as based on the determination of the ALT status and/or the telomerase status thereof. As such, the present methods may include the step of administering a therapeutically effective amount of a therapeutic agent, such as the agent that reduces an expression and/or an activity of one or more of TERF2IP, SAMHD1, CCDC93, CHTF18, ETAA1, ZMAT1, SIVA1, FZR1 and SUB1 and/or a further anti-cancer agent.

In a broad form, a positive ALT status (irrespective of the telomerase status of the subject's cancer) is indicative of the subject's cancer being responsive to or suitable for treatment with the agent. More particularly, the present method includes the further step of administering a therapeutically effective amount of the agent to the subject if the subject's cancer is identified as having a positive ALT status (i.e., is identified as being an ALT-dependent cancer).

Suitably, a positive ALT status and/or a negative telomerase status is indicative of the subject's cancer being responsive to or suitable for treatment with the agent. More particularly, the present method includes the further step of administering a therapeutically effective amount of the agent to the subject if the subject's cancer is identified as having a positive ALT status and/or a negative telomerase status (i.e., is identified as being an ALT-dependent cancer).

Similarly, a positive ALT status and a positive telomerase status can be indicative of the subject's cancer being responsive to or suitable for treatment with the agent. More particularly, the present method includes the further step of administering a therapeutically effective amount of the agent to the subject if the subject's cancer is identified as having a positive ALT status and a positive telomerase status (i.e., is identified as being an ALT-dependent cancer).

Conversely, a negative ALT status and/or a positive telomerase status can be indicative of the subject's cancer being unresponsive to or not suitable for treatment with the agent. In such instances, the agent is suitably not administered to the subject if the subject's cancer is identified as having a negative ALT status and/or a positive telomerase status (i.e., is identified as not being an ALT-dependent cancer). In such instances, an alternative anti-cancer treatment, such as those provided herein may be administered to the subject.

In another form, the present disclosure provides a method of determining whether a subject is responding to treatment with an agent that reduces an expression and/or an activity of one or more of TERF2IP, SAMHD1, CCDC93, CHTF18, ETAA1, ZMAT1, SIVA1, FZR1 and SUB1, including the step of determining the presence and/or a level of: (a) genomic instability at one or more telomeres; and/or (b) ALT activity; in one or more cells or tissues taken from or obtained from the subject, and more particularly the subject's cancer. The presence and/or level of genomic stability and/or ALT activity may be determined using any suitable method known in the art, or using any of the methods disclosed herein for identifying an ALT-dependent cell.

The methods disclosed herein may comprise assaying a sample, such as a biological sample (e.g., a biopsy sample) comprising one or more cells or tissues of the subject's cancer, that may be obtained from the subject before treatment with the agent to determine the presence and/or level of genomic instability at one or more telomeres and/or the presence and/or level of ALT activity in a cell taken from the subject. This may be compared to a sample taken from the subject after treatment with the agent. Thus, an increase in genomic instability or a decrease in the level of ALT activity after treatment with the agent relative to the amount/presence of genomic instability or level of ALT activity before treatment with the agent is indicative that the subject is responding to treatment with the agent. Conversely, a lack of increase, no change or a decrease in genomic instability or a lack of decrease, no change or an increase in the level of ALT activity after treatment with the agent relative to the amount/presence of genomic instability or level of ALT activity before treatment with the agent is indicative that the subject is not responding to treatment with the agent.

It is further envisaged that assays which are used to determine whether a cell is an ALT-dependent cell, such as an ALT-dependent cancer cell or a cancer is an ALT-dependent cancer, may also be used to determine whether a subject is responding to treatment with the agent (e.g., determine the presence and/or a level of genomic instability at one or more telomeres; and/or ALT activity of a subject's cancer).

For example, any of the methods disclosed in Henson et al., 2009 and/or WO/2011/035375 can be used to determine whether a subject is responding to the treatment methods of present disclosure. Thus, for example, the methods disclosed herein may comprise assaying a sample obtained from a subject treated with the agent for the presence and/or amount of partially double-stranded telomeric circles, wherein the partially double-stranded telomeric circles are detected following rolling circle amplification using the partially double-stranded circular telomeric DNA as template, wherein the presence and/or amount of said circles in indicative of whether the subject is responding to treatment with the agent. The methods may be performed with or without an exogenous primer. For example, the methods may be performed without an exogenous primer. A sample, such as a biological sample (e.g., a biopsy sample of the subject's cancer) may be obtained from the subject prior to beginning treatment with the agent and after treatment with the agent. The presence and/or amount of C-circles in the samples may be compared to determine whether the subject is responding to treatment with the agent. For example, an absence or decrease in the presence, level and/or amount of C-circles in a subject's sample is indicative that the subject is responding to treatment with the agent.

Inhibiting ALT-Dependent Cancer Cell Viability and/or Growth

The present disclosure also provides methods of inhibiting ALT-dependent cancer cell viability and/or growth. In this regard, the present inventors have surprisingly shown that inhibiting one or more of TERF2IP, SAMHD1, CCDC93, CHTF18, ETAA1, ZMAT1, SIVA1, FZR1 and SUB1 is selectively toxic to ALT-dependent cancer cells.

As such, in one form, the present disclosure provides a method of inhibiting or reducing the viability and/or growth of an ALT-dependent cancer cell, said method including the step of contacting the ALT-dependent cancer cell with an effective amount of an agent that reduces an expression and/or an activity of one or more of TERF2IP, SAMHD1, CCDC93, CHTF18, ETAA1, ZMAT1, SIVA1, FZR1 and SUB1.

As used herein, the term “effective amount” and the like refers to an amount of the agent that is sufficient to induce a desired physiological outcome (e.g., at least partly reducing the viability and/or growth of the ALT-dependent cancer cell). An effective amount can be administered in one or more administrations, applications or dosages.

The term “reducing the viability and/or growth” is used to mean reducing the ability of an ALT-dependent cancer cell to survive and/or proliferate. Cell viability and/or growth may be inhibited in any measurable amount. Inhibition of cell viability and/or growth may be complete or may be partial. Thus, the methods disclosed herein may comprise at least partial inhibition of ALT-dependent cancer cell viability and/or growth.

Suitably, the viability and/or growth of the ALT-dependent cancer cell in the presence of the agent is less than about 99%, 98%, 97%, 96%, 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the viability and/or growth of a control or reference ALT-dependent cancer cell in the absence of the agent.

Viability of the ALT-dependent cancer cells may be assessed by any suitable means or method known in the art. Typically, cell viability is quantified as the percentage of living cells present (i.e., the number of live cells divided by the total number of cells). As cells are either live or dead, cell viability may be determined by quantifying either dead cells or live cells. One suitable method is trypan blue exclusion. Other assays for determining cell viability may include, without limitation, propidium iodide staining, TUNEL, Resazurin, methyl violet, lactate dehydrogenase, fluorescein diacetate hydrolysis, MTT, caspase, and ATP assays.

Growth or proliferation of the ALT-dependent cancer cells may also be measured by methods known in the art. Suitable methods include measuring the size of the cell population (e.g., by counting cells using a marker specific for the cell population, such as a tumour specific marker) or by performing cell cycle analysis using 5-bromo-2′-deoxyuridine (BrdU) which becomes incorporated into newly made DNA and propidium iodide (PI) and analysing by flow cytometry in combination with a cell population specific marker. Other suitable methods for measuring proliferation include proliferations assays, such as MTT assays, MTS assays, intracellular ATP assays, measuring cellular LDH activity, 3H-thymidine incorporation and the Cell Titer Glo Assay.

It is envisaged that the method of the present aspect may be performed in relation to cells in vitro, in vivo and/or ex vivo. In some examples, the method is performed in vitro, such as with ALT-dependent cancer cells isolated from a subject, patient-derived cells (immortalized or non-immortalized), a patient-derived tumour organoid or a patient-derived xenograft model. Accordingly, in some examples, the present method includes the earlier step of isolating the ALT-dependent cancer cells from the subject. In other examples, the present method is performed in vivo in a subject.

For the purposes of the present disclosure, by “isolated” is meant material that has been removed from its natural state or otherwise been subjected to human manipulation. Isolated material (e.g., ALT-dependent cancer cells) may be substantially or essentially free from components that normally accompany it in its natural state, or may be manipulated so as to be in an artificial state together with components that normally accompany it in its natural state.

The ALT-dependent cancer cell may be derived from any vertebrate, such as a mammal, and in particular, a human. Additionally, the ALT-dependent cancer cell may be derived from any cancer, such as those described herein. In particular examples, the ALT-dependent cancer cell is an osteosarcoma cell, a neuroblastoma cell, a gastric cancer cell, a liposarcoma cell, a glioblastoma cell, an astrocytoma cell or a bladder carcinoma cell. More particularly, the ALT-dependent cancer cell can be an osteosarcoma cell, a neuroblastoma cell or a gastric cancer cell.

The present method may include the initial or earlier step of determining an ALT status and/or a telomerase status of a cancer cell to identify an ALT-dependent cancer cell. Such determining, assessing, evaluating or assaying an ALT status and/or a telomerase status of a cancer cell, may be performed by any technique known in the art, such as those methods described herein.

According to certain examples, the present method further includes the step of contacting the ALT-dependent cancer cell with an effective amount of a further anti-cancer agent, such as those described herein.

With respect to the methods described herein, the agent may be any, such as those described herein, that reduces, inhibits or disrupts an expression and/or an activity of one or more of TERF2IP, SAMHD1, CCDC93, CHTF18, ETAA1, ZMAT1, SIVA1, FZR1 and SUB1. In particular examples, the agent reduces, inhibits or disrupts an expression and/or an activity of TERF2IP. In particular examples, the agent reduces, inhibits or disrupts an expression and/or an activity of SAMHD1. In particular examples, the agent reduces, inhibits or disrupts an expression and/or an activity of CCDC93. In particular examples, the agent reduces, inhibits or disrupts an expression and/or an activity of CHTF18. In particular examples, the agent reduces, inhibits or disrupts an expression and/or an activity of ETAA1. In particular examples, the agent reduces, inhibits or disrupts an expression and/or an activity of ZMAT1. In particular examples, the agent reduces, inhibits or disrupts an expression and/or an activity of SIVA1. In particular examples, the agent reduces, inhibits or disrupts an expression and/or an activity of FZR1. In particular examples, the agent reduces, inhibits or disrupts an expression and/or an activity of SUB1.

### ALT-Dependent Cancer

In order to achieve unlimited proliferation, cancer cells must maintain their telomeres. The majority of cancers (85-90%) achieve this by reactivating telomerase (telomerase-positive cells). Telomerase is a reverse transcriptase enzyme involved in synthesizing telomeric DNA from an RNA template. Some of the remaining 10-15% of tumour cells may stabilize their chromosome ends by alternative mechanisms to avert cessation of growth. These telomerase independent strategies are collectively known as Alternative Lengthening of Telomeres (ALT). Thus, an ALT-dependent cell or cancer as defined herein may be a cell or cancer exhibiting an active ALT mechanism.

Although the initial discovery of ALT-dependent cancers and cancer cells depended on being able to demonstrate that telomere length maintenance can occur in the absence of any detectable telomerase activity, more recently it has been shown that ALT and telomerase can co-exist in ALT-dependent cancers and cancer cells (i.e., some cancers and cancer cells that utilize an ALT mechanism also have telomerase activity or are telomerase positive). Accordingly, ALT-dependent cancers and cancer cells provided herein may or may not have any detectable telomerase activity. It is therefore envisaged that the presence of telomerase activity need not necessarily preclude a cancer or cancer cell from being ALT-dependent, whilst the absence of telomerase activity may be suggestive or indicative that a cancer or cancer cell could be ALT-dependent.

In view of the above and according to some examples, an ALT-dependent cancer or cancer cell has a positive telomerase status. In alternative examples, an ALT-dependent cancer or cancer cell does not have a positive telomerase status (i.e., are telomerase negative or have a negative telomerase status).

An ALT mechanism may be any mechanism of telomere stabilization that does not rely on telomerase. An ALT mechanism is not necessarily limited to any one specific mechanism by which ALT may operate in a cell to maintain telomeres. Thus, the term “ALT mechanism” does not necessarily refer to only one specific biochemical mechanism or pathway by which an ALT mechanism operates. There may be more than one specific pathway by which ALT operates.

ALT-dependent cells may be characterized by elevated levels of DNA damage compared to mortal or telomerase-positive cells, indicative of heightened telomeric replication stress in ALT-dependent cells. This heightened telomeric replication stress is attributed to cumulative inadequacies in telomere structural integrity. Frequent or persistent replication fork stalling causes nicks and breaks in the DNA, and it has been hypothesized that one particular ALT mechanism emanates from stalled replication forks that deteriorate to form double stranded breaks (DSBs), which then provide the substrate for the engagement of homology-directed repair pathways, culminating in break-induced telomere synthesis. These ALT-dependent cells therefore achieve a fine balance between telomere protection and repair activities and telomere damage, and disruption of this balance can be applied as a means of dysregulating this ALT mechanism.

The methods of the present disclosure may further include the earlier or initial step of identifying the presence of the ALT-dependent cancer in the subject. Similarly, the methods described herein may include the earlier or initial step of identifying a cancer cell as an ALT-dependent cancer cell. Accordingly, the methods of this disclosure may include the initial step of determining an ALT status and/or a telomerase status of a cancer cell or the subject's cancer, such as from a sample (e.g., a biopsy sample or a biological sample) obtained from the subject or the subject's cancer. Such a determining step may be performed by any means or method of testing known in the art.

Further to the above, it is contemplated that identifying an ALT-dependent cancer cell, identifying an ALT-dependent cancer or determining whether a subject is suffering from ALT-dependent cancer may comprise determining a telomerase status of said cancer or cancer cell (e.g., determining whether a cancer cell or a cell derived from the cancer is a telomerase positive cell or a telomerase negative cell by any of the methods disclosed herein, wherein the cell is identified as: (a) being or not being an ALT-dependent cell if that cell is determined to be a telomerase positive cell; or (b) being an ALT-dependent cell if that cell is determined to be a telomerase negative cell).

In order to assess the telomerase status of a cancer or cancer cell, the expression and/or activity of telomerase can be determined by any means known in the art. For example, telomerase expression levels can be determined by quantifying the level of production of telomerase mRNA by any suitable method of mRNA detection (for example, but without limitation: quantitative PCR, real time qPCR; next generation sequencing (NGS) methods; nanopore sequencing methods; northern blotting; and others). Alternatively or in addition, telomerase expression levels can be determined by quantifying the level of production of telomerase protein by any suitable protein detection methods. Telomerase protein levels can be detected, for example but without limitation, by western blotting; antibody detection methods (e.g., ELISA; or detection of a label such as a fluorescent label conjugated to an antibody capable of binding specifically to telomerase); and other methods. Alternatively or in addition, telomerase expression and/or activity levels can be determined through performance of a telomerase functional assay, wherein the level of telomerase activity is indicative of the level of expression and/or activity of telomerase in a cell. Telomerase activity may be detected by the Telomerase Repeat Amplification Protocol (TRAP), quantitative TRAP (qTRAP), or by a direct telomerase activity assay, such as that described in Cohen and Reddel, 2008.

ALT-dependent cancers or cancer cells may also be identified by detection of one or more phenotypic traits of ALT telomere repair or ALT telomeric replication stress, including any one or more of: replication fork stalling above a level that is typical of non-ALT cells (e.g., above a level that is typical of telomerase positive cells or mortal cells); DSB occurrence above a level that is typical of non-ALT cells (e.g., above a level that is typical of telomerase positive cells or mortal cells); and others. It will be appreciated that levels of telomeric replication fork stalling and/or DSBs that are typical of ALT-dependent cells can be established through identification and/or measurement of these traits in a sample of ALT-dependent cells and in a sample of non-ALT cells (e.g., telomerase positive cells or mortal cells). Suitable threshold levels can then be determined according to the particular methodology used to identify and/or measure these traits, such that a given cell can then be identified as an ALT-dependent cell or a non-ALT cell using the same or similar methodology. The precise thresholds can vary depending on the samples used to establish those threshold levels and according to the particular analytical methodology used in each instance.

Telomere length and other phenotypic traits, such as the presence of ALT-associated promyelocytic leukemia nuclear bodies (APBs), may also be utilised to determine an ALT status of a cancer or cancer cell. ALT involves recombination-dependent DNA replication (Dunham et al., 2000) and ALT may generate sudden, large increases in telomere length (Murnane et al., 1994), consistent with either a long, linear telomeric template or a rolling mechanism, such as rolling circle amplification (RCA). Cells with ALT activity also undergo rapid decreases in individual telomere lengths (Jiang et al., 2005 and Perrem et al., 2001) leading to a highly heterogeneous telomere length distribution. ALT cells often contain telomeric chromatin within promyelocytic leukemia (PML) nuclear bodies (ALT-associated promyelocytic leukemia nuclear bodies; APBs) (Yeager et al., 1999).

Thus, an ALT-dependent cancer or cancer cell as defined herein may comprise any one or more of the above phenotypic traits. For example, an ALT-dependent cell may exhibit recombination-dependent DNA replication, and/or may exhibit sudden, large increases in telomere length (e.g., compared to a non-ALT cell such as a telomerase positive cell or a mortal cell), and/or may exhibit heterogeneous telomere length distribution (e.g., compared to a non-ALT cell such as a telomerase positive cell or a mortal cell), and/or may comprise APBs (e.g., a level of APBs that is greater than in a non-ALT cell, such as a telomerase positive cell or a mortal cell). Alternatively, an ALT-dependent cell may be identified by the maintenance of telomere length over one or more cell divisions, in the absence of telomerase activity and/or expression. It will be appreciated that telomeres are repetitive DNA sequences present at or near the termini of linear chromosomes. Telomeres in humans typically comprise multiple repeats of the nucleotide sequence 5′-TTAGGG-3′. Thus, the identification of telomere length may comprise determining the number of repeats of this nucleotide sequence.

ALT-dependent cancers and cancer cells may also be identified by the presence of biomarkers. For example, the C-circle biomarker is an ALT specific molecule which can be detected using the C-circle assay (Henson et al., 2009 and WO/2011/035375). Thus, the methods disclosed herein may comprise identifying a cell as an ALT-dependent cell by determining the presence and/or amount of partially double-stranded telomeric DNA circles in a cell, wherein the presence and/or amount of partially double-stranded telomeric DNA circles identifies that cell as an ALT-dependent cell.

Significant differences in telomere variant repeat content have also been found in tumours that use an ALT mechanism and those that do not (Lee et al., 2018). Thus, any method known in the art to determine telomere variant repeat content may be used in conjunction with the present disclosure. For example, whole genome sequencing may be used to determine telomere variant repeat content.

Any of the methods disclosed herein may comprise a step of establishing a reference level of any one or more ALT-dependent cell characteristics and/or non-ALT cell characteristics. Alternatively, any of the methods disclosed herein may comprise a step of comparing a measurement of an ALT-dependent cell characteristic to a predetermined reference level.

As generally used herein, the terms “cancer”, “tumour”, “malignant” and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression.

Cancers may include any aggressive or potentially aggressive cancers, tumours or other malignancies such as listed in the NCI Cancer Index at http://www.cancer.gov/cancertopics/alphalist, including all major cancer forms such as sarcomas, carcinomas, lymphomas, leukaemias and blastomas, although without limitation thereto. These may include breast cancer, lung cancer inclusive of lung adenocarcinoma, cancers of the reproductive system inclusive of ovarian cancer, cervical cancer, uterine cancer and prostate cancer, cancers of the brain and nervous system, head and neck cancers, gastrointestinal cancers inclusive of colon cancer, colorectal cancer and gastric cancer, liver cancer, kidney cancer, skin cancers such as melanoma and skin carcinomas, blood cell cancers inclusive of lymphoid cancers and myelomonocytic cancers, cancers of the endocrine system such as pancreatic cancer and pituitary cancers, musculoskeletal cancers inclusive of bone and soft tissue cancers, although without limitation thereto.

The presence of an ALT mechanism has been identified in carcinomas arising from tissue including tissue derived from the bladder, cervix, endometrium, stomach, esophagus, gallbladder, kidney, liver, lung, brain, bone and connective tissue. Evidence for an ALT mechanism has also been found in neuroblastomas, medulloblastomas, astrocytomas, oligodendrogliomas, meningiomas, schwannomas and pediatric glioblastomas. In certain examples, the ALT-dependent cancer provided herein may be any one of bladder cancer, cervical cancer, endometrial cancer (e.g., endometrial carcinoma), ovarian cancer, breast cancer, oesophageal cancer, gallbladder cancer, kidney cancer, liver cancer, lung cancer, brain cancer, nervous tissue cancer (e.g., neurofibroma, ganglioneuroblastoma), bone cancer (e.g., osteosarcoma, chondrosarcoma or chordoma), pancreatic neuroendocrine cancer, adrenal gland cancer (e.g., phaeochromocytoma), skin cancer (e.g., melanoma), epithelial cell cancer (e.g., mesothelioma), gastric cancer, blood cell cancer (e.g., leukemia) or connective tissue cancer (e.g., a soft tissue sarcoma). The ALT-dependent cancer may be, for example, a sarcoma, a blastoma, a carcinoma, a mesothelioma or an astrocytoma. The sarcoma may be osteosarcoma, malignant fibrous histiocytoma, liposarcoma, synovial sarcoma, fibrosarcoma, chondrosarcoma, rhabdomyosarcoma or leiomyosarcoma. The blastoma may be neuroblastoma. The carcinoma may be a non-small cell lung carcinoma such as lung adenocarcinoma, a breast carcinoma or an endometrial carcinoma. The mesothelioma may be peritoneal mesothelioma. The astrocytoma may be low-grade astrocytoma, anaplastic astrocytoma, or glioblastoma. The ALT-dependent cancer may be a medulloblastoma, oligodendroglioma, meningioma, schwannoma and/or pediatric glioblastoma. In certain examples, the ALT-dependent cancer cell is an osteosarcoma cell, a neuroblastoma cell, a gastric cancer cell, a liposarcoma cell, a glioblastoma cell, an astrocytoma cell or a bladder carcinoma cell. In other examples, the ALT-dependent cancer is an osteosarcoma, a neuroblastoma, a gastric cancer, a liposarcoma, a glioblastoma, an astrocytoma or a bladder carcinoma. More particularly, the ALT-dependent cancer cell or the ALT-dependent cancer is a gastric carcinoma, a neuroblastoma or an osteosarcoma. The cancer may be a primary cancer or a metastatic cancer. The metastatic cancer may be of a known or unknown origin.

### Inhibitory Agents

It is envisaged that the agent may be any that reduces, inhibits or disrupts an expression and/or an activity of one or more of TERF2IP, SAMHD1, CCDC93, CHTF18, ETAA1, ZMAT1, SIVA1, FZR1 and SUB1. The agent may be a direct inhibitor of the expression and/or activity of TERF2IP, SAMHD1, CCDC93, CHTF18, ETAA1, ZMAT1, SIVA1, FZR1 and/or SUB1 or an indirect inhibitor of the expression and/or activity of TERF2IP, SAMHD1, CCDC93, CHTF18, ETAA1, ZMAT1, SIVA1, FZR1 and/or SUB1. For example, the agent may bind to one of these mediators of an ALT mechanism to inhibit its function by changing its conformation or by affecting a binding site thereon. In other examples, the agent may bind to an encoding nucleic acid (e.g., mRNA, such as those provided herein) of TERF2IP, SAMHD1, CCDC93, CHTF18, ETAA1, ZMAT1, SIVA1, FZR1 and/or SUB1 to inhibit its expression.

The term “inhibitor” as used herein refers to a molecule having the ability to inhibit a biological function and/or expression of a target polypeptide, such as TERF2IP, SAMHD1, CCDC93, CHTF18, ETAA1, ZMAT1, SIVA1, FZR1 and/or SUB1. The term “selective inhibition” or “selectively inhibit” refers to the agent's ability to preferentially reduce the target polypeptide's expression, signalling activity, such as a enzymatic activity, kinase activity or binding activity, as compared to off-target signalling activity, via direct or indirect interaction with the target polypeptide or an encoding nucleic acid.

With respect to the agent described for the present disclosure, it will be appreciated that this refers to a compound or a substance that acts against or blocks, at least in part, the expression, physiological/biological function or activity, such as the enzymatic, kinase or signalling activity, of TERF2IP, SAMHD1, CCDC93, CHTF18, ETAA1, ZMAT1, SIVA1, FZR1 and/or SUB1. Suitably, the agent binds or physically interacts with TERF2IP, SAMHD1, CCDC93, CHTF18, ETAA1, ZMAT1, SIVA1, FZR1 and/or SUB1 or an encoding nucleic acid thereof. In some examples, the inhibitor is an antigen-binding molecule, such as an antibody or antigen-binding fragment thereof, an inhibitory nucleic acid molecule against the expression of the target protein, an inhibitory polypeptide, such as dominant-negative polypeptides or aptamers, or a small molecule inhibitor.

Suitably, the agent is a genetic inhibitor of TERF2IP, SAMHD1, CCDC93, CHTF18, ETAA1, ZMAT1, SIVA1, FZR1 or SUB1. Methods of designing suitable genetic inhibitors are known in the art. Suitable examples of genetic inhibitors include, but are not limited to, DNA (gDNA, cDNA), RNA (sense RNAs, antisense RNAs, mRNAs, tRNAs, rRNAs, small interfering RNAs (siRNAs), short hairpin RNAs (ShRNAs), micro RNAs (miRNAs), small nucleolar RNAs (SnoRNAs), small nuclear RNAs (snRNAs), ribozymes, aptamers, DNAzymes, antisense oliogonucleotides, vectors, plasmids, targeted nuclease (e.g., CRISPR/Cas-associated nuclease, ZEN, TALEN or meganuclease that recognises a target sequence), other ribonuclease-type complexes, and mixtures thereof. The targeted nuclease may be site-specific (e.g., ZFN or TALEN) or may be expressed with one or more targeting sequences that target the nuclease to a gene of interest (e.g., CRISPR/Cas). The targeted nuclease may inactivate the target gene encoding TERF2IP, SAMHD1, CCDC93, CHTF18, ETAA1, ZMAT1, SIVA1, FZR1 or SUB1 in one or more cells of the individual, for example, by introducing one or more mutations that prevent the expression of an active polypeptide thereof. The targeted nuclease may be specifically targeted to cancer cells by conventional techniques, including cell targeted delivery vehicles, such as viral vectors that express a ligand for a specific cell type; direct administration of the targetable nuclease to a tumour (e.g., by injection; or the expression of the targetable nuclease from heterologous nucleic acid selectively in cancer cells, for example using a tissue specific promoter). The gene sequences of TERF2IP, SAMHD1, CCDC93, CHTF18, ETAA1, ZMAT1, SIVA1, FZR1 and SUB1 are publicly available and can be used to design suitable genetic inhibitors by methods known in the art. Reference nucleotide sequences of TERF2IP, SAMHD1, CCDC93, CHTF18, ETAA1, ZMAT1, SIVA1, FZR1 and SUB1 are provided in SEQ ID NOs 2, 4, 6, 8, 10, 12, 14 and 16 respectively.

In particular examples, the agent is or comprises an inhibitory nucleic acid molecule. By “inhibitory nucleic acid molecule” is meant a polynucleotide that disrupts the expression of a target nucleic acid molecule or an encoded polypeptide. In such examples, the inhibitory nucleic acid molecule suitably comprises a sequence complementary to an TERF2IP-, SAMHD1-, CCDC93-, CHTF18-, ETAA1-, ZMAT1-, SIVA1-, FZR1- or SUB1-encoding nucleic acid sequence, such as one that encodes the protein sequences of SEQ ID NOs: 1, 3, 5, 7, 9, 11, 13, 15 or 46. Exemplary inhibitory nucleic acid molecules include a small interfering RNA (siRNA; including divalent siRNAs), a microRNA-adapted shRNA, a short hairpin RNA (shRNA), a hairpin siRNA, a precursor microRNA (pre-miRNA), pri-miRNA, a microRNA (miRNA), ribozymes or an antisense oligonucleotide.

The term “nucleic acid” as used herein designates single- or double-stranded DNA and RNA. DNA includes genomic DNA and cDNA, as well as oligonucleotides (e.g., anti-sense oligonucleotides). RNA includes mRNA, RNA, RNAi, siRNA, shRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as modified purines (for example inosine, methylinosine and methyladenosine) and modified pyrimidines (for example thiouridine and methylcytosine). It is further contemplated that one or more T nucleobases of the nucleic acids described herein may be replaced with a U nucleobase without substantially altering the functionality thereof. Similarly, one or more U nucleobases of the nucleic acids described herein may be replaced with a T nucleobase without substantially altering the functionality thereof.

Examples of suitable genetic inhibitors and inhibitory nucleic acids are described herein in the experimental examples. Thus, the genetic inhibitors may comprise siRNA inhibitors.

Accordingly, in one form, the present disclosure provides a genetic inhibitor comprising, consisting of or consisting essentially of the nucleic acid sequence selected from the group consisting of SEQ ID NOs: 20 to 45, or a fragment, variant or derivative thereof.

Suitably, the genetic inhibitor is or comprises an siRNA or an siRNA molecule. For such examples, the siRNA molecule may comprise a pair of single stranded nucleic acid molecules that are substantially complementary to or hybridize to each other to form a double stranded siRNA molecule. In some examples, the siRNA molecule comprises the nucleotide sequences of SEQ ID NOs: 20 and 21. In some examples, the siRNA molecule comprises the nucleotide sequences of SEQ ID NOs: 22 and 23. In some examples, the siRNA molecule comprises the nucleotide sequences of SEQ ID NOs: 24 and 25. In some examples, the siRNA molecule comprises the nucleotide sequences of SEQ ID NOs: 26 and 27. In some examples, the siRNA molecule comprises the nucleotide sequences of SEQ ID NOs: 28 and 29. In some examples, the siRNA molecule comprises the nucleotide sequences of SEQ ID NOs: 30 and 31. In some examples, the siRNA molecule comprises the nucleotide sequences of SEQ ID NOs: 32 and 33. In some examples, the siRNA molecule comprises the nucleotide sequences of SEQ ID NOs: 34 and 35. In some examples, the siRNA molecule comprises the nucleotide sequences of SEQ ID NOs: 36 and 37. In some examples, the siRNA molecule comprises the nucleotide sequences of SEQ ID NOS: 38 and 39. In some examples, the siRNA molecule comprises the nucleotide sequences of SEQ ID NOs: 40 and 41. In some examples, the siRNA molecule comprises the nucleotide sequences of SEQ ID NOs: 42 and 43. In some examples, the siRNA molecule comprises the nucleotide sequences of SEQ ID NOs: 44 and 45.

As used herein, a nucleic acid “variant” shares a definable nucleotide sequence relationship with a reference nucleic acid sequence (e.g., SEQ ID NOs: 2, 4, 6, 8, 10, 12, 14, 16, 20 to 45 and 47). The “variant” nucleic acid may have one or a plurality of nucleic acids of the reference nucleic acid sequence deleted or substituted by different nucleic acids. It is well understood in the art that some nucleic acids of a DNA/RNA-based binding or recognition site may be substituted or deleted without changing (or only having minimal change to) the affinity of the target polynucleotide (e.g., genetic inhibitor) therefor. Suitably, nucleic acid variants share at least 60% or 65%, 66%, 67%, 68%, 69%, preferably at least 70%, 71%, 72%, 73%, 74% or 75%, more particularly at least 80%, 81%, 82%, 83%, 84%, 85%, 86%, 87%, 88% or 89%, and even more particularly at least 90%, 91%, 92%, 93%, 94%, 95%, 96%, 97%, 98% or 99% nucleotide sequence identity with an isolated nucleic acid of the present disclosure (e.g., SEQ ID NOs: 2, 4, 6, 8, 10, 12, 14, 16, 20 to 45 and 47). Percent sequence identity may be determined by any method known in the art, such as that described herein.

Also contemplated herein are nucleic acid fragments. A “fragment” is a segment, domain, portion or region of a nucleic acid, which respectively constitutes less than 100% of the nucleotide sequence. A non-limiting example is an amplification product or a primer or probe. In particular examples, a nucleic acid fragment may comprise, for example, at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80 (inclusive of any range therein) contiguous nucleotides of said nucleic acid (e.g., SEQ ID NOs: 2, 4, 6, 8, 10, 12, 14, 16, 20 to 45 and 47 and more particularly SEQ ID NOs: 20 to 45).

Further contemplated herein are nucleic acid derivatives, inclusive of genetic inhibitor derivatives. Such nucleic acid derivatives may include, for example, one or more modifications and/or conjugates as described herein.

By way of example, the genetic inhibitor may be conjugated to one or more moieties or groups which enhance the activity, cellular distribution or cellular uptake thereof. These moieties or groups may be covalently bound to functional groups such as primary or secondary hydroxyl groups. Exemplary moieties or groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins and dyes/labels (e.g., Cy5 dye to determine cellular uptake and/or localisation of the genetic inhibitor).

Genetic inhibitors of the present disclosure may have nucleobase (“base”) and/or internucleotide or backbone modifications or substitutions. Such modifications can advantageously increase the binding specificity of the genetic inhibitor for the target region of a target mRNA molecule, minimise or reduce digestion by nucleases and/or reduce or minimise the immunogenicity or antigenicity of the genetic inhibitor. The genetic inhibitors can employ a variety of chemistries. Examples of oligomer chemistries include, without limitation, phosphoramidate morpholino oligomers and phosphorodiamidate morpholino oligomers (PMO), phosphorothioate modified oligomers, 2′ O-methyl modified oligomers, peptide nucleic acid (PNA), locked nucleic acid (LNA), phosphorothioate oligomers, 2′ O-MOE modified oligomers, 2′-fluoro-modified oligomer, 2′O,4′C-ethylene-bridged nucleic acids (ENAs), tricyclo-DNAs, tricyclo-DNA phosphorothioate nucleotides, 2′-O-[2-(N-methylcarbamoyl)ethyl] modified oligomers, morpholino oligomers, peptide-conjugated phosphoramidate morpholino oligomers (PPMO), phosphorodiamidate morpholino oligomers having a phosphorous atom with (i) a covalent bonds to the nitrogen atom of a morpholino ring, and (ii) a second covalent bond to a (1,4-piperazin)-1-yl substituent or to a substituted (1,4-piperazin)-1-yl (PMOplus), and phosphorodiamidate morpholino oligomers having a phosphorus atom with (i) a covalent bond to the nitrogen atom of a morpholino ring and (ii) a second covalent bond to the ring nitrogen of a 4-aminopiperdin-1-yl (i.e., APN) or a derivative of 4-aminopiperdin-1-yl (PMO-X) chemistries, including combinations of any of the foregoing.

According to other examples, the agent may be a small molecule, a peptide or a protein. In some examples, the agent is a small molecule. In other examples, the agent is a peptide or a protein, such as an aptamer, an inactivated protein or a dominant negative protein. Such small molecules, proteins and peptides are suitably capable of binding, directly or indirectly, to TERF2IP, SAMHD1, CCDC93, CHTF18, ETAA1, ZMAT1, SIVA1, FZR1 or SUB1 and inhibiting an activity thereof.

In particular examples, the agent is a Vpx protein, or a functional fragment, variant or derivative thereof. In such examples, the agent is suitably an inhibitor of SAMHD1. To this end, it has been previously demonstrated that the Vpx protein is a potent inhibitor of SAMHD1 expression (see Kim et al., J Biol Chem. 2012 Jun. 22; 287 (26): 21570-21574).

By “protein” is meant an amino acid polymer. The amino acids may be natural or non-natural amino acids, D- or L-amino acids as are well understood in the art. As would be appreciated by the skilled person, the term “protein” also includes within its scope phosphorylated forms of a protein (i.e., a phosphoprotein) and/or glycosylated forms of a protein (i.e. a glycoprotein). A “peptide” is a protein having no more than fifty (50) amino acids. A “polypeptide” is a protein having more than fifty (50) amino acids.

Also provided are protein “variants” such as naturally occurring (e.g. allelic variants) and orthologs of a Vpx protein. Suitably, protein variants share at least 70% or 75%, particularly at least 80% or 85% or more particularly at least 90%, 91%, 92%, 93%, 94%, 95%, 96%, 97%, 98% or 99% sequence identity with an amino acid sequence of a Vpx protein disclosed herein or known in the art (e.g., SEQ ID NOs: 17-19).

Also provided are protein fragments, inclusive of peptide fragments that comprise less than 100% of an entire amino acid sequence. In particular examples, a protein fragment may comprise, for example, at least 10, 15, 20, 25, 30 35, 40, 45, 50, 55, 60, 65, 70, 75, 80, 85, 90, 95, 100, 105, 110 and 111 contiguous amino acids of said protein (e.g., SEQ ID NOs: 17-19).

As used herein, “derivatives” refers to proteins, inclusive of fragments or variants thereof, that have been altered, for example by conjugation or complexing with other chemical moieties, such as by post-translational modification (e.g., phosphorylation, acetylation and the like), modification of glycosylation (e.g., adding, removing or altering glycosylation), lipidation and/or inclusion of additional amino acid sequences as would be understood in the art.

The Vpx protein is a virion-associated protein encoded by Human Immunodeficiency Virus type 2 HIV (HIV-2) and most Simian Immunodeficiency Virus (SIV). Interestingly, the Vpx protein is absent from HIV-1. The Vpx protein is similar in structure to the protein Vpr that is carried by SIV and HIV-2, as well as HIV-1. The Vpx protein is one of the five accessory proteins (Vif, Vpx, Vpr, Vpu, and Nef) carried by lentiviruses that enhances viral replication by inhibiting host antiviral factors.

The Vpx protein, or fragment, variant or derivative thereof, for use according to the present disclosure is suitably a functional Vpx protein. By “functional Vpx protein” is meant a protein able to bind (directly or indirectly) to SAMHD1, and to trigger SAMHD1 degradation in a cell. Determining whether a Vpx protein, or fragment, variant or derivative thereof, is functional may be assessed by any method or means known in the art.

A variant of a Vpx protein is for example a functional Vpx protein that has a sequence at least 80% identical to said Vpx protein, more particularly at least 85% identical to said Vpx protein, even more particularly at least 90% identical to said Vpx protein, still more particularly at least 91%, at least 92%, at least 93%, at least 94%, at least 95%, at least 96%, at least 97%, at least 98% or at least 99% identical to said Vpx protein (e.g., SEQ ID NO: 17, 18 or 19).

An exemplary Vpx protein comprises, consists of or consists essentially of an amino acid sequence at least about 80% (e.g., at least about 80%, 81%, 82%, 83%, 84%, 85%, 86%, 87%, 88%, 89%, 90%, 91%, 92%, 93%, 94%, 95%, 96%, 97%, 98%, 99%, 100% or any range therein) identical to an amino acid sequence selected from the group consisting of SEQ ID NOs: 17 to 19.

An exemplary HIV-2 Vpx protein is set forth in SEQ ID NO: 17 below, which corresponds to the sequence of Ghana-1 HIV-2 of accession number P18045 in the UniProtKB database.

SEQ ID NO: 17
MTDPRERVPPGNSGEETIGEAFEWLDRTIEALNREAVNHLPRELIFQVWQ
RSWRYWHDDQGMSPSYTKYRYLCLMQKAVFIHFKRGCTCLGGGHGPGGWR
SGPPPPPPPGLV

Another example of a HIV-2 Vpx protein is set forth in SEQ ID NO: 18 below, which corresponds to the sequence of Rod9 HIV-2 of accession number APS24116.1 in the Genbank database.

SEQ ID NO: 18
MTDPRETVPPGNSGEETIGEAFAWLNRTVEAINREAVNHLPRELIFQVWQ
RSWRYWHDEQGMSESYTKYRYLCIMQRAVYMHVRKGCTCLGRGHGPGGWR
PGPPPPPPPGLV

An example of an SIVsmm (Sooty Mangabey Monkey SIV) Vpx protein is set forth in SEQ ID NO: 22, which corresponds to the sequence of accession number P19508.1 in the UniProtKB database.

SEQ ID NO: 19
MSDPRERIPPGNSGEETIGEAFDWLDRTVEEINRAAVNHLPRELIFQVWR
RSWEYWHDEMGMSVSYTKYRYLCLIQKAMFMHCKKGCRCLGGEHGAGGWR
PGPPPPPPPGLA

Terms used generally herein to describe sequence relationships between respective nucleotides or polypeptides include “sequence identity”, “percentage of sequence identity” and “substantial identity”. Because respective nucleotides or polypeptides may each comprise (i) only one or more portions of a complete nucleotide or polypeptide sequence that are shared by the nucleotides or amino acids, and (ii) one or more portions which are divergent between the nucleotides or amino acids, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, WI, USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) or amino acid residue occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, California, USA).

According to other examples, the agent is an antigen-binding molecule. Suitably, the antigen-binding molecule is an immunoglobulin. More particularly, the agent can be an antibody or antibody fragment, such as a monoclonal antibody, a multi-specific antibody (e.g., a bi-specific antibody), a single-chain Fv (scFv), an Fab fragment, an Fab′ fragment, an F(ab′) fragment, an F(ab′)₂ fragment, a diabody, an intrabody and a synthetic antibody, that binds to a specific region of TERF2IP, SAMHD1, CCDC93, CHTF18, ETAA1, ZMAT1, SIVA1, FZR1 or SUB1. In particular examples, the agent described herein is a monoclonal antibody.

The term “immunoglobulin” will be understood to include any binding agent comprising an immunoglobulin domain. Exemplary immunoglobulins are antibodies. Additional proteins encompassed by the term “immunoglobulin” include domain antibodies, camelid antibodies and antibodies from cartilaginous fish (i.e., immunoglobulin new antigen receptors (IgNARs)). Generally, camelid antibodies and IgNARs comprise a V_{H} domain, however lack a V_{L} domain and are often referred to as heavy chain immunoglobulins, single domain antibodies or nanobodies.

The term “antibody” is used in the context of the present disclosure to refer to immunoglobulin molecules immunologically reactive with a particular antigen and includes both polyclonal and monoclonal antibodies. The term also includes native and genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rlgG as discussed in Pierce Catalogue and Handbook, 1994-1995 (Pierce Chemical Co., Rockford, Ill.); Kuby, J., Immunology, 3^{rd} Ed., W.H. Freeman & Co., New York (1998). The term antibody also includes multispecific molecules, such as bivalent or bispecific molecules. Examples of bivalent and bispecific molecules are described in Kostelny et al. (1992) J Immunol 148:1547; Pack and Pluckthun (1992) Biochemistry 31:1579; Hollinger et al., 1993, supra, Gruber et al. (1994) J. Immunol.: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An “antibody fragment” or “antigen binding fragment” of an antibody comprises one or more variable regions of an intact antibody. Examples of antibody fragments include Fab, Fab′, F(ab′)₂ and Fv fragments; diabodies; triabodies; tetrabodies; dimerisation-activated demibodies (e.g., WO/2007/062466); linear antibodies; single-chain antibody molecules and multispecific antibodies formed from antibody fragments. For example, the term antigen binding fragment may be used to refer to recombinant single chain Fv fragments (scFv), as well as divalent (di-scFv) and trivalent (tri-scFV) forms thereof.

Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

The terms “full-length antibody”, “intact antibody” or “whole antibody” are used interchangeably herein to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3.

“Framework regions” (Syn. FR) are those variable domain residues other than the CDR residues.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy C_{H}1, a linker, a C_{H}2 and a C_{H}3. In a heavy chain, a constant region comprises a Fc. In a light chain, a constant region generally comprise one constant domain (a CL1).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Exemplary heavy chain constant regions are gamma 1 (IgG1), gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of γ, α or δ heavy chain comprises two constant domains.

Generally, antibodies of the present disclosure bind to or conjugate with an isolated protein, fragment, variant, or derivative of TERF2IP, SAMHD1, CCDC93, CHTF18, ETAA1, ZMAT1, SIVA1, FZR1 or SUB1. For example, the antibodies may be polyclonal antibodies. Such antibodies may be prepared for example by injecting an isolated protein, fragment, variant or derivative of TERF2IP, SAMHD1, CCDC93, CHTF18, ETAA1, ZMAT1, SIVA1, FZR1 or SUB1 into a production species, which may include mice or rabbits, to obtain polyclonal antisera. Methods of producing polyclonal antibodies are well known to those skilled in the art. Exemplary protocols which may be used are described for example in Coligan et al, CURRENT PROTOCOLS IN IMMUNOLOGY, supra, and in Harlow & Lane, 1988, supra.

Monoclonal antibodies may be produced using the standard method as for example, described in an article by Kohler & Milstein, 1975, Nature 256, 495, which is herein incorporated by reference, or by more recent modifications thereof as for example, described in Coligan et al, CURRENT PROTOCOLS IN IMMUNOLOGY, supra by immortalizing spleen or other antibody producing cells derived from a production species which has been inoculated with the isolated TERF2IP, SAMHD1, CCDC93, CHTF18, ETAA1, ZMAT1, SIVA1, FZR1 or SUB1 and/or fragments, variants and/or derivatives thereof.

It will be appreciated that inhibition by the various inhibitors described herein need not be absolute to elicit a biological effect, such as treatment of an ALT-dependent cancer in the subject. Accordingly, inhibition by an agent provided herein can be partial (e.g., an expression, a function and/or an activity, of TERF2IP, SAMHD1, CCDC93, CHTF18, ETAA1, ZMAT1, SIVA1, FZR1 and/or SUB1 is reduced by about 20%, 30%, 40%, 50%, 60% or 70%, 80%, 90%, 95%, 96%, 97%, 98% and 99%, including any intermediate value therebetween) in the presence of the agent as described herein, such as when compared to a level of expression, function and/or activity thereof in the absence of the agent.

### TERF2IP

Suitably, the agent described herein reduces an expression and/or an activity of TERF2IP.

TERF2IP (Telomeric repeat-binding factor 2-interacting protein 1) acts as a regulator of telomere function and a transcription regulator, so as to be involved in the regulation of telomere length and protection as a component of the shelterin complex. The nucleotide and protein sequences of TERF2IP are publicly available (e.g., Q9NYB0, ENST00000300086.5 and NM_018975.3). An exemplary amino acid sequence is set forth in SEQ ID NO: 1. Thus, the TERF2IP amino acid sequence may be a protein which is at least 70%, 75%, 80%, 85%, 90%, 95%, 96%, 97%, 98%, 99%, 99.5% or 100% identical to SEQ ID NO:1 or a fragment or derivative thereof. An exemplary encoding nucleotide sequence (i.e., mRNA transcript) is set forth in SEQ ID NO: 2. Thus, the TERF2IP nucleotide sequence may be a nucleic acid which is at least 70%, 75%, 80%, 85%, 90%, 95%, 96%, 97%, 98%, 99%, 99.5% or 100% identical to SEQ ID NO:2 or a fragment or derivative thereof.

TERF2IP protein
(SEQ ID NO: 1)
MAEAMDLGKDPNGPTHSSTLFVRDDGSSMSFYVRPSPAKRRLSTLILHGG
GTVCRVQEPGAVLLAQPGEALAEASGDFISTQYILDCVERNERLELEAYR
LGPASAADTGSEAKPGALAEGAAEPEPQRHAGRIAFTDADDVAILTYVKE
NARSPSSVTGNALWKAMEKSSLTQHSWQSLKDRYLKHLRGQEHKYLLGDA
PVSPSSQKLKRKAEEDPEAADSGEPQNKRTPDLPEEEYVKEEIQENEEAV
KKMLVEATREFEEVVVDESPPDFEIHITMCDDDPPTPEEDSETQPDEEEE
EEEEKVSQPEVGAAIKIIRQLMEKFNLDLSTVTQAFLKNSGELEATSAFL
ASGQRADGYPIWSRQDDIDLQKDDEDTREALVKKFGAQNVARRIEFRKK
TERF2IP nucleic acid
(SEQ ID NO: 2)
ACTCTCGATGCGCCGCGGCTGCTGCTGCGCAGGCCCAGTGCTGCGCTTCG
CGGCAGAGGCGTCTGCGGTGACAGCTCAGTCAGTTGAGCTCTGTGTGCCA
GGCGCTCGCGAGGGGGTAGCTCTTCTAGTAGTGCTCGGCGTCAGACATGG
CGGAGGCGATGGATTTGGGCAAAGACCCCAACGGGCCCACCCATTCCTCG
ACTCTGTTCGTGAGGGACGACGGCAGCTCCATGTCCTTCTACGTGCGGCC
CAGCCCGGCCAAGCGTCGGCTGTCGACGCTCATCCTGCACGGCGGCGGCA
CCGTGTGCCGAGTGCAGGAGCCCGGGGCCGTGCTGCTGGCCCAGCCCGGG
GAGGCGCTGGCCGAGGCCTCGGGTGATTTCATCTCCACGCAGTACATCCT
GGACTGCGTGGAGCGCAACGAGAGGCTGGAGCTGGAGGCCTATCGGCTGG
GCCCCGCCTCGGCGGCGGACACCGGCTCGGAAGCAAAGCCCGGGGCCCTG
GCCGAGGGCGCCGCGGAGCCGGAGCCGCAGCGGCACGCCGGGCGGATCGC
CTTCACGGATGCGGACGACGTAGCCATCCTTACCTACGTGAAGGAAAATG
CCCGCTCGCCCAGCTCCGTCACCGGTAACGCCTTGTGGAAAGCGATGGAG
AAGAGCTCGCTCACGCAGCACTCGTGGCAGTCCCTGAAGGACCGCTACCT
CAAGCACCTGCGGGGCCAGGAGCATAAGTACCTGCTGGGGGACGCGCCGG
TGAGCCCCTCCTCCCAGAAGCTCAAGCGGAAGGCGGAGGAGGACCCGGAG
GCCGCGGATAGCGGGGAACCACAGAATAAGAGAACTCCAGATTTGCCTGA
AGAAGAGTATGTGAAGGAAGAAATCCAGGAGAATGAAGAAGCAGTCAAAA
AGATGCTTGTGGAAGCCACCCGGGAGTTTGAGGAGGTTGTGGTGGATGAG
AGCCCTCCTGATTTTGAAATACATATAACTATGTGTGATGATGATCCACC
CACACCTGAGGAAGACTCAGAAACACAGCCTGATGAGGAGGAAGAAGAAG
AAGAAGAAAAAGTTTCTCAACCAGAGGTGGGAGCTGCCATTAAGATCATT
CGGCAGTTAATGGAGAAGTTTAACTTGGATCTATCAACAGTTACACAGGC
CTTCCTAAAAAATAGTGGTGAGCTGGAGGCTACTTCCGCCTTCTTAGCGT
CTGGTCAGAGAGCTGATGGATATCCCATTTGGTCCCGACAAGATGACATA
GATTTGCAAAAAGATGATGAGGATACCAGAGAGGCATTGGTCAAAAAATT
TGGTGCTCAGAATGTAGCTCGGAGGATTGAATTTCGAAAGAAATAATTGG
CAAGATAATGAGAAAAGAAAAAAGTCATGGTAGGTGAGGTGGTTAAAAAA
AATTGTGACCAATGAACTTTAGAGAGTTCTTGCATTGGAACTGGCACTTA
TTTTCTGACCATCGCTGCTGTTGCTCTGTGAGTCCTAGATTTTTGTAGCC
AAGCAGAGTTGTAGAGGGGGATAAAAAGAAAAGAAATTGGATGTATTTAC
AGCTGTCCTTGAACAAGTATCAATGTGTTTATGAAAGGAAGATCTAAATC
AGACAGGAGTTGGTCTACATAGTAGTAATCCATTGTTGGAATGGAACCCT
TGCTATAGTAGTGACAAAGTGAAAGGAAATTTAGGAGGCATAGGCCATTT
CAGGCAGCATAAGTAATCTCCTGTCCTTTGGCAGAAGCTCCTTTAGATTG
GGATAGATTCCAAATAAAGAATCTAGAAATAGGAGAAGATTTAATTATGA
GGCCTTGAACACGGATTATCCCCAAACCCTTGTCATTTCCCCCAGTGAGC
TCTGATTTCTAGACTGCTTTGAAAATGCTGTATTCATTTTGCTAACTTAG
TATTTGGGTACCCTGCTCTTTGGCTGTTCTTTTTTTGGAGCCCTTCTCAG
TCAAGTCTGCCGGATGTCTTTCTTTACCTACCCCTCAGTTTTCCTTAAAA
CGCGCACACAACTCTAGAGAGTGTTAAGAATAATGTTACTTGGTTAATGT
GTTATTTATTGAGTATTGTTTGTGCTAAGCATTGTGTTAGATTTAAAAAA
TTAGTGGATTGACTCCACTTTGTTGTGTTGTTTTCATTGTTGAAAATAAA
TATAACTTTGTATTCGAGTCTCGTCAAAAAAAAAAAAAAAAAAAAA

### SAMHD1

Suitably, the agent described herein reduces an expression and/or an activity of SAMHD1.

SAMHD1 (SAM and HD Domain Containing Deoxynucleoside Triphosphate Triphosphohydrolase 1) acts both as a host restriction factor involved in defense responses to viral infections and as a regulator of DNA end resection at stalled replication forks. The nucleotide and protein sequences of SAMHD1 are publicly available (e.g., Q9Y3Z3, ENST00000646673.2 and NM_015474.3). An exemplary amino acid sequence is set forth in SEQ ID NO: 3. Thus, the SAMHD1 amino acid sequence may be a protein which is at least 70%, 75%, 80%, 85%, 90%, 95%, 96%, 97%, 98%, 99%, 99.5% or 100% identical to SEQ ID NO:3 or a fragment or derivative thereof. An exemplary encoding nucleotide sequence (i.e., mRNA transcript) is set forth in SEQ ID NO: 4. Thus, the SAMHD1 nucleotide sequence may be a nucleic acid which is at least 70%, 75%, 80%, 85%, 90%, 95%, 96%, 97%, 98%, 99%, 99.5% or 100% identical to SEQ ID NO:4 or a fragment or derivative thereof.

SAMHD1 protein
(SEQ ID NO: 3)
MQRADSEQPSKRPRCDDSPRTPSNTPSAEADWSPGLELHPDYKTWGPEQV
CSFLRRGGFEEPVLLKNIRENEITGALLPCLDESRFENLGVSSLGERKKL
LSYIQRLVQIHVDTMKVINDPIHGHIELHPLLVRIIDTPQFQRLRYIKQL
GGGYYVFPGASHNRFEHSLGVGYLAGCLVHALGEKQPELQISERDVLCVQ
IAGLCHDLGHGPFSHMEDGRFIPLARPEVKWTHEQGSVMMFEHLINSNGI
KPVMEQYGLIPEEDICFIKEQIVGPLESPVEDSLWPYKGRPENKSFLYEI
VSNKRNGIDVDKWDYFARDCHHLGIQNNFDYKRFIKFARVCEVDNELRIC
ARDKEVGNLYDMFHTRNSLHRRAYQHKVGNIIDTMITDAFLKADDYIEIT
GAGGKKYRISTAIDDMEAYTKLTDNIFLEILYSTDPKLKDAREILKQIEY
RNLFKYVGETQPTGQIKIKREDYESLPKEVASAKPKVLLDVKLKAEDFIV
DVINMDYGMQEKNPIDHVSFYCKTAPNRAIRITKNQVSQLLPEKFAEQLI
RVYCKKVDRKSLYAARQYFVQWCADRNFTKPQDGDVIAPLITPQKKEWND
STSVQNPTRLREASKSRVQLFKDDPM
SAMHD1 nucleic acid
(SEQ ID NO: 4)
ATTGCGCCTGCGCAGGGAGCCCAAGGCAAGAGCCGCTAGGCTGCCCTGCC
CGAAGGGCTCAACTGTCAGTGAGCCTGCGCAGGAGGCCAATAGGCTGCCA
ATACTCCTTGGACTCCCCGCCAGGGCCCTGCTGTCAGTGCGCCTGCGCGC
GGGTCCGGCGCCGAGGTTCTTGACTGCTGTGCCGGACGCCAGGTGTAGCC
ATGCAGCGAGCCGATTCCGAGCAGCCCTCCAAGCGTCCCCGTTGCGATGA
CAGCCCGAGAACCCCCTCAAACACCCCTTCCGCAGAGGCAGACTGGTCCC
CGGGCCTGGAACTCCATCCCGACTACAAGACATGGGGTCCGGAGCAGGTG
TGCTCCTTCCTCAGGCGCGGTGGCTTTGAAGAGCCGGTGCTGCTGAAGAA
CATCCGAGAAAATGAAATCACAGGCGCATTACTGCCTTGTCTTGATGAGT
CTCGTTTTGAAAATCTTGGAGTAAGTTCCTTGGGGGAGAGGAAGAAGCTG
CTTAGTTATATCCAGCGATTGGTTCAAATCCACGTTGATACAATGAAGGT
AATTAATGATCCTATCCATGGCCACATTGAGCTCCACCCTCTCCTCGTCC
GAATCATTGATACACCTCAATTTCAACGTCTTCGATACATCAAACAGCTG
GGAGGTGGTTACTATGTTTTTCCAGGAGCTTCACACAATCGATTTGAGCA
TAGTCTAGGGGTGGGGTATCTAGCAGGATGTCTAGTTCACGCACTGGGTG
AAAAACAACCAGAGCTGCAGATAAGTGAACGAGATGTTCTCTGTGTTCAG
ATTGCTGGACTTTGTCATGATCTCGGTCATGGGCCATTTTCTCACATGTT
TGATGGACGATTTATTCCACTTGCTCGCCCGGAGGTGAAATGGACGCATG
AACAAGGCTCAGTTATGATGTTTGAGCACCTTATTAATTCTAATGGAATT
AAGCCTGTCATGGAACAATATGGTCTCATCCCTGAAGAAGATATTTGCTT
TATAAAGGAACAAATTGTAGGACCACTTGAATCACCTGTCGAAGATTCAT
TGTGGCCATATAAAGGGCGTCCTGAAAACAAAAGCTTCCTTTATGAGATA
GTATCTAATAAAAGAAATGGCATTGATGTGGACAAATGGGATTATTTTGC
CAGGGACTGCCATCATCTTGGAATCCAAAATAATTTTGATTACAAGCGCT
TTATTAAGTTTGCCCGTGTCTGTGAAGTAGACAATGAGTTGCGTATTTGT
GCTAGAGATAAGGAAGTTGGAAATCTGTATGACATGTTCCACACTCGCAA
CTCTTTACACCGTAGAGCTTATCAACACAAAGTTGGCAACATTATTGATA
CAATGATTACAGATGCTTTCCTCAAAGCAGATGACTACATAGAGATTACA
GGTGCTGGAGGAAAAAAGTATCGCATTTCTACAGCAATTGACGACATGGA
AGCCTATACTAAGCTGACAGATAACATTTTTCTGGAGATTTTATACTCTA
CTGATCCCAAATTGAAAGACGCACGAGAGATTTTAAAACAAATTGAATAC
CGTAATCTATTCAAGTATGTGGGTGAGACGCAGCCAACAGGACAAATAAA
GATTAAAAGGGAGGACTATGAATCTCTTCCAAAAGAGGTTGCCAGTGCTA
AACCCAAAGTATTGCTAGACGTGAAACTGAAGGCTGAAGATTTTATAGTG
GATGTTATCAACATGGATTATGGAATGCAAGAAAAGAATCCAATTGATCA
TGTTAGCTTCTATTGTAAGACTGCCCCCAACAGAGCAATCAGGATTACTA
AAAACCAGGTTTCACAACTTCTGCCAGAGAAATTTGCAGAGCAGCTGATT
CGAGTATATTGTAAGAAGGTGGACAGAAAGAGTTTGTATGCCGCAAGACA
ATATTTTGTTCAGTGGTGTGCAGACAGAAATTTCACCAAGCCGCAGGATG
GCGATGTTATAGCCCCACTCATAACACCTCAAAAAAAGGAATGGAACGAC
AGTACTTCAGTCCAAAATCCAACTCGCCTCCGAGAAGCATCCAAAAGCAG
AGTCCAGCTTTTTAAAGATGACCCAATGTGAATGTCTGTAGTCAGTTGTT
TACAAACTCCCTCTCCTGCACAATTCATTTAGAGGCTTCAATCATAGAAT
TCTGCAAATTAATGACAACTCATGCTTTAATTTTGTATTTTGAATGTACA
CGCATGCTGAAGCTAAGTAACTTTTAATCAAAGAAATAAGATGGTATTAG
GCAAATCTTACTATACTATGAAAAGCATTACCTTGCCTATTTTTAATATT
ATTAAAGCCTTTCTCCTTCAGTAGTCTATTTTCTTAGAATAACAACTCTT
TTATCTATTCTGAACTCTATTTTTTTTCTTTTTTAAGAGACAAGGTTTTG
CTCTGTTGCCCAGCTTGGACTCGAACTTTCCTGGGCTCAAGCGACCCTCC
TGCCTCAGCCCCCCAAGTAGCTGGGACTAAAGTCATGTGCCACCACACCC
AGCTTACTCTGAACTTTTATGACAGATGATTGTTTTTTGTTTTTAATGTA
GAAATGAGACAAGGGTACAAATTGGAACTAGAAATTGACATTGTCATTGA
CAAACATGGCTAAAAACAAAACATCAAATCCTGCCCCCGTGAAGAGTTCC
CTGTCACCTCAAGTTTGAGGATAGTCCTCTAAGAGTGACCTAAGCATAAG
TGAAAGACACCTCCCCTCACCCTTCTAGCCCCCTACAAGGTGCCAGGTTG
GGGTAAAGAGTTGGAGATGATGGCCAGGAGTGGCCTCCAACACGCTGGTG
AGAGGCCTGATTAGGTTTTGGGGAAGATCTGAGAGCTCTGGCCTCTTCGT
GAGTGGAACATAAAGCCGCCTCTTGTTGGGAGATCCTACCCCAGTGACAG
AGGAATCCCCCAAACTAGGCTGTGCCCTGGCTCCGTGGCGGCTCCAGACC
CGGGTAGTGCCTTTGTCCCCTGAATACTCACTCCCCCGGTCCAGAGGGCC
TTCCCACTGCCCAGCCTGGAGAAGGCCTCCCCTGACCTGCTCTCTCAGTA
TCCTGGAGAGCTGGCCAGAGGCCATCACAGGCATCATCCTCAGAGCTCCT
CAGACCTGGGACTTTGTTTTTGCTGGTTCAGTGCATTTTGTGTATTTAAG
AGCAAACACTAGCCAGGCGTGGCGGCGTGTGCCTGTAGT

### CCDC93

Suitably, the agent described herein reduces an expression and/or an activity of CCDC93.

CCDC93 (Coiled-coil domain-containing protein 93) functions as a component of the CCC (COMMD/CCDC22/CCDC93) complex, which is involved in the regulation of endosomal recycling of surface proteins, including integrins, signaling receptor and channels. The nucleotide and protein sequences of CCDC93 are publicly available (e.g., Q567U6, ENST00000376300.7 and NM_019044.4). An exemplary amino acid sequence is set forth in SEQ ID NO: 5. Thus, the CCDC93 amino acid sequence may be a protein which is at least 70%, 75%, 80%, 85%, 90%, 95%, 96%, 97%, 98%, 99%, 99.5% or 100% identical to SEQ ID NO:5 or a fragment or derivative thereof. An exemplary encoding nucleotide sequence (i.e., mRNA transcript) is set forth in SEQ ID NO: 6. Thus, the CCDC93 nucleotide sequence may be a nucleic acid which is at least 70%, 75%, 80%, 85%, 90%, 95%, 96%, 97%, 98%, 99%, 99.5% or 100% identical to SEQ ID NO:6 or a fragment or derivative thereof.

CCDC93 protein
(SEQ ID NO: 5)
MGLPRGPEGQGLPEVETREDEEQNVKLTEILELLVAAGYFRARIKGLSPF
DKVVGGMTWCITTCNFDVDVDLLFQENSTIGQKIALSEKIVSVLPRMKCP
HQLEPHQIQGMDFIHIFPVVQWLVKRAIETKEEMGDYIRSYSVSQFQKTY
SLPEDDDFIKRKEKAIKTVVDLSEVYKPRRKYKRHQGAEELLDEESRIHA
TLLEYGRRYGFSRQSKMEKAEDKKTALPAGLSATEKADAHEEDELRAAEE
QRIQSLMTKMTAMANEESRLTASSVGQIVGLCSAEIKQIVSEYAEKQSEL
SAEESPEKLGTSQLHRRKVISLNKQIAQKTKHLEELRASHTSLQARYNEA
KKTLTELKTYSEKLDKEQAALEKIESKADPSILQNLRALVAMNENLKSQE
QEFKAHCREEMTRLQQEIENLKAERAPRGDEKTLSSGEPPGTLTSAMTHD
EDLDRRYNMEKEKLYKIRLLQARRNREIAILHRKIDEVPSRAELIQYQKR
FIELYRQISAVHKETKQFFTLYNTLDDKKVYLEKEISLLNSIHENFSQAM
ASPAARDQFLRQMEQIVEGIKQSRMKMEKKKQENKMRRDQLNDQYLELLE
KQRLYFKTVKEFKEEGRKNEMLLSKVKAKAS
CCDC93 nucleic acid
(SEQ ID NO: 6)
ATGCTCTGCCCGGCTGCGGCGCTTCGGGCAGGCGGCGGCGGCGGCGGCGG
CGGCGGCAGAGGGAGTTTCCGCTTTGTACTCCACCCCGGTAGCAGCTCCG
CGGCAGGGACAGCTTCCTCCGGACGCTTGGCGGGCTTCGCTCTCGCCTTA
CGACAGCCCGGTCGGATCATGGGGTTGCCCAGGGGGCCGGAGGGCCAGGG
TCTCCCGGAGGTGGAAACAAGAGAAGATGAAGAACAAAATGTCAAGTTGA
CTGAAATTCTGGAGCTCTTGGTTGCAGCTGGGTATTTCAGGGCAAGAATT
AAAGGCTTATCACCCTTTGACAAGGTAGTAGGAGGAATGACTTGGTGTAT
CACCACTTGCAACTTTGATGTAGATGTTGATTTGCTCTTTCAAGAAAACT
CTACGATAGGTCAAAAAATAGCTCTGTCAGAAAAAATTGTCTCGGTCCTG
CCAAGGATGAAATGCCCACACCAGCTGGAGCCCCACCAGATCCAGGGGAT
GGATTTTATTCACATATTTCCTGTTGTTCAGTGGCTGGTGAAACGAGCTA
TAGAAACAAAAGAAGAGATGGGTGACTATATCCGCTCCTACTCTGTATCC
CAGTTCCAGAAGACTTACAGTCTCCCTGAGGATGATGACTTCATAAAGAG
AAAAGAAAAGGCCATCAAGACAGTTGTGGACCTCTCAGAAGTGTACAAGC
CCCGTCGGAAATACAAACGCCACCAGGGAGCAGAGGAGCTACTTGATGAA
GAATCTCGAATCCATGCTACACTTTTGGAATATGGCAGGAGATATGGATT
TAGCCGCCAGAGCAAAATGGAGAAGGCTGAGGACAAGAAAACGGCACTTC
CAGCAGGGCTGTCAGCTACAGAAAAAGCTGATGCCCACGAGGAAGATGAG
CTTCGAGCAGCTGAAGAGCAGCGTATTCAGTCGCTGATGACCAAGATGAC
CGCTATGGCAAATGAGGAGAGCCGTCTCACCGCAAGCTCCGTGGGCCAGA
TTGTGGGACTCTGCTCTGCTGAGATCAAGCAGATTGTGTCCGAGTATGCA
GAGAAGCAGTCTGAGCTATCAGCTGAAGAAAGTCCAGAAAAATTAGGAAC
CTCCCAGCTACATCGCCGGAAAGTCATTTCCTTGAACAAACAGATTGCGC
AAAAGACCAAACATCTTGAAGAGCTGCGAGCAAGTCACACCAGCCTACAA
GCCAGATATAATGAAGCCAAGAAAACGCTGACAGAGCTGAAGACTTACAG
TGAGAAACTGGACAAAGAGCAAGCAGCCCTCGAGAAGATAGAATCCAAAG
CTGATCCAAGTATCCTACAGAACCTGAGAGCACTTGTAGCCATGAATGAA
AATCTGAAAAGTCAAGAACAGGAATTTAAAGCACATTGTCGAGAGGAGAT
GACACGACTACAGCAAGAAATTGAAAACCTGAAAGCTGAGAGAGCACCAC
GTGGAGATGAAAAGACCCTCTCCAGTGGAGAGCCGCCTGGTACCTTGACC
TCTGCAATGACTCATGACGAAGACCTAGACAGACGGTATAATATGGAGAA
AGAGAAACTTTACAAGATACGTTTACTACAGGCTCGAAGAAATCGAGAAA
TAGCAATTTTGCACCGCAAGATTGATGAAGTCCCTAGCCGTGCCGAGCTA
ATACAGTATCAGAAGAGATTTATTGAACTCTACCGCCAGATTTCAGCAGT
GCACAAAGAAACCAAGCAGTTCTTCACTTTATATAATACCCTGGATGATA
AAAAGGTTTATTTGGAAAAAGAGATTAGTCTGCTGAACTCAATTCATGAG
AACTTCTCACAGGCCATGGCCTCCCCTGCTGCCCGGGACCAGTTTTTACG
TCAGATGGAACAGATTGTGGAAGGAATTAAGCAAAGTAGAATGAAGATGG
AAAAGAAAAAGCAAGAGAACAAAATGAGAAGAGACCAGTTGAACGACCAG
TACTTGGAGCTGTTAGAAAAGCAGAGGCTATACTTTAAGACTGTGAAAGA
GTTCAAGGAGGAGGGCCGCAAGAACGAGATGCTGCTGTCCAAGGTGAAAG
CGAAGGCCTCCTGAACATCCCCAGCCGTGGCTGTATGTCATTGATTTTAC
TTTTAAGCACCGTATATCACCTACAAGATCATGAAATGGTTCTGAAAGCG
ACAGTAGAGAGATGCAGTTGTGATGATTTCAACAACCTGGATGTTTTCTT
TCTCCTCTTTGCTTCCATTCATCTCTGTTGGCTGCTGTTGATGGAGTCAG
ACAGTAAACACGTGGCTTGGATAACACCCATCATCCTATGAAGAATATAG
GGAGTACTTGTTCTCTGTTGATTCAACTTTTATGTCTCCAGTAACATTGC
GCTTATGAAGGTACCTGTATTTGTATGGACTCTGAATAAAGAAGAATTCA
TTTGTTTAGCAAGTATTAGTTCAGCAACCACTGAGAAATAAGCACTGAGG
AAGATTCAGAGACGTGTAAAACACAGTTCCTACTGCACAAGTACCCAGCA
GGTGGCCCAGGGAGGCAGATACAGCACACTTGACCGCAGAACTGGGCTAT
CCAAGATGTTTTTCAGTAAACAGAAGGCATTTAGCTGAAATGATCAGCCC
ATGTAGTGTTGGTCACTTGGGCCTTTCACCTGCCATGGTACCTTTTGTTC
CCAGCTCCTCCAGGTGCCAGCCAGCAGGCTTGGTGGTGACAGCAACTGGA
ACGAAAGTTCAGTGTTGTTTTAATTTTTATACGTTACTCAAGTTGATTTC
TCAGAAAATTGAAAACAGACCTTGTGCTGAGGACACGTCAATAAAAATTA
TACCTTCCCCTGCCCCAATGTTCATGTGAGTCACAGCATGCAGAGGGGCT
GTGCATCCCCCTCATTGAAGGCACATTTGATGAGGCTACGCTGCAGATGG
GTCATGGTCTGAGCCAGGAGAGAGATAGACACTGTTCCCTGCACTCAAGG
CAAGTAGGGTGTCAGGAGGAAGAGCAGTAGGGTTTGCAGAGATCCAGGGA
AACTTCCTGGAGGAGGCAGCATTTGAGCCCTATCTAAAGGGAGAATCGGA
TGTTGATAGGAAGACTGTTTTAAATGAAAATACACTATGATTAAAAACCC
AAGAGAGTGCAGAGGAGTACAAGGCATGCATGTTGGGAAAGGAGGGAATA
AGCTAGTCTCTGCAGGAGGCTTCAGAGACCCGTCTGGGAAGTAGGGCTGA
GACTAGGCTTTGGAGGGCCTTGAAGGGCAGCTGAGGGTTGATTTTAATCC
TTGGAAATGACCAGTGCAGATCAAACATGGAGCTCCTGCTGCCCTGGTCC
TCAGGAGTCTGGTAACTCAGTCATGTTCTGTAGGTTTGTGCTCCATTTTG
CATAAATCTTGCAAACATACCAGGTGGCAGGTTTGCACAGTAACACAAAA
GCTTAAACATGTCAAGCAAGTGGGGCATTCAGCAGCCACAGAAATGTGTT
CATTTTTAGTCCCTAATGGATGTGATTAAGCAGGGCTGATGTGTACAGAT
TTAAAAACACACACACGTGCTGGATTAAAAACAGCCACTGGTTTCAGTGC
CAGGGCCAGCAAGGCCTACCTGAGCCAGGCGGAAGCAGCAGCTTGCAAAT
AGAAACTGCCACCTGCGAGCGCTTCTTGGGTCATGTGCAGCTCCACCTCA
GCTGGTCCCTGAGCCGTGGTCATGTGGGGGTTTTGAAGGGCTAAGTTGAA
GCAAGTAATTGCAAAAGAACAGGTGGCACTCTGAGGGCAGGGGAAGGAGA
CAGAAGTTTGACTGCGTTTGAAATTATTTCAGTCAGGGAAACATCTGCGT
TGTCTTGTTAAGCAGCTTGTAGGCCCACCCCTGGCTAACTGAACTTTGTG
AGCAGTTGGGGCAGTTATTTGAATATTTGTCCAAAGGTGGTAGGGAGGCA
GATGACATTCTGGGGTTACTGGAGTCCACCTCTGTGCTGATGGGGAAAGT
ATGGCATGATGAATCCAAAGCAACCAGTAATTCTTTTTGAAAGTGCAGGC
ATTTATTTTGAGCTGTATTTCATAGGAGTTTCATTTGGTTTGGTTTTGGT
ATAGAGAGGTGTCCATAGATCTCTATTCCCTTTTTTTTTTTATCGACAAG
GCCTACTACGGATAAATCAGTTTTTGTTGCCTGAATACACTTTGAATTAG
ACTACAGAAGCCAGTTTGAGCTCAAATTTCACACTTGCTGCAGACAACTC
TCAGTGTGGACAGTGCCTTTCTGAGTAATTAGGATGTTACATGACTTTTT
GGTGAAGCCGTATAGGGTAAGGAGATGCCCCAAGGCCCCTGATCATTTCA
GAAGCCACCTGGAGCACCAGAAGGGGTCATCTTCCCAGGGCTATGGCTCT
GTGTGCTTAGACAGACATAGTATGGAATTGTGCTCTAAGCCCACAGGCTT
CTCCGGAGTTAAAAGGAAGCAAGAACATCCTTAAGTTACTTTTTCAAAAT
ATGCGCTTCAGGAGGAACCAGGTTTCCCCAGGAGAGAGAGGCTCCCTGCT
CAATCAGCACAGGGAGTTGCTAGCGGGCAGGCTGCCCAGTGCTGTGGAAT
CTCCGCCAGCCTCTGGGTTGGGGGCTCCCTCCCACTTCTTGCTTCTTATG
GAGTACATGGGACTGCTCTGCTTTAAGCCGCGATCAGAGCTGTTGTGAGC
AGGACAAGATTGCGTGATCACGAGGTGCGTGGGGAGATGCTAGGACATTT
GTTGATGTCTTTGACCTGTGCTCCGTCTCTGTTGGATGACTGGGTTAGCT
AACACCACTAGAGGGAGGTACAGAATGTTAAACAGTCGTCAGAGTCACAC
TGAGGGTTGTTTTAACAGAGGGCAAATTCCCAGTTTGTCAAGCATATCTC
TGTTAGAGTCCGAAGTTCATGGTTTTAATGGTTCTAGCTCGTAGCCCTTA
ATATTTTCAGGATAGCTGGGTCCTGCCAGTGTCCGCTGAGTCCATCAGCA
CAACCAGAAGGTCCAGGGTCCAGGCATGGGAAGCATTCTCTGACAATACC
GAGCCCTCGGGACCTCCTGAAACCTTGAGCAGAGGCTGGAGAAGACCTTC
AGACAGTCCTTTTACTACCTCACAAAGTGCCATGCCCGGGACCTCAGACC
TCAGGGTTGTGACGTTGTTGATGTTGTCAGGCCCCAGTACGTCTCTAGTG
ACCGAAGCAGACCTGCCTGAAAGGGCACTCCGGACAGTGAGCCCTGGCTC
TAACCTGTGCCCTCATATTTCTGCTGCCTCATATGGTCTTGTTTGCCCAC
TCTGTAGGTGACTGATCACCCTAAAAGGGAGGTAGGTATCTAGCAGTAGC
ACAGTTACTTTGAGCCTGACCACTTGTTCTGAAGTGCAGATGTTGAAACA
TGCATTGAGGAAGAACACTTTAAGGAAGCACTTTATGATTTACTCATGGC
TGGTGTTGGCCTCAAAGTAAAAGAACATCTTAATGCAGAACAGTACAATA
AAGAGCCCCATAGGATGGATGCCATCATCTGTCCCCATGGTTGGGATTGC
CAAGGAAAGTGCCTTGTTCTGTGCATCTTCAGAATCCCCCAGGCCCTCTC
TTGATCCTTTTCATGAAGTCCAGAGCATTCCTTCTATTACAGGCTGAAGA
TGCATTGTAAAAGGTAATGCTATAATAGTATTCCCTCTTCTCAATAAATG
TGCAAAACTGGAGAATCTGGTGCTGATCTGCAGCATACTTGACAACACAG
GGAATACCGTGGATGTTCCACGCCAGCCTTACATCCGTCCCCATTGCCTG
CCCATGTCCAGTGGCCCTGCGCAACAAAAGTAAGGGCTTGTCTTACCACA
CTCTCCCCAGCATCTAGCTCAAGGTAGATGACTGACTGGGAAATTCCGGT
TGGATAAAATGGCTTTTCCACATTGCAACTTTGCATCTCTATTAGGAAAA
TTTAGATTGAACCTTTCCTCTACTTGATGCTTAGTTTGAGAACAGTTTTC
ACAACTTTAGAGGGTCTCTCTCAGAGTATGTTATACTGTAACCTTGAAGT
TTGAGATGAAACAAGGTGTCAGAGGTTTTGAATCTTTCAAAGGAATTGCC
TTTTACAGATTTTTTTTTTTATTCCCTAAGAAGGGAAAAAGAGAGGGAAG
AACTACACTAATGTTAGAGATAAGGTATGTTTTGGCTCAAAATGTGTCAT
GAACCTAAAGGTAACTTGATGGGCAGAAATGTCTCAGCCATGTGAACCAA
GAAGTGGGACCACAGTGCAAGGCTGTGGGTGGCAGCACTAGATACGGTGG
TGGCAGGAGTGTGACATGTAGGCACTCCATGGGGGACAGCTGGACGGTGT
CTATCAAAAGTGCAAATGCATAAGCTCTTTGAACTAGCAATGCTACTCCT
AAGAATTGTACCTGTGTACAAGGTACTTGTGTACAAGAGTTTTTGTTGAA
ACATTGTAATAGCAAAAAGTTGGAGAGTGCCACCAATACAGGGGAATGGT
TAAATACATTATGGCCAGCTTAAACAATGGAATAGTTTGTAGAAATTTAA
AACAATGGATCCTGTGTGTGTGTACTACCATAGAATGGGCCCCAAGATGC
ATGATTAAGTGAGGAAAAAAGCCCATGGTATCCTAATGTTTGGAATGGAG
AAAGTGTGTAGATGTATGTGTGTGCATATGTTTTCACACAGACGTATACA
CGTGTATATCATATGTCCACCTGCATATGCCTAGGATGTCTCTGGGAGGA
TATCTAGCAGCCCGGCAGCCTGAATTGCCTCTGGGAAGAGGGGTGGGAAT
GACTTTTCAATGTACACCTTTTGTACCTTTTGAATTTTGTACCATGTACA
TGTATTATCTATTAAAAAATAGTTCTTAATTTTTAATTTGGTATACTTTT
CTTAATAGAACCAAAATGGAAGAAAATTCTGATTCTCTAAATCTGCTTTC
TTAAAGATAAAGCCTTTGATTCAAAAGTAAAAAAAAAAAAAAAAAA

### CHTF18

Suitably, the agent described herein reduces an expression and/or an activity of CHTF18.

CHTF18 (Chromosome transmission fidelity protein 18 homolog) is involved in sister chromatid cohesion, fidelity of chromosome transmission and DNA replication. The nucleotide and protein sequences of CHTF18 are publicly available (e.g., Q8WVB6, ENST00000262315.14 and NM_022092.2). An exemplary amino acid sequence is set forth in SEQ ID NO: 46. Thus, the CHTF18 amino acid sequence may be a protein which is at least 70%, 75%, 80%, 85%, 90%, 95%, 96%, 97%, 98%, 99%, 99.5% or 100% identical to SEQ ID NO: 46 or a fragment or derivative thereof. An exemplary encoding nucleotide sequence (i.e., mRNA transcript) is set forth in SEQ ID NO: 47. Thus, the CHTF18 nucleotide sequence may be a nucleic acid which is at least 70%, 75%, 80%, 85%, 90%, 95%, 96%, 97%, 98%, 99%, 99.5% or 100% identical to SEQ ID NO: 47 or a fragment or derivative thereof.

CHTF18 protein
(SEQ ID NO: 46)
MEDYEQELCGVEDDFHNQFAAELEVLAELEGASTPSPSGVPLFTAGRPPR
TFEEALARGDAASSPAPAASVGSSQGGARKRQVDADLQPAGSLPHAPRIK
RPRLQVVKRLNERSEEMEEPPPPDSSPTDITPPPSPEDLAELWGHGVSEA
AADVGLTRASPAARNPVLRRPPILEDYVHVTSTEGVRAYLVLRADPMAPG
VQGSLLHVPWRGGGQLDLLGVSLASLKKQVDGERRERLLQEAQKLSDTLH
SLRSGEEEAAQPLGAPEEEPTDGQDASSHCLWVDEFAPRHYTELLSDDFT
NRCLLKWLKLWDLVVFGHERPSRKPRPSVEPARVSKEATAPGKWKSHEQV
LEEMLEAGLDPSQRPKQKVALLCGPPGLGKTTLAHVIARHAGYSVVEMNA
SDDRSPEVERTRIEAATQMESVLGAGGKPNCLVIDEIDGAPVAAINVLLS
ILNRKGPQEVGPQGPAVPSGGGRRRRAEGGLLMRPIICICNDQFAPSLRQ
LKQQAFLLHFPPTLPSRLVQRLQEVSLRQGMRADPGVLAALCEKTDNDIR
ACINTLQFLYSRGQRELSVRDVQATRVGLKDQRRGLFSVWQEVFQLPRAQ
RRRVGQDPALPADTLLLGDGDAGSLTSASQRFYRVLHAAASAGEHEKVVQ
GLEDNFLRLRLRDSSLGAVCVALDWLAFDDLLAGAAHHSQSFQLLRYPPF
LPVAFHVLFASSHTPRITFPSSQQEAQNRMSQMRNLIQTLVSGIAPATRS
RATPQALLLDALCLLLDILAPKLRPVSTQLYSTREKQQLASLVGTMLAYS
LTYRQERTPDGQYIYRLEPNVEELCRFPELPARKPLTYQTKQLIAREIEV
EKMRRAEASARVENSPQVDGSPPGLEGLLGGIGEKGVHRPAPRNHEQRLE
HIMRRAAREEQPEKDFFGRVVVRSTAVPSAGDTAPEQDSVERRMGTAVGR
SEVWFRFNEGVSNAVRRSLYIRDLL
CHTF18 nucleic acid
(SEQ ID NO: 47)
GGGCAGTGCGCGACGGCGGCGGCGGCGCGGGAGGTTCGGAGCGGGAGCTC
GGGCTCGCGGACGGTATGGAGGACTACGAGCAGGAGCTGTGCGGCGTCGA
GGATGATTTCCACAACCAGTTCGCGGCCGAGCTGGAGGTGCTGGCAGAGC
TGGAAGGGGCGTCGACTCCGTCGCCCTCCGGGGTCCCCCTGTTCACCGCG
GGCCGACCCCCGCGGACGTTCGAGGAGGCCCTTGCCAGAGGGGACGCGGC
CTCCAGTCCCGCCCCAGCCGCATCTGTGGGCAGCAGCCAGGGCGGCGCCA
GGAAGAGGCAGGTGGACGCCGACCTGCAGCCGGCCGGGTCCCTGCCCCAC
GCCCCCAGGATCAAACGGCCTAGGCTGCAGGTGGTCAAGAGGCTGAACTT
CAGATCGGAGGAGATGGAGGAGCCGCCCCCTCCCGACTCCTCGCCGACGG
ACATCACCCCGCCGCCGAGCCCTGAGGACCTCGCAGAGCTTTGGGGCCAC
GGAGTCTCAGAAGCTGCTGCCGACGTGGGTCTCACACGGGCCTCACCAGC
TGCCCGCAATCCCGTCCTGAGGCGGCCCCCCATCTTGGAGGACTACGTCC
ACGTGACATCCACGGAGGGCGTCCGGGCTTATCTGGTGCTGCGTGCTGAC
CCCATGGCCCCGGGGGTGCAGGGCTCTCTCCTCCACGTCCCATGGCGAGG
CGGTGGCCAGCTGGACCTGCTGGGTGTGTCCTTAGCCTCCCTGAAGAAGC
AGGTCGACGGCGAGCGGCGGGAGCGGCTGCTTCAGGAGGCCCAGAAGCTT
TCAGACACCCTGCACAGTCTCAGGTCGGGGGAGGAGGAGGCAGCCCAGCC
CTTGGGGGCCCCTGAGGAGGAGCCGACTGACGGTCAAGACGCCTCCAGTC
ACTGCCTCTGGGTGGATGAGTTTGCACCCCGCCACTACACGGAGCTGCTC
AGTGATGACTTCACCAACCGCTGCCTGCTCAAGTGGCTGAAGTTGTGGGA
CCTGGTGGTGTTTGGCCACGAGAGGCCTTCCCGGAAGCCCAGGCCCAGTG
TTGAGCCGGCCCGGGTCAGCAAGGAGGCCACAGCCCCAGGCAAGTGGAAG
AGCCACGAACAGGTGCTGGAGGAGATGCTGGAGGCTGGGCTGGACCCGAG
CCAGCGACCGAAGCAGAAGGTGGCACTGCTCTGTGGGCCCCCGGGGCTGG
GGAAGACCACCCTGGCACACGTGATTGCGCGTCACGCGGGGTACTCTGTG
GTGGAGATGAACGCCAGTGACGACCGTAGCCCGGAGGTCTTCCGCACACG
CATCGAGGCGGCCACCCAGATGGAGTCGGTGCTGGGTGCTGGCGGGAAGC
CCAACTGCCTGGTCATCGATGAGATCGACGGGGCCCCCGTGGCCGCCATC
AACGTCCTCCTGAGCATCCTGAACCGCAAGGGGCCACAGGAGGTGGGGCC
ACAGGGCCCGGCTGTGCCTTCGGGAGGCGGCCGACGGCGCCGGGCAGAGG
GGGGGCTCCTCATGAGGCCCATTATCTGCATTTGCAATGACCAGTTCGCA
CCGTCCCTGCGGCAGCTGAAGCAGCAGGCCTTCCTGCTCCACTTCCCGCC
GACTCTGCCCTCGAGGCTGGTGCAGCGGCTCCAGGAGGTCTCCCTGCGGC
AGGGCATGAGGGCCGACCCAGGGGTGCTGGCCGCCCTCTGTGAGAAAACT
GACAATGACATCCGGGCCTGCATCAACACCCTGCAGTTCCTGTACAGCCG
GGGCCAGCGGGAGCTGAGCGTGCGGGACGTGCAGGCCACACGCGTGGGCC
TCAAGGACCAGCGCAGAGGGCTCTTCTCGGTGTGGCAGGAGGTCTTCCAG
CTGCCTCGAGCCCAGAGGCGCCGTGTGGGCCAGGACCCCGCCCTGCCTGC
TGACACACTCCTGCTGGGTGACGGGGACGCGGGCTCCCTCACCTCCGCCT
CACAGCGATTCTACCGTGTCCTGCATGCCGCTGCCTCTGCGGGCGAGCAC
GAGAAGGTGGTCCAGGGCTTGTTTGACAACTTCCTGCGTCTGCGGCTGCG
AGACTCCAGCCTGGGTGCTGTGTGTGTGGCCCTCGACTGGCTGGCCTTCG
ATGACCTGCTGGCGGGGGCTGCTCATCACAGCCAGAGCTTCCAGCTGCTG
CGCTACCCACCCTTCCTGCCCGTGGCCTTCCATGTGCTGTTTGCTTCCAG
CCACACACCCAGGATCACCTTCCCCAGCAGCCAGCAGGAGGCCCAGAACC
GGATGAGCCAGATGAGGAACCTGATCCAGACGCTGGTGTCCGGCATCGCG
CCAGCCACGCGCAGCCGGGCCACGCCCCAGGCCCTGCTCCTCGATGCCCT
CTGCCTGCTCCTGGACATTCTTGCACCCAAGCTCCGCCCCGTGAGCACAC
AGCTGTACAGCACCCGTGAAAAGCAACAGCTGGCCAGCCTGGTGGGCACG
ATGCTCGCTTACAGCCTGACCTACCGCCAGGAGCGCACGCCCGATGGCCA
GTACATCTACAGGCTGGAGCCGAACGTGGAGGAACTCTGCCGCTTCCCTG
AGCTGCCTGCCCGCAAGCCCCTCACCTACCAGACGAAGCAGCTCATCGCC
CGCGAGATCGAGGTGGAGAAGATGCGGCGGGCGGAGGCTTCTGCCCGGGT
AGAGAACAGCCCCCAGGTGGATGGGAGCCCCCCAGGGCTCGAGGGTCTGC
TGGGGGGCATTGGGGAGAAAGGGGTGCACCGACCTGCCCCACGCAACCAT
GAGCAGCGGCTGGAGCACATCATGAGGCGAGCGGCCCGGGAGGAACAGCC
TGAGAAGGACTTCTTTGGACGTGTGGTCGTCAGGAGCACAGCAGTCCCGA
GTGCAGGGGACACGGCCCCGGAGCAGGACTCAGTGGAGCGGCGCATGGGC
ACAGCGGTGGGCAGGAGCGAGGTCTGGTTCCGCTTCAACGAGGGTGTCTC
CAACGCCGTGCGGCGCAGCCTGTACATCAGGGACTTGCTCTAGTTCTCTG
AGCCGCGGACATGCCCTCGCATTGCTTCCCGCAGAGTGCAGAGACAGGAA
GCTGGAGATGTCTTTATAAAGTCACACCTTTACAGACTGTAA

### ETAA1

Suitably, the agent described herein reduces an expression and/or an activity of ETAA1.

ETAA1 (Ewing's tumor-associated antigen 1) functions as a replication stress response protein that accumulates at DNA damage sites and promotes replication fork progression and integrity. The nucleotide and protein sequences of ETAA1 are publicly available (e.g., Q9NY74, ENST00000272342.6 and NM_019002.3). An exemplary amino acid sequence is set forth in SEQ ID NO: 7. Thus, the ETAA1 amino acid sequence may be a protein which is at least 70%, 75%, 80%, 85%, 90%, 95%, 96%, 97%, 98%, 99%, 99.5% or 100% identical to SEQ ID NO:7 or a fragment or derivative thereof. An exemplary encoding nucleotide sequence (i.e., mRNA transcript) is set forth in SEQ ID NO: 8. Thus, the ETAA1 nucleotide sequence may be a nucleic acid which is at least 70%, 75%, 80%, 85%, 90%, 95%, 96%, 97%, 98%, 99%, 99.5% or 100% identical to SEQ ID NO:8 or a fragment or derivative thereof.

ETAA1 protein
(SEQ ID NO: 7)
MSRRRKHDDSPSPKKTPHKTVAAEECGSVVEPGRRRLRSARGSWPCGARE
GPPGPVRQREQPPTAALCSKSNPEERYETPKRALKMDSLSSSFSSPNDPD
GQNDIFWDQNSPLTKQLGKGRKKQIYTTDSDEISHIVNRIAPQDEKPTTN
SMLDMWIGETAIPCTPSVAKGKSRAKISCTKLKTQSQEEELMKLAKQFDK
NMEELDVIQEQNKRNYDFTQMISETEILSNYKDNIQMWSLHNIVPEIDNA
TKKPIKGNTKISVANNQNSSQKPFDQIAEAAFNAIFDGSTQKCSGQLSQE
LPEAFWSTSNTTFVKTNALKEEKIITNETLVIEKLSNKTPRSLSSQVDTP
IMTKSCVTSCTKEPETSNKYIDAFTTSDFEDDWENLLGSEPFAMQNIDMP
ELFPSKTAHVTDQKEICTENSKTVKNTSRANTSPDARLGDSKVLQDLSSK
TYDRELIDAEYRFSPNSNKSNKLSTGNKMKFENSSNKIVIQDEIQNCIVT
SNLTKIKEDILTNSTEASERKSALNTRYSNEQKNKCILNQSIKAPVNTDL
FGSANLGSKTSVSNPNQTSASKVGSFFDDWNDPSFANEIIKACHQLDNTW
EADDVDDDLLYQACDDIERLTQQQDIRKDSKTSESICEINNNSEHGAKLT
QQQDIRKDSKTSESICEINNNSEHGAKNMFAISKQGSNLVQSKHLNPGSI
SVQTSLINSSQIDKPMKMEKGEMYGNSPRFLGATNLTMYSKISNCQINNL
HVSYTNTDVPIQVNSSKLVLPGSSSLNVTSDHMNTEITTYKKKLSTNQPC
HKTVTDEAQSNLNTTVGFSKFTFTRMKNSQILSQFNQNCITGSMSDTKIT
QGVEKKKGVNPLLEEAVGQQSLVKLSESLKQSSKEEEEKNRKCSPEEIQR
KRQEALVRRMAKARASSVNAAPTSFL
ETAA1 nucleic acid
(SEQ ID NO: 8)
CGGCCGCCTCTTGCGCGCGCCCCACCGACCAAAATGGCGGCTGCCGTTGG
TGCGGGGTGCGGTTTGTAGTGCTGTTGCCCTACTCATCCCTTTGCAAAAT
GTGAAAGAAGAAGCGGCTGGTGGAGGCGGGCCATAGGCAATGAGTCGGCG
AAGGAAACATGATGACAGCCCTAGCCCGAAGAAAACGCCGCACAAAACAG
TGGCGGCGGAGGAATGCGGCTCGGTGGTCGAGCCAGGGAGGAGGCGGCTG
AGATCGGCCCGCGGTTCGTGGCCCTGCGGGGCTAGAGAGGGGCCTCCCGG
GCCAGTGCGGCAGCGAGAGCAGCCTCCGACCGCCGCCCTGTGCAGTAAAA
GTAACCCCGAGGAAAGGTATGAAACACCAAAGAGAGCGCTGAAAATGGAC
TCACTGTCATCTTCCTTCAGTTCTCCTAATGATCCAGATGGACAGAATGA
TATCTTTTGGGATCAGAATTCTCCATTGACAAAGCAGTTAGGTAAAGGAA
GAAAAAAACAGATTTACACCACAGATAGTGATGAGATTTCACATATTGTT
AATCGTATTGCTCCTCAGGATGAAAAACCAACAACAAATTCTATGCTGGA
CATGTGGATTGGTGAAACTGCTATTCCTTGTACTCCCAGTGTAGCAAAAG
GAAAATCAAGAGCAAAAATCAGCTGCACAAAGTTAAAAACACAAAGTCAA
GAAGAAGAACTTATGAAACTGGCTAAACAATTTGATAAAAATATGGAAGA
GCTAGATGTGATTCAAGAGCAAAACAAGAGGAATTATGATTTTACCCAGA
TGATTTCAGAAACAGAGATTTTAAGTAATTATAAAGATAATATACAGATG
TGGTCATTACATAATATAGTTCCCGAAATAGATAATGCTACAAAAAAGCC
AATCAAAGGAAACACCAAGATATCTGTGGCAAATAATCAAAATAGCAGTC
AGAAGCCATTTGACCAAATTGCTGAAGCAGCCTTTAATGCTATTTTTGAT
GGTTCTACTCAGAAATGTAGCGGACAGTTAAGCCAAGAACTGCCAGAGGC
TTTTTGGAGCACCAGTAATACTACCTTTGTAAAGACAAATGCTTTGAAAG
AGGAGAAAATCATTACTAATGAAACTCTGGTCATTGAAAAACTGTCAAAT
AAAACCCCACGATCACTTTCTTCTCAAGTAGATACACCCATAATGACAAA
ATCATGTGTGACTTCCTGTACTAAGGAGCCAGAAACTTCTAATAAGTACA
TTGATGCATTTACTACAAGTGATTTTGAGGATGATTGGGAAAACTTACTA
GGTAGTGAACCTTTTGCTATGCAAAATATCGACATGCCTGAACTCTTTCC
TTCTAAAACAGCCCATGTTACTGATCAAAAGGAAATTTGTACCTTTAATA
GTAAAACTGTTAAAAATACGTCAAGAGCAAATACAAGTCCAGATGCCAGG
TTAGGAGATTCAAAAGTATTACAAGATCTTTCTTCAAAGACATATGACAG
AGAATTAATAGATGCAGAATATAGATTTTCACCAAATTCAAATAAATCAA
ACAAATTATCCACTGGAAATAAAATGAAATTTGAGAACTCTTCCAATAAA
ATTGTTATTCAAGACGAAATTCAAAATTGTATAGTTACATCTAATCTGAC
AAAAATAAAGGAAGATATTCTTACTAACTCTACTGAAGCTTCTGAAAGGA
AGTCAGCTTTGAACACAAGGTATTCTAATGAACAGAAAAATAAGTGCATT
TTAAATCAGTCTATTAAAGCCCCTGTTAATACTGATCTTTTTGGCTCTGC
AAATCTAGGCAGTAAAACCAGTGTTAGTAACCCAAATCAGACTAGTGCAT
CAAAAGTAGGTTCTTTCTTTGATGATTGGAATGATCCCTCATTTGCCAAT
GAAATTATTAAAGCATGTCATCAATTAGATAATACCTGGGAAGCAGATGA
TGTAGATGATGATTTGTTGTACCAAGCATGTGATGATATTGAAAGACTAA
CTCAGCAACAAGACATTAGAAAGGACAGTAAGACATCAGAAAGTATATGT
GAGATCAATAATAATTCCGAACATGGAGCCAAACTAACTCAGCAACAAGA
CATTAGAAAGGACAGTAAGACATCAGAAAGTATATGTGAGATCAATAATA
ATTCCGAACATGGAGCCAAAAACATGTTTGCTATATCTAAACAAGGAAGT
AATTTGGTACAATCAAAGCATTTGAATCCAGGCAGCATTTCAGTGCAGAC
ATCTTTGACAAATAGCTCACAAATAGATAAGCCAATGAAGATGGAGAAAG
GGGAAATGTATGGAAATTCTCCAAGATTTTTAGGTGCCACAAATTTGACT
ATGTATTCTAAGATCTCAAACTGTCAGATAAATAATCTGCATGTGTCTTA
TACTAACACTGATGTTCCAATACAAGTGAATAGTTCCAAATTGGTTCTTC
CAGGAAGTTCAAGTTTGAATGTAACTTCAGATCATATGAATACAGAAATT
ACTACTTATAAGAAGAAATTGAGTACTAATCAGCCATGCCATAAGACTGT
AACAGATGAAGCTCAGAGCAACCTTAACACAACAGTTGGATTTTCAAAGT
TTACATTTACAAGGATGAAAAATTCTCAGATTCTTTCTCAGTTTAATCAA
AATTGTATAACTGGAAGTATGTCTGATACCAAAATTACACAGGGTGTGGA
GAAAAAGAAAGGTGTCAACCCATTACTGGAGGAAGCTGTTGGACAGCAAT
CTTTGGTGAAACTTTCTGAATCTTTGAAACAATCTTCAAAAGAGGAAGAA
GAGAAAAATAGAAAGTGTTCTCCTGAAGAAATTCAGAGAAAAAGACAAGA
AGCACTGGTTCGGAGAATGGCTAAAGCACGAGCCTCATCTGTAAATGCAG
CTCCCACTTCATTTCTTTAATGAAATATTAGTTGGAAGACTTCACGAAGA
CTGCTGATAACTATCTGTGATTGATAGGAAATTTTTTTTCTTGATTTCTC
TGTGAGAAATGTAATGCTGACTTTTATAAAGCCTGGACTTCTACTTTATT
TAATAAATCAATGTTTGCAATGGTAAATGAAACATTTCCTTGGACATGTA
TTTGAAAGTCATTAAATACAAAAGTTTTGGAAATTCAGGAAAGTTAGCAA
TTATGTACGGATATTATACAGAGGAAAGTAGTTATATTTTTAAATGCTAT
TATTGCAGAGGATCATCAAAAAAGAGGTAATCTACGTTATTTCCTATTCT
AATGTCTTTTCCTAATAAAAAACTTCAACTTTCAAAAAAAAAAAAAAA

### ZMAT1

Suitably, the agent described herein reduces an expression and/or an activity of ZMAT1.

ZMAT1 (Zinc finger matrin-type protein 1) contains Cys2-His2 (C2H2)-type zinc fingers, which are similar to those found in the nuclear matrix protein matrin 3. The nucleotide and protein sequences of ZMAT1 are publicly available (e.g., Q5H9K5, ENST00000651725.2 and NM_001282400.2). An exemplary amino acid sequence is set forth in SEQ ID NO: 9. Thus, the ZMAT1 amino acid sequence may be a protein which is at least 70%, 75%, 80%, 85%, 90%, 95%, 96%, 97%, 98%, 99%, 99.5% or 100% identical to SEQ ID NO: 9 or a fragment or derivative thereof. An exemplary encoding nucleotide sequence (i.e., mRNA transcript) is set forth in SEQ ID NO: 10. Thus, the ZMAT1 nucleotide sequence may be a nucleic acid which is at least 70%, 75%, 80%, 85%, 90%, 95%, 96%, 97%, 98%, 99%, 99.5% or 100% identical to SEQ ID NO: 10 or a fragment or derivative thereof.

ZMAT1 protein
(SEQ ID NO: 9)
MESCSVTRLECSGAISAHCSLHLPGSSDSPASASQIAGTTDAIWNEQEKA
ELFTDKFCQVCGVMLQFESQRISHYEGEKHAQNVSFYFQMHGEQNEVPGK
KMKMHVENFQVHRYEGVDKNKFCDLCNMMESSPLIAQSHYVGKVHAKKLK
QLMEEHDQASPSGFQPEMAFSMRTYVCHICSIAFTSLDMFRSHMQGSEHQ
IKESIVINLVKNSRKTQDSYQNECADYINVQKARGLEAKTCFRKMEESSL
ETRRYREVVDSRPRHRMFEQRLPFETFRTYAAPYNISQAMEKQLPHSKKT
YDSFQDELEDYIKVQKARGLDPKTCFRKMRENSVDTHGYREMVDSGPRSR
MCEQRFSHEASQTYQRPYHISPVESQLPQWLPTHSKRTYDSFQDELEDYI
KVQKARGLEPKTCFRKIGDSSVETHRNREMVDVRPRHRMLEQKLPCETFQ
TYSGPYSISQVVENQLPHCLPAHDSKQRLDSISYCQLTRDCFPEKPVPLS
LNQQENNSGSYSVESEVYKHLSSENNTADHQAGHKQKHQKRKRHLEEGKE
RPEKEQSKHKRKKSYEDTDLDKDKSIRQRKREEDRVKVSSGKLKHRKKKK
SHDVPSEKEERKHRKEKKKSVEERTEEEMLWDESILGF
ZMAT1 nucleic acid
(SEQ ID NO: 10)
GTCCGCGCGCCTCGCTGCGATGGCGGCGGCGCCGAGCACAGTCACCCCGC
TGGCGGCGGAGTCTTCCCCTCAGGAAGCTACCGTCTCTGCCGCCTCCTCC
TCCTCCTACACCGCCTGCGCGGCAGCGGCGGCGGCGGCGGCGGCGGCGGC
AGTAATTGTTCCTGCCTCCTCCGCCACCTCCGCCCCTGCCTGCCCGCCTG
CCGGTGGCTGTGGCGACGGCGGCGGCGGCGGCTTTGGCGGCTCCACTATG
GCGGCGGCGGGGAGGGGGGGCAGTAGTTTTAAAGTAGACACCCGCCCTTG
CCTCAGAGAAGACGCCATTTGGAATGAACAGGAAAAGGCTGAACTTTTTA
CAGATAAGTTTTGTCAAGTATGTGGAGTGATGCTACAGTTTGAATCACAA
AGAATTTCACATTATGAGGGTGAAAAACATGCTCAAAATGTTAGTTTTTA
TTTTCAAATGCATGGGGAACAAAATGAAGTGCCTGGTAAGAAAATGAAGA
TGCATGTTGAGAATTTTCAGGTGCATAGGTATGAAGGAGTGGACAAAAAC
AAATTTTGTGATCTCTGCAACATGATGTTTAGCTCTCCACTTATTGCTCA
GTCTCACTATGTGGGAAAGGTCCATGCTAAAAAACTGAAGCAATTAATGG
AGGAACATGATCAGGCATCTCCATCAGGATTTCAACCAGAGATGGCAGGA
GTGCCTCTTACTACTTCTGCAGAGTCAACATTCCTGAAGCCCCTTCCTGT
CAAGCCTCCTACAGGCACCTTCTCACTACTAGTGTATTTTATTCCCCTTT
CATATTCTCTCTTCCACCCACTTCTTTTCCTCCACTCACTATTATTTTTC
AGTTCTTTCTTTACCCAGCCTTTCTCCTTCATGTGAAAATGTTCATTCTT
CTCTCCTCCTTGAAGCTCTATTCTTTGTCATATGTAACTCTTTATGCTCT
GGTTCTTCTCTATTTCTGTTTCTTTTTTGTTTTCTCTATGGGCTCTTTTT
TTATTCATTACCTAAATGTAGGTATTCCCATGGTTTCATCCTTGACCTTA
CTCTACAAACACGATTTCTATTTCCCTGATTTCATTTCTCATCTGTATGC
TTATGACCTTTCTGTCAAGTATTAGATTCACATATCCAAATGGCAGCTAC
TGCTTCACCTGGATGCCTCATAGCCATTTCAAATCCACCTAGTCATTCAA
AGTAGAAACCCACAGCTCAGATCTTATTAGCACAGGAATGGCTACCATAA
CGATATGGAACTAAAAATTTTGCTTTCAGCTTTTATCCTGAGAGTGGGGC
TCACTGAACCTCTCTCCTTTCTAAAATCTGTGGAAATTACTTTTGGTAAC
TAAGGCACTGCAGTGATCTCAGCATTTAGTATGAGAACCTATGTTTGCCA
TATTTGTAGTATTGCTTTTACATCTTTAGATATGTTCCGGTCCCACATGC
AAGGAAGTGAACATCAAATTAAAGAATCCATTGTTATCAATCTAGTGAAG
AATTCAAGGAAGACACAAGACTCTTACCAAAATGAGTGTGCAGATTACAT
CAATGTGCAGAAAGCCAGAGGACTAGAGGCCAAGACTTGTTTCAGAAAGA
TGGAAGAGAGTTCTTTGGAAACCCGTAGATACAGAGAAGTGGTCGATTCC
AGACCCAGACATAGAATGTTTGAACAAAGACTCCCATTTGAGACTTTCCG
GACATACGCAGCACCATACAATATTTCACAAGCAATGGAAAAGCAGTTAC
CTCATTCAAAGAAGACATATGACTCTTTCCAAGATGAACTTGAAGATTAC
ATCAAAGTACAGAAAGCCAGAGGACTAGATCCAAAGACTTGTTTCAGAAA
GATGAGAGAGAACTCTGTGGATACTCATGGGTACAGAGAAATGGTTGATT
CTGGACCCAGATCAAGAATGTGTGAGCAAAGATTTTCACATGAGGCTTCC
CAGACCTACCAACGACCATACCATATTTCACCAGTGGAAAGCCAGTTACC
TCAGTGGCTACCAACCCATTCAAAGAGGACATATGATTCTTTCCAAGATG
AACTTGAAGATTACATAAAAGTGCAGAAAGCCAGAGGACTAGAGCCAAAA
ACTTGTTTCAGAAAGATAGGAGATAGCTCTGTAGAAACACACAGGAACAG
AGAAATGGTTGATGTCAGACCCAGACATAGAATGTTGGAGCAAAAGCTCC
CATGTGAGACTTTCCAGACCTATTCAGGACCATATAGTATTTCACAAGTA
GTGGAAAACCAGTTACCTCATTGCTTACCAGCTCATGATAGCAAACAGAG
ACTAGATTCTATTAGCTACTGTCAACTCACCAGAGACTGTTTCCCAGAAA
AACCAGTACCCTTGAGCCTTAATCAGCAAGAAAATAACTCTGGCTCATAC
AGTGTAGAATCTGAAGTTTACAAGCACCTCTCTTCAGAAAACAATACTGC
TGACCATCAAGCAGGTCATAAACGGAAACATCAGAAGAGAAAACGACACC
TAGAAGAAGGCAAAGAAAGGCCAGAGAAAGAGCAGTCCAAGCATAAAAGG
AAAAAGAGTTATGAAGATACAGATTTAGACAAAGACAAGAGCATCAGACA
AAGGAAAAGAGAGGAGGATAGAGTCAAGGTCAGTTCAGGAAAGCTTAAGC
ATCGAAAAAAGAAAAAAAGCCATGATGTACCCTCCGAGAAAGAAGAACGT
AAGCACAGGAAAGAGAAAAAGAAATCTGTTGAAGAAAGGACAGAAGAGGA
AATGCTTTGGGATGAGTCTATTCTTGGATTTTGAATGTTTAGTTTTGTTT
ACCCAAGGTTGAATTGAAAAAAAAAAACAGTCAATATGGATTTAGAAAAA
GGAACACCTGATGAAGAAAAGGAGAGGTAGATACAGTCAGTGTCACTTCA
GGACACTTAGGTTTTTTTTGTATAAAAATTTAAATTGAATTAAAAGAAGG
AAAAAAAAAGCCCAAACTTAACCTCTGAGAAAAGAACATAAGAACTCAAG
GAGAACATAAGAGAAAAGGAAACCTGTTACAGAAAAGACAAGAATCTGTG
TTTTGGAATGAGTCTATTCTTGGGTATTGAACTTTTAGTTTTGTTTGCCC
AAGGATTAATTGAGGAAATCAGCTAAGAAAATGGACTTTAGACAAAAGCA
AGAGGATCAGATGAAGAAAAGGAGAGGTAGATACAGTCAGTGTCACTTCA
GGAAAGCTATTTAAAAAAACTTGAAATTTAATTGAAAGAAGAAACAACAA
CAAAAAAGCCTAAACCTAGCCTCTGAACAACACTAACATGAGAACACAAG
AACTTAAGAGAAAAAGAAACCTACTCAAGAAAAGACAGAAGAGACAGTGA
TTTGGGATGAGTCTACTCTAGGATTTTCAACTTTTTAGTTTTGTTCCTTC
AAAGTTGAAGGAAAAAAAGTTTGGTTTTATAAAATTCATGTTATTGTAAT
TTTTCTAGGTGGATGGCTACTTTAATCTCTAAAAAAGCCAAGTGAAGTAA
AAGTATTCAGTATGCCTTTTCCTCAAGTTACTTTCCTTCATTTTCTTAAA
AAAGAAAAAAAATTATTAAATGTTTCTCACATATCTCACATATAATGTAA
TTTCCCTAAATGAAGTTGTCTCTACTTCTGCTCATCAAATTGCTGTGATA
GTGAATTATTTATTCATGGGAGATAATTTATTTTAAAGGACAGAATTACC
AAGCGTTACAAAATCAGTTCTTTCCTTGGTTTTGTGTTAGTGTTGGTGGT
ATTTTATTGTTGTTTTTCTGTGTTTATGTGTCTCAGCTTTCTCCAAGGAA
TATGTATGAAATAACTTAAACTGATTTTTTCTTTGTTAAATCTAATTTGC
AGTGTATTTTTGCATTTTCTAGTTCTGAAAGTGGAAAATGAAACAGTCTA
TAATAAACTTAGATGATATATAGTTTTAAAACGGTCTCAAAAAGTACTGA
TATAAGGTCAGTCTATATTCTGGAAATGTTTATATTAAAGTGTTTTAATT
TCTA

### SIVA1

Suitably, the agent described herein reduces an expression and/or an activity of SIVA1.

SIVA1 (Apoptosis regulatory protein Siva) is an E3 ubiquitin ligase that regulates cell cycle progression, cell proliferation and apoptosis. The nucleotide and protein sequences of SIVA1 are publicly available (e.g., 015304, ENST00000329967.11 and NM_006427.4). An exemplary amino acid sequence is set forth in SEQ ID NO: 11. Thus, the SIVA1 amino acid sequence may be a protein which is at least 70%, 75%, 80%, 85%, 90%, 95%, 96%, 97%, 98%, 99%, 99.5% or 100% identical to SEQ ID NO: 11 or a fragment or derivative thereof. An exemplary encoding nucleotide sequence (i.e., mRNA transcript) is set forth in SEQ ID NO: 12. Thus, the SIVA1 nucleotide sequence may be a nucleic acid which is at least 70%, 75%, 80%, 85%, 90%, 95%, 96%, 97%, 98%, 99%, 99.5% or 100% identical to SEQ ID NO: 12 or a fragment or derivative thereof.

SIVA1 protein
(SEQ ID NO: 11)
MPKRSCPFADVAPLQLKVRVSQRELSRGVCAERYSQEVFEKTKRLLFLGA
QAYLDHVWDEGCAVVHLPESPKPGPTGAPRAARGQMLIGPDGRLIRSLGQ
ASEADPSGVASIACSSCVRAVDGKAVCGQCERALCGQCVRTCWGCGSVAC
TLCGLVDCSDMYEKVLCTSCAMFET
SIVA1 nucleic acid
(SEQ ID NO: 12)
GTCGTTGGTAAGGGGCTGGCGGCCGGGGAGCTGCGTAGCTCCCGGCCCCG
CGGCCATGCCCAAGCGGAGCTGCCCCTTCGCGGACGTGGCCCCGCTACAG
CTCAAGGTCCGCGTGAGCCAGAGGGAGTTGAGCCGCGGCGTGTGCGCCGA
GCGCTACTCGCAGGAGGTCTTCGAGAAGACCAAGCGACTCCTGTTCCTCG
GGGCCCAGGCCTACCTGGACCACGTGTGGGATGAAGGCTGTGCCGTCGTT
CACCTGCCAGAGTCCCCAAAGCCTGGCCCTACAGGGGCCCCGAGGGCTGC
ACGTGGGCAGATGCTGATTGGACCAGACGGCCGCCTGATCAGGAGCCTTG
GGCAGGCCTCCGAAGCTGACCCATCTGGGGTAGCGTCCATTGCCTGTTCC
TCATGCGTGCGAGCCGTGGATGGGAAGGCGGTCTGCGGTCAGTGTGAGCG
AGCCCTGTGCGGGCAGTGTGTGCGCACCTGCTGGGGCTGCGGCTCCGTGG
CCTGTACCCTGTGTGGCCTCGTGGACTGCAGTGACATGTACGAGAAAGTG
CTGTGCACCAGCTGTGCCATGTTCGAGACCTGAGGCTGGCTCAAGCCGGC
TGCCTTCACCGGGAGCCACGCCGTGCATGGCAGCCTTCCCTGGACGAGCG
CTCGGTGTTCACACTGAACTGTGGGGTCGACGGGAGGGGTGCCTTTTACA
TGTTCTATTTTGTATCCTAATGACAGAATGAATAAACCTCTTTATATTTG
CA

### FZR1

Suitably, the agent described herein reduces an expression and/or an activity of FZR1.

FZR1 (Fizzy-related protein homolog) is a substrate-specific adapter for the anaphase promoting complex/cyclosome (APC/C) E3 ubiquitin-protein ligase complex. The nucleotide and protein sequences of FZR1 are publicly available (e.g., Q9UM11, ENST00000441788.7 and NM_001136197.1). An exemplary amino acid sequence is set forth in SEQ ID NO: 13. Thus, the FZR1 amino acid sequence may be a protein which is at least 70%, 75%, 80%, 85%, 90%, 95%, 96%, 97%, 98%, 99%, 99.5% or 100% identical to SEQ ID NO: 13 or a fragment or derivative thereof. An exemplary encoding nucleotide sequence (i.e., mRNA transcript) is set forth in SEQ ID NO: 14. Thus, the FZR1 nucleotide sequence may be a nucleic acid which is at least 70%, 75%, 80%, 85%, 90%, 95%, 96%, 97%, 98%, 99%, 99.5% or 100% identical to SEQ ID NO: 14 or a fragment or derivative thereof.

FZR1 protein
(SEQ ID NO: 13)
MDQDYERRLLRQIVIQNENTMPRVTEMRRTLTPASSPVSSPSKHGDRFIP
SRAGANWSVNFHRINENEKSPSQNRKAKDATSDNGKDGLAYSALLKNELL
GAGIEKVQDPQTEDRRLQPSTPEKKGLFTYSLSTKRSSPDDGNDVSPYSL
SPVSNKSQKLLRSPRKPTRKISKIPFKVLDAPELQDDFYLNLVDWSSLNV
LSVGLGTCVYLWSACTSQVTRLCDLSVEGDSVTSVGWSERGNLVAVGTHK
GFVQIWDAAAGKKLSMLEGHTARVGALAWNAEQLSSGSRDRMILQRDIRT
PPLQSERRLQGHRQEVCGLKWSTDHQLLASGGNDNKLLVWNHSSLSPVQQ
YTEHLAAVKAIAWSPHQHGLLASGGGTADRCIRFWNTLTGQPLQCIDTGS
QVCNLAWSKHANELVSTHGYSQNQILVWKYPSLTQVAKLTGHSYRVLYLA
MSPDGEAIVTGAGDETLRFWNVESKTRSTKVKWESVSVLNLFTRIR
FZR1 nucleic acid
(SEQ ID NO: 14)
GCTAACCTTGCCGCGGGCCGAGCCCTGCCTCGCCATGGACCAGGACTATG
AGCGGCGCCTGCTTCGCCAGATCGTCATCCAGAATGAGAACACGATGCCA
CGCGTCACAGAGATGCGGCGGACCCTGACGCCTGCCAGCTCCCCAGTGTC
CTCGCCCAGCAAGCACGGAGACCGCTTCATCCCCTCCAGAGCCGGAGCCA
ACTGGAGCGTGAACTTCCACAGGATTAACGAGAATGAGAAGTCTCCCAGT
CAGAACCGGAAAGCCAAGGACGCCACCTCAGACAACGGCAAAGACGGCCT
GGCCTACTCTGCCCTGCTCAAGAATGAGCTGCTGGGTGCCGGCATCGAGA
AGGTGCAGGACCCGCAGACTGAGGACCGCAGGCTGCAGCCCTCCACGCCT
GAGAAGAAGGGTCTGTTCACGGTGACGCGGCTCTGTGACCTCTCAGTGGA
AGGGGACTCAGTGACCTCCGTGGGCTGGTCTGAGCGGGGGAACCTGGTGG
CGGTGGGCACACACAAGGGCTTCGTGCAGATCTGGGACGCAGCCGCAGGG
AAGAAGCTGTCCATGTTGGAGGGCCACACGGCACGCGTCGGGGCGCTGGC
CTGGAATGCTGAGCAGCTGTCGTCCGGGAGCCGCGACCGCATGATCCTGC
AGAGGGACATCCGCACCCCGCCACTGCAGTCGGAGCGGCGGCTGCAGGGC
CACCGGCAGGAGGTGTGCGGGCTCAAGTGGTCCACAGACCACCAGCTCCT
CGCCTCGGGGGGCAACGACAACAAGCTGCTGGTCTGGAATCACTCGAGCC
TGAGCCCCGTGCAGCAGTACACGGAGCACCTGGCGGCCGTGAAGGCCATC
GCCTGGTCCCCACATCAGCACGGGCTGCTGGCCTCGGGGGGCGGCACAGC
TGACCGCTGTATCCGCTTCTGGAACACGCTGACAGGACAACCACTGCAGT
GTATCGACACGGGCTCCCAAGTGTGCAATCTGGCCTGGTCCAAGCACGCC
AACGAGCTGGTGAGCACGCACGGCTACTCACAGAACCAGATCCTTGTCTG
GAAGTACCCCTCCCTGACCCAGGTGGCCAAGCTGACCGGGCACTCCTACC
GCGTGCTGTACCTGGCAATGTCCCCTGATGGGGAGGCCATCGTCACTGGT
GCTGGAGACGAGACCCTGAGGTTCTGGAACGTCTTTAGCAAAACCCGTTC
GACAAAGGAGTCTGTGTCTGTGCTCAACCTCTTCACCAGGATCCGGTAAA
CCTGCCGGGCAGGACCGTGCCACACCAGCTGTCCAGAGTCGGAGGACCCC
AGCTCCTCAGCTTGCATGGACTCTGCCTTCCCAGCGCTTGTCCCCCGAGG
AAGGCGGCTGGGCGGGGGGGGAGCTGGGCCTGGAGGATCCTGGAGTCTCA
TTAAATGCCTGATTGTGAACCATGTCCACCAGTATCTGGGGTGGGCACGT
GGTCGGGGACCCTCAGCAGCAGGGGCTCTGTCTCCCTTCCCAAAGGGCGA
GAACCACATTGGACGGTCCCGGCTCAGACCGTCTGTACTCAGAGCGACGG
ATGCCCCCTGGGACCCTCACTGCCTCCGTCTGTTCATCACCTGCCCACCG
GAGCCGCATGCTCTTCCTGGAACTGCCCACGTCTGCACAGAACAGACCAC
CAGACGCCAGGGCTGATTGGTGGGGGCCTGAGACCCCGGTTGCCCATTCA
TGGCTGCACCCCACCATGTCAAACCCAAGACCAGCCCCAAGGCCAGACCA
AGGCATGTAGGCCTGGGCAGGTGGCTCGGGGCCACTGGCGGAGCCAGCCT
GTGGATCCAAGAGACAGTCCCCACCTGGGCTTCACGGCATCCTTGCAGCC
ACCTCTGCTGTCACTGCTCGAAGCAGCAGTCTCTCTGGAAGCATCTGTGT
CATGGCCATCGCCCGGCGGTCAGTGGGCTTCAGATGGGCCTGTGCATCCT
GGCCAAGCGTCACCCTCACACTGGAGGAGGATGTCTGCTCTGGACTTATC
ACCCCAGGAGAACTGAACCCGGACCTGCTCACTGCCCTGGCTGGAGAGGA
GCACAACAGATGCCACGTCTTCGTGCATTCGCCAACACGTGCCCTCACAG
GGCCAGCGTCCTCCTTCCCTGCGCAAGACTTGCGTCCCCCATGCCTGCTG
GGTGGCTGGGTCCTGTGGAGGCCAGCAGCGGTGTGGCCCCCGCCCCCAGG
CTGCCTGTGTCTTCACCTGTCCTGTCCACCAGCGCCAACAGCCGTGGGGA
AGCCAAGGAGACCCAAGGGGTCCAGGAGGTGGGCGCCCTCCATCCTTCGA
GAAGCTTCCCAGGCTCCTCTGCTTCTCTGTCTCATGCTCCCAGGCTGCAC
AGCAGGCAGGGAGGGAGGCAAGGCAGGGGAGTGGGGCCTGAGCTGAGCAC
TGCCCCCTCACCCCCCCACCACCCCTTCCCATTTCATCGGTGGGGACGTG
GAGAGGGTGGGGCGGGCTGGGGTTGGAGGGTCCCACCCACCACCCTGCTG
TGCTTGGGAACCCCCACTCCCCACTCCCCACATCCCAACATCCTGGTGTC
TGTCCCCAGTGGGGTTGGCGTGCATGTGTACATATGTATTTGTGACTTTT
CTTTGGATTTGTTTTGTGTTTTTGTTGACTAGTCCTGGAAATGTTTGAGG
CTAGACGGGGAGGGGCCAGGACCCACCCACTGCTCCTGGGGGATGAGGTC
CTGGTTTTAAAGCCCCGTCATTTCAAGCGGGTCGATCTTCCACATTCACT
GGAGAGACTCTCCCCACCTCTGTCTGGGTGGGGCGCGGACCCCTCACTGT
GCGCCTGTGCAGGGGGTGCTGGTGCACGTGGCAGTGTGGATTTCCAGTGG
TCACGGTCTTACTGTTTCAAGGTTTTTAAATAAGAAAACCAACCCTGCCT
TCGCCCATGCCCGCCCCTGCCCGCAGTTGCCAAAGAGCCGCCTTGTCGCT
GTGGGCGTCAGGGCTTGGCTGGCTCAGTGCACAACCCACAGTGGCCTTCA
GAGGCTCCTCCTGGGACTGGGAACCGCCGCAGGGCCAGGCGGACGGCGTG
AGGTTTGTGTTGGGGCTGGTTCTGCCCATGCTAGGGGGTGGGGGAGCTCC
CAGGACAGACCAGCCTTGTTTCTCATGTAATGCAGTGACGCTGTCATTAA
ACACGTGGATTCATGTGTGG

### SUB1

Suitably, the agent described herein reduces an expression and/or an activity of SUB1.

SUB1 (Activated RNA polymerase II transcriptional coactivator p15) is a general coactivator that functions cooperatively with TAFs and mediates functional interactions between upstream activators and the general transcriptional machinery. The nucleotide and protein sequences of SUB1 are publicly available (e.g., P53999, ENST00000265073.9 and NM_006713.3). An exemplary amino acid sequence is set forth in SEQ ID NO: 15. Thus, the SUB1 amino acid sequence may be a protein which is at least 70%, 75%, 80%, 85%, 90%, 95%, 96%, 97%, 98%, 99%, 99.5% or 100% identical to SEQ ID NO: 15 or a fragment or derivative thereof. An exemplary encoding nucleotide sequence (i.e., mRNA transcript) is set forth in SEQ ID NO: 16. Thus, the SUB1 nucleotide sequence may be a nucleic acid which is at least 70%, 75%, 80%, 85%, 90%, 95%, 96%, 97%, 98%, 99%, 99.5% or 100% identical to SEQ ID NO: 16 or a fragment or derivative thereof.

SUB1 protein
(SEQ ID NO: 15)
MPKSKELVSSSSSGSDSDSEVDKKLKRKKQVAPEKPVKKQKTGETSRALS
SSKQSSSSRDDNMFQIGKMRYVSVRDFKGKVLIDIREYWMDPEGEMKPGR
KGISLNPEQWSQLKEQISDIDDAVRKL
SUB1 nucleic acid
(SEQ ID NO: 16)
GCCCCATCACGTGACCGCAGCCCCAGCGCGGCGGGGCCGGCGTCTCCTGG
CTGCCGTCACTTCCGGTTCTCTGTCAGTCGCGAGCGAACGACCAAGAGGG
TGTTCGACTGCTAGAGCCGAGCGAAGCGATGCCTAAATCAAAGGAACTTG
TTTCTTCAAGCTCTTCTGGCAGTGATTCTGACAGTGAGGTTGACAAAAAG
TTAAAGAGGAAAAAGCAAGTTGCTCCAGAAAAACCTGTAAAGAAACAAAA
GACAGGTGAGACTTCGAGAGCCCTGTCATCTTCTAAACAGAGCAGCAGCA
GCAGAGATGATAACATGTTTCAGATTGGGAAAATGAGGTACGTTAGTGTT
CGCGATTTTAAAGGCAAAGTGCTAATTGATATTAGAGAATATTGGATGGA
TCCTGAAGGTGAAATGAAACCAGGAAGAAAAGGTATTTCTTTAAATCCAG
AACAATGGAGCCAGCTGAAGGAACAGATTTCTGACATTGATGATGCAGTA
AGAAAACTGTAAAATTCGAGCCATATAAATAAAACCTGTACTGTTCTAGT
TGTTTTAATCTGTCTTTTTACATTGGCTTTTGTTTTCTAAATGTTCTCCA
AGCTATTGTATGTTTGGATTGCAGAAGAATTTGTAAGATGAATACTTTTT
TTTAATGTGCATTATTAAAAATATTGAGTGAAGCTAATTGTCAACTTTAT
TAAGGATTACTTTGTCTGCCCACCACCTAGTGTAAAATAAAATCAAGTAA
TACAATCTTAACTGTTGTGGCCTTTTTTGATCATAAGAGTTGGTACTGTT
TAAGGCCAAAAGTAACAGTTTTTATAGATCTTTTAGTTTCAACTCAGCTT
TTACAATAAAAAGGATTTGTATTGCATTGAGTTTATAAACTTTTGGTTTG
TGAACTTCATATTTGATCTTTTCTCTTCCAATCAAATGTCTAGGCTTGTT
TGACTTCCACCCCCAATGGTTTTTCACTCTTTTTATTTACTTCATTTTCC
TTTAATAACTTAATCTCTTCATGTTCAGTTTTTACTTCACTCTTTATTCT
TTTCTTTGATTATGGTATGCTTATTTGGAAAGTCAGTGAAACTGTCAAAA
TGTTATCTCAATAAGATACTTATATGAGAACTACAATCACCGAATCTACT
GTATTCAATATTAGCAGATCTAATTTGATAAACAACATGGCTTGTGTGAA
AACTGAGCAGGTGTTTGTTTACCCATAGTGTTCTGTGTAGTTATTGCTTA
GTCTGCAGAAAATAATGACTTAGATGAGATGTCTGACTTGCTTTCACTTA
TTAAACATGTTCACCATGGGATGATGTCTGTAACATCAGATATTGTTCAA
CTAGACTAGGATTTAATAAAAATTGTGAAAGCTTACTGGCCTAACATTTT
ATTTTATAATATTGGGTATGAATTATATGTAGCCAGAGATGTCATTAAGC
TTTACTGTTATAGTAGGTAATATGGTTAGTTTGTAGGGAAAAGAGCATAT
GAGCACATGCTTGTGTATTTTGGCCTTTGCCCCAGTAGAACAGACCAATG
GCATTCTAGACTTGATGATACTAAGTTTTAGCAGACACTAGTAAGTGGTT
TGTATTTAACCATACTGATGAAGCAGACAGATTGAGGCACAGATTTTAGT
GGCTTTGTGGCAATAAATAGGGCATGGTGTGCCTTAGGAAAAGAATGTTT
ATAAAGGGAATTATAACTGAAATTAAAGGAGGCGGCAGTGAAGAGGAAAT
AATTCTCTTCTATCTAAATGATATACATATGATATTTTGAGATTTTTATA
ACAGCAGTGGAACACAATTCTAGGTAGAGTAGAAAAAGGAAAGTTTTAAA
GACATATAAAAGATTCTTGTTGACAAATTATTTTTGGTAGCAAATCTCAA
ATGGTTACCTGCTATTAAGGTCTGCCATATTAGAGTTTTGCACTATTTTG
CTACCAAGTTTGATTCATACATCTAAAACATTTTGTAGTTACTTGTCAAG
GACTTAATTTGAAAATCATTTGCCAGGCCACATAGTTATCAATTTTTTTT
TCTATCAGCTATTCTGTTGTATTTCTAAAACATTTTTTAGATGACTTTTT
AAAGTATATTTAGCAGTAACCTTATGAGGTTCAAATTGGTAAATCTCTTG
TAATTTAGCCTTCATCGAATAATAGGTACCAGTGTATTAAAAATGTGTAT
TTTTTGCAGCCCCTTGAACCAGAGTAGGTTCAGAGAAACTCCCAAAGTTT
GTACTTTAGACACATCATGCTTGATTGGTAACTTCCCTCCTTTTTTGGGG
AACATGTTTGTGTCCTATTAACTTAATTGGATAGATTTTTAAATATTTCT
TATTTTTGGCACACGGAAAGGGTAGTTCGAGTACAGAACTTTGATTTTTG
GTGTAGATGCAGAGGGAATGATGGGTAAATTTCCTAGGTTTATGTGAATT
TAGGGGGTGTATGCATTTTGAAACAATCTACTAACAGATGGTGCTGAAAT
CTATTACCTACATGTTTTCTAGTTGTTCAGCATTATGTTAATGAAGCCTC
CATATAAGGAGTGTTTCTCTGGCACAGTTGGTAAGTTGACTGCTAACTTC
ATTTAAATGTGTTACTGGATATGCAGTATACTGAAATTATTAATCAGTTT
GTGTATAGGAAAAGAGAACTGGGTTAAAAGCAAATTAACTTGTTCTGAAA
AGAAAGTATAGATTAATTTTGTTTTCTGTTTAAATTTTATCTCCTTGGTA
AAGATTTTTTTTTCCTGGGCAGAAAACTTGGCATTTTTAGGCGTAGATAC
CTTACCTTACAATGCCAAAATGAATTTAATTCCAGTACTCAGGTTTTTCC
CTTTAACAGACTCTATGTGTATCAGGGCTTTCTAATGGGTTTTTCCTCTT
CGTTTTTAAAATGTGAGTAGCATTTGACCAATTTCCAGTGCTCTTAGCAT
TTTACTTAAAGAACAACCACTACAAAAGAAAATCTTTGTAATTTGATTGT
CTTTTGCTTTGCTTCATTAATGCCTAAGAACTTAAGAATACTCCTACCTC
ATTAGCTACTCAAGATGCTGTGACGATCAAATCTATTCTACATAATGCGT
TTAGAAACAAAGACTTGGGTGAAAAATGAAATAAGTATATTCTGACTTGG
CTATTGAGGGGAAAATTCAGTATTAAGTGTTCCTCACAGGAGATATGTTA
GCAGAATACTATAAAAGTTTGAAATTTTTAAAAAGTAAAAGTACTTAAAT
TTAGGTATCTCTCCTGAAATTCTTTGCAGTTCATTTTTTATGGCAGTTAA
TCCAGTGAAACACTCAAAAGTTTTTTTTTTTTTAAAAGTGTTTTTCCAGA
TAAACTGTAGGGTGAACATTCACATAATCACAAATATGTAATTCTGTAAT
TGTGGAATGCTTGTATGCTTTGTTTTCGTACATCTTCCATGGAGATGTCT
GAATATAATACTCCATCTGTGAATATTTTAAATGTTGAAATAAAAGTAAG
AAATGTGAAAAAAAAAAAAAAA

### Further Anti-Cancer Treatments

Suitably, the methods described herein further include the step of administering a further therapeutic agent, such as a further anti-cancer agent, to the subject (i.e., in addition to the agent). As such, the agent described herein may be administered alone (i.e., monotherapy) or alternatively be administered in combination (e.g., concurrently, simultaneously, sequentially, successively or alternately) with the further therapeutic agent which aims to treat, ameliorate or prevent an ALT-dependent cancer or a disease, disorder or condition associated therewith.

In certain examples, the agent described herein may be co-administered with or formulated to be co-administered with (separately, simultaneously or sequentially) a further anti-cancer agent for the treatment of the ALT-dependent cancer. Additional cancer treatments (i.e., cancer treatments in addition to the agent that reduces an expression and/or an activity of one or more of TERF2IP, SAMHD1, CCDC93, CHTF18, ETAA1, ZMAT1, SIVA1, FZR1 and SUB1) for use in the methods described herein may include drug therapy, chemotherapy, antibody, nucleic acid and other biomolecular therapies, radiation therapy, surgery, nutritional therapy, relaxation or meditational therapy and other natural or holistic therapies, although without limitation thereto. Generally, drugs, biomolecules (e.g., antibodies, inhibitory nucleic acids such as siRNA) or chemotherapeutic agents are referred to herein as “anti-cancer therapeutic agents” or “anti-cancer agents”.

Suitably, the further anti-cancer agent is or comprises one or more of a chemotherapeutic agent, a radiation therapy, a molecularly targeted therapeutic agent and an immunotherapeutic agent.

As generally used herein, the term “chemotherapy” or “chemotherapeutic agent” broadly refers to a treatment or agent with a cytostatic or cytotoxic agent (i.e., a compound) to reduce or eliminate the growth or proliferation of undesirable cells, such as cancer cells. Accordingly, the terms can refer to a cytotoxic or cytostatic agent used to treat a proliferative disorder, for example cancer. The cytotoxic effect of the agent can be, but is not required to be, the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect.

Exemplary chemotherapeutic agents include, but are not limited to, alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin, oxaliplatin, nedaplatin, triplatin tetranitrate, phenanthriplatin, picoplatin, satraplatin and lipoplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

The term “radiation therapy” or “radiotherapy” used herein refers to the medical use of ionizing radiation, generally as part of cancer treatment, to control or destroy malignant cells. It can also be used as part of adjuvant therapy to prevent tumour recurrence after surgery to remove a primary malignant tumour.

In particular examples, the further anti-cancer agent is or comprises chemoradiotherapy (CRT), which refers to the combined administration of both chemotherapy and radiotherapy as an anticancer treatment to a subject. The chemotherapy and radiotherapy can be concurrent or sequential. The term “radiochemotherapy” may be used interchangeably herein with the term “chemoradiotherapy”. CRT may include any combination of type, form or modality of chemotherapy and radiotherapy as are known in the art, such as those described herein.

As used herein, “molecularly targeted therapy” or “molecularly targeted therapeutic agent” refers to a therapy that targets a particular class of proteins involved in cancer growth or signalling. It is envisaged that such agents may include antibodies or fragments thereof and small molecules.

In some examples, the further anti-cancer agent described herein is or comprises an inhibitor of a kinase, such as a tyrosine kinase or a serine-threonine kinase. The term “tyrosine kinase” refers to enzymes which are capable of transferring a phosphate group from ATP to a tyrosine residue in a protein. Phosphorylation of proteins by tyrosine kinases is an important mechanism in signal transduction for regulation of enzyme activity and cellular events such as cell survival or proliferation. Tyrosine kinases include receptor tyrosine kinases and non-receptor tyrosine kinases. AATK, ABL, ABL2, ALK, AXL, BLK, BMX, BTK, CSF1 R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYR03, YES1, and ZAP70.

Insofar as they relate to cancer, immunotherapy or immunotherapeutic agents use or modify the immune mechanisms of a subject so as to promote or facilitate treatment of a cancer. In this regard, immunotherapy or immunotherapeutic agents used to treat cancer include cell-based therapies, antibody therapies (e.g., anti-PD1 or anti-PDL1 antibodies) and cytokine therapies. These therapies all exploit the phenomenon that cancer cells often have subtly different molecules termed cancer antigens on their surface that can be detected by the immune system of the cancer subject. Accordingly, immunotherapy is used to provoke the immune system of a cancer patient into attacking the cancer's cells by using these cancer antigens as targets.

Non-limiting examples of immunotherapy or immunotherapeutic agents include adalimumab, alemtuzumab, basiliximab, belimumab, bevacizumab, BMS-936559, brentuximab, certolizumab, cituximab, daclizumab, eculizumab, ibritumomab, infliximab, ipilimumab, lambrolkizumab, mepolizumab, MPDL3280A muromonab, natalizumab, nivolumab, ofatumumab, omalizumab, pembrolizumab, pexelizumab, pidilizumab, rituximab, tocilizumab, tositumomab, trastuzumab, ustekinumab, abatacept, alefacept and denileukin diftitox. In particular preferred embodiments, the immunotherapeutic agent is an immune checkpoint inhibitor, such as an anti-PD1 antibody (e.g., pidilizumab, nivolumab, lambrolkizumab, pembrolizumab), an anti-PDL1 antibody (e.g., BMS-936559, MPDL3280A) and/or an anti-CTLA4 antibody (e.g., ipilimumab).

In some examples, the further anti-cancer agent is or comprises an inhibitor or antagonist of an ALT mechanism. Such inhibitors or antagonists may target (e.g., modulate or inhibit the expression and/or activity of) known therapeutic targets of ALT, such as SP100, TRF1 (e.g., T271 and/or T371 phosphorylation thereof), MMS21 SUMO ligase, TSPYL5, FANCM/FAAP24, FANCM/BTR (e.g., PIP-199), ATRX/DAXX, NRSC/F, SMARCAL1, FA core complex, FANCM-interacting proteins, TRF2, PARP, HOP2-MND1 heterodimer, DMC1, RAD51AP1, NBS1, MRN (e.g., Mirin), POLD3/4, XPF, WEE1 (e.g., MK-1775), PKMYT1 (e.g., RP-6306) and ATM (e.g., AZD0156).

### Administration

Suitably, the various agents, anti-cancer agents or cancer treatments described herein are administered to a subject as a pharmaceutical composition comprising a pharmaceutically-acceptable carrier, diluent or excipient. In this regard, any dosage form and route of administration, such as those provided therein, may be employed for providing a subject with the composition of the present disclosure.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, liposomes and other lipid-based carriers, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991), which is incorporated herein by reference.

Any safe route of administration may be employed for providing a patient with the composition of the present disclosure. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present disclosure suitable for oral or parenteral administration may be presented as discrete units such as capsules, sachets or tablets each containing a pre-determined amount of one or more therapeutic agents of the present disclosure, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the present disclosure with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present disclosure, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

It is envisaged that the various agents, anti-cancer agents or cancer treatments described herein can be formulated as discrete doses, such as in the form of a kit. Such a kit may further comprise a package insert comprising printed instructions for simultaneous, concurrent, sequential, successive, alternate or separate use of the agents in the treatment, amelioration and/or prevention of cancer, as described herein, in a patient in need thereof. Accordingly, the aforementioned kits are suitably for use in a method of treating, ameliorating and/or preventing cancer, inclusive of one or more symptoms, consequences, sequelae or complications thereof, as described herein.

Alternatively, the various therapeutic agents described herein can be formulated together in a composition that optionally includes a pharmaceutically acceptable carrier, excipient or diluent.

### Screening Methods

Also described herein are methods of identifying or producing an agent for use in the treatment of an ALT-dependent cancer in a subject. Suitably, such methods include contacting a cell, more particularly an ALT-dependent cell or even more particularly an ALT-dependent cancer cell, that expresses one or more of TERF2IP, SAMHD1, CCDC93, CHTF18, ETAA1, ZMAT1, SIVA1, FZR1 and SUB1 with a candidate agent and determining whether the candidate agent either modulates the expression and/or an activity of one or more of TERF2IP, SAMHD1, CCDC93, CHTF18, ETAA1, ZMAT1, SIVA1, FZR1 and SUB1. Further provided herein are agents produced according to such screening methods.

Suitably, the candidate agent, at least partly, reduces, eliminates, suppresses or inhibits the expression and/or the activity of one or more of TERF2IP, SAMHD1, CCDC93, CHTF18, ETAA1, ZMAT1, SIVA1, FZR1 and SUB1. The expression (inclusive of nucleic acid and/or protein expression) and/or activity of TERF2IP, SAMHD1, CCDC93, CHTF18, ETAA1, ZMAT1, SIVA1, FZR1 and SUB1 can be assessed in the cells treated with the candidate agent using methods known in the art, such as those described herein.

The terms, “lower”, “reduced” and “decreased”, as used herein refer to a lower amount or level of TERF2IP, SAMHD1, CCDC93, CHTF18, ETAA1, ZMAT1, SIVA1, FZR1 or SUB1 nucleic acid or protein expression and/or activity, such as in the cell, when compared to a control or reference level or amount (e.g., a cell not contacted with the candidate agent). The activity level and/or the expression level of the nucleic acid or protein of these mediators of an ALT mechanism may be relative or absolute (i.e., relatively or absolutely lower, reduced or decreased). In some examples, the activity level and/or the expression level of TERF2IP, SAMHD1, CCDC93, CHTF18, ETAA1, ZMAT1, SIVA1, FZR1 or SUB1 nucleic acid or protein is lower, reduced or decreased if its level is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level of expression or activity of TERF2IP, SAMHD1, CCDC93, CHTF18, ETAA1, ZMAT1, SIVA1, FZR1 or SUB1 in a control or reference cell, such as in the absence of the candidate agent, or a reference or threshold level.

The terms, “higher”, “elevated” and “increased”, as used herein refer to a greater amount or level of TERF2IP, SAMHD1, CCDC93, CHTF18, ETAA1, ZMAT1, SIVA1, FZR1 or SUB1 nucleic acid or protein expression and/or activity, such as in the cell, when compared to a control or reference level or amount (e.g., a non-ALT cell). The activity level and/or the expression level of the nucleic acid or protein of these mediators of an ALT mechanism may be relative or absolute (i.e., relatively or absolutely higher, elevated or increased). In some examples, the activity level and/or the expression level of TERF2IP, SAMHD1, CCDC93, CHTF18, ETAA1, ZMAT1, SIVA1, FZR1 or SUB1 nucleic acid or protein is higher, elevated or increased if its level is more than about 1%, 5%, 10%, 15%, 20%, 30%, 40%, 50%, 60% or 70%, or even more than about 80%, 90%, 100%, 125%, 150%, 175%, 200%, 300%, 400% or 500% greater than the level of expression or activity of TERF2IP, SAMHD1, CCDC93, CHTF18, ETAA1, ZMAT1, SIVA1, FZR1 or SUB1 in a control or reference cell (e.g., a non-ALT cell) or when compared to reference or threshold level.

The method may include a further step that measures or detects a change in one or more biological activities of TERF2IP, SAMHD1, CCDC93, CHTF18, ETAA1, ZMAT1, SIVA1, FZR1 and/or SUB1 in response to the candidate agent(s). In particular examples, such screening methods further include the step of determining whether the candidate agent positively modulates a characteristic and/or an activity of the cell.

According to particular examples, the present method further includes the step of determining whether the candidate agent modulates the viability and/or growth of the cell, such as by those methods hereinbefore described. Suitably, the candidate agent, at least partly, reduces the viability and/or growth of the cell.

For some examples, the candidate agent decreases the viability and/or growth of the cell to less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level of viability and/or growth in a control or reference cell in the absence of the candidate agent.

For some examples, the present method further includes the step of determining whether the candidate agent modulates a level of genomic stability at one or more telomeres in the cell and/or a level of ALT activity in the cell. Suitably, the candidate agent, at least partly, increases the level of genomic stability at one or more telomeres in the cell and/or at least partly, reduces the level of ALT activity in the cell.

In some examples, the candidate agent increases the level of genomic stability at one or more telomeres in the cell by more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% above the level of genomic stability in a control or reference cell in the absence of the candidate agent.

In other examples, the candidate agent decreases the level of ALT activity in the cell to less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level of ALT activity in a control or reference cell in the absence of the candidate agent.

The effect of a candidate agent may be upon TERF2IP, SAMHD1, CCDC93, CHTF18, ETAA1, ZMAT1, SIVA1, FZR1 and/or SUB1 expression, inclusive of intracellular expression, secretion, protein expression and gene expression thereof. It will be appreciated that in certain cells, such as ALT-dependent cancer cells although without limitation thereto, candidate agents may cause or facilitate a removal, loss and/or down-regulation of TERF2IP, SAMHD1, CCDC93, CHTF18, ETAA1, ZMAT1, SIVA1, FZR1 and/or SUB1 expression and secretion. Thus, some examples may include removal, loss and/or down-regulation of TERF2IP, SAMHD1, CCDC93, CHTF18, ETAA1, ZMAT1, SIVA1, FZR1 and/or SUB1 expression at or near the cell-surface, intracellularly and extracellularly. In particular examples, protein expression of TERF2IP, SAMHD1, CCDC93, CHTF18, ETAA1, ZMAT1, SIVA1, FZR1 and/or SUB1 may be detected or measured by western blotting, flow cytometry, immunoprecipitation, immunocytochemistry or immunohistochemistry, typically by way of an antibody or antibody fragment which binds TERF2IP, SAMHD1, CCDC93, CHTF18, ETAA1, ZMAT1, SIVA1, FZR1 and/or SUB1, as hereinbefore described. In particular examples, TERF2IP, SAMHD1, CCDC93, CHTF18, ETAA1, ZMAT1, SIVA1, FZR1 and/or SUB1 gene expression may be measured by nucleic acid sequence amplification (e.g., PCR inclusive of quantitative and semi-quantitative PCR) or nucleic acid hybridization techniques, such as Northern blotting.

Suitably, the cell is or comprises an ALT-dependent cell. In various examples, the cell is or comprises an ALT-dependent cancer cell, such as that described herein. In particular examples, the ALT-dependent cancer cell is grown in a cell culture, a 3D culture or a xenograft model, such as a patient-derived xenograft model.

The candidate molecule may be a protein (inclusive of peptides, aptamers, antibodies and antibody fragments), a nucleic acid (inclusive of gene therapy and inhibitory nucleic acid molecules, such as ribozymes, RNAi, miRNA, siRNA and antisense oligonucleotides, although without limitation thereto), a lipid, a carbohydrate, a small organic molecule or any combination of these (e.g., a glycoprotein, a lipoprotein, a peptide-nucleic acid etc). In some examples, the agent is or comprises an immunoglobulin, such as an antibody or fragment thereof, or a small molecule. In examples relating to antibody inhibitors, the antibody may be polyclonal or monoclonal, native or recombinant. In various examples, the candidate molecule is an inhibitory nucleic acid molecule.

Typically, the inhibitory activity of candidate agents may be assessed by in vitro and/or in vivo assays that detect or measure the expression levels and/or activity of TERF2IP, SAMHD1, CCDC93, CHTF18, ETAA1, ZMAT1, SIVA1, FZR1 and/or SUB1 in the presence of the candidate agent.

The modulators can be small organic molecule inhibitors. This may involve screening of large compound libraries, numbering hundreds of thousands to millions of candidate inhibitors (chemical compounds including synthetic, small organic molecules or natural products, such as inhibitory peptides or proteins) which may be screened or tested for biological activity at any one of hundreds of molecular targets in order to find potential new drugs, or lead compounds. Screening methods may include, but are not limited to, computer-based (“in silico”) screening and high throughput screening based on in vitro assays.

Typically, the active compounds, or “hits”, from this initial screening process are then tested sequentially through a series of other in vitro and/or in vivo tests to further characterize the active compounds. A progressively smaller number of the “successful” compounds at each stage are selected for subsequent testing, eventually leading to one or more drug candidates being selected to proceed to being tested in human clinical trials.

At the clinical level, screening a candidate agent may include obtaining samples from test subjects before and after the subjects have been exposed to a test compound. The levels in the samples of TERF2IP, SAMHD1, CCDC93, CHTF18, ETAA1, ZMAT1, SIVA1, FZR1 and/or SUB1 or one or more markers of TERF2IP, SAMHD1, CCDC93, CHTF18, ETAA1, ZMAT1, SIVA1, FZR1 and/or SUB1 activity or signalling may then be measured and analysed to determine whether the levels and/or activity thereof changes after exposure to a candidate agent. By way of example, protein product levels in the samples may be determined by mass spectrometry, western blot, ELISA, electrochemistry and/or by any other appropriate means known to one of skill in the art.

In this regard, candidate agents that are identified of being capable of reducing, eliminating, suppressing or inhibiting the expression level and/or activity of TERF2IP, SAMHD1, CCDC93, CHTF18, ETAA1, ZMAT1, SIVA1, FZR1 and/or SUB1 may then be administered to patients who are suffering from an ALT-dependent cancer. For example, the administration of a candidate agent which inhibits or decreases the activity and/or expression of TERF2IP, SAMHD1, CCDC93, CHTF18, ETAA1, ZMAT1, SIVA1, FZR1 and/or SUB1 may treat the ALT-dependent cancer and/or decrease the risk or progression of the ALT-dependent cancer, if the increased activity and/or expression of TERF2IP, SAMHD1, CCDC93, CHTF18, ETAA1, ZMAT1, SIVA1, FZR1 and/or SUB1 is responsible, at least in part, for the progression and/or onset of said ALT-dependent cancer.

It is also contemplated that the candidate agent may be rationally designed or engineered de novo based on desired or predicted structural characteristics or features that indicate the candidate modulator could block or inhibit one or more biological activities of TERF2IP, SAMHD1, CCDC93, CHTF18, ETAA1, ZMAT1, SIVA1, FZR1 and/or SUB1. In other examples, the candidate agent may be identified by screening a library of molecules without initial selection based on desired or predicted structural characteristics or features that indicate the candidate modulator could block or inhibit one or more biological activities of TERF2IP, SAMHD1, CCDC93, CHTF18, ETAA1, ZMAT1, SIVA1, FZR1 and/or SUB1. Such libraries may comprise randomly generated or directed libraries of proteins, peptides, nucleic acids, recombinant antibodies or antibody fragments (e.g., phage display libraries), carbohydrates and/or lipids, libraries of naturally-occurring molecules and/or combinatorial libraries of synthetic organic molecules.

Non-limiting examples of techniques applicable to the design and/or screening of candidate agents may employ X-ray crystallography, NMR spectroscopy, computer assisted screening of structural databases, computer-assisted modelling or biochemical or biophysical techniques which detect molecular binding interactions, as are well known in the art.

Biophysical and biochemical techniques which identify molecular interactions include competitive radioligand binding assays, co-immunoprecipitation, fluorescence-based assays including fluorescence resonance energy transfer (FRET) binding assays, electrophysiology, analytical ultracentrifugation, label transfer, chemical cross-linking, mass spectroscopy, microcalorimetry, surface plasmon resonance and optical biosensor-based methods, such as provided in Chapter 20 of CURRENT PROTOCOLS IN PROTEIN SCIENCE Eds. Coligan ei 1, (John Wiley & Sons, 1997) Biochemical techniques such as two-hybrid and phage display screening methods are provided in Chapter 1 of CURRENT PROTOCOLS IN PROTEIN SCIENCE Eds. Coligan et al, (John Wiley & Sons, 1997).

Accordingly, an initial step of the method may include identifying a plurality of candidate molecules that are selected according to broad structural and/or functional attributes, such as an ability to bind one or more of TERF2IP, SAMHD1, CCDC93, CHTF18, ETAA1, ZMAT1, SIVA1, FZR1 and SUB1 or an encoding nucleic acid thereof.

According to various examples, the present method further includes one or more of the steps of:
selecting the candidate agent that modulates, and more particularly decreases, the expression and/or the activity of one or more of TERF2IP, SAMHD1, CCDC93, CHTF18, ETAA1, ZMAT1, SIVA1, FZR1 and SUB1;
selecting the candidate agent that, at least partly, increases the level of genomic stability at one or more telomeres in the cell and/or at least partly, reduces the level of ALT activity in the cell;
selecting the candidate agent that, at least partly, reduces the viability and/or growth of the cell;
isolating or purifying the candidate agent;
formulating the candidate agent into a pharmaceutical formulation; and
adding the candidate agent or the pharmaceutical formulation to packaging and/or a container.

So that preferred embodiments of the present disclosure may be fully understood and put into practical effect, reference is made to the following non-limiting examples.

### EXAMPLES

### Example 1

The mechanisms that underpin cancer cell immortality (telomere maintenance mechanisms, TMMs) provide an opportunity in cancer drug development. Most cancers utilise one or the other of two known telomere maintenance mechanisms (TMMs); either telomerase or an Alternative Lengthening of Telomeres (ALT) mechanism. The objective of this Example is to identify new molecular targets that may be exploited in the development of new treatments for ALT cancers.

The mechanisms that underpin cancer cell immortality function to maintain the length of DNA-protein structures at the ends of chromosomes called telomeres. Most commonly, telomere maintenance in cancer cells is mediated by an enzyme called telomerase that synthesises telomeric DNA. The other TMMs that operate in cancer cells are collectively referred to Alternative Lengthening of Telomeres (ALT). One way ALT mediates telomere maintenance is through DNA recombination events that occur as chromosomes replicate just before cell division. Although ALT is less commonly activated in human cells than telomerase, ALT tends to be more prevalent in aggressive and difficult to treat cancers, such as bone and soft-tissue sarcomas, certain types of paediatric brain tumours and pancreatic neuroendocrine cancers. There are currently no cancer treatments available that target or inactivate ALT.

### Materials & Methods

### Cell Line Panel

Telomere maintenance mechanisms were determined for a panel of 976-cancer derived cell lines using a combination of semi-quantitative assays for detection of telomerase, phenotypic markers of ALT, telomere content and telomere length maintenance. All cell lines were subject to the qTRAP assay, C-circle assay and telomere content analysis by qPCR. Cell lines that gave positive results in the C-circle assay (greater than or equal to 2% of control cell line DOS16) were further investigated in APB, ALT-FISH and TRF assays. Cell lines that were negative or had low levels of telomerase (including those that were C-circle positive) were also subject to long-term culture for longitudinal analysis of TMM. ALT cell lines were defined as those that were had no telomerase at any time point, long telomeres and C-circles greater than 2% DOS16 when calculated as an average across all time points assayed. Most ALT cell lines were also positive in the ALT-FISH assays, while a lower proportion were also positive in the APB assay.

### Cell Culture and Sample Preparation

TMM assays for telomerase activity, telomere length and C-circles used snap frozen cell pellets prepared from cultured cells. The cells were cultured using standard procedures and reagents appropriate for each cell line. Cells harvested from culture were centrifuged, washed in ice-cold PBS twice then, after a final centrifugation step, the supernatant was removed and the cell pellet stored at −80° C. until it was used in TMM assays.

Quantitative Telomeric Repeat Amplification Protocol (qTRAP)

Telomerase activity was measured using the PCR-based Quantitative Telomeric Repeat Amplification Protocol (qTRAP) assay. Cell pellets were lysed in CHAPS buffer (10 mM Tris-Cl pH 8.0, 1 mM MgCl2, 1 mM EGTA, pH 8.0, 10% v/v Glycerol, 0.5% w/v CHAPS detergent, 1 mM phenylmethylsulfonyl fluoride (PMSF), 15 mM β-mercaptoethanol), and protein concentration in the lysate measured using the Pierce BCA Tube Protein Assay Kit (Reducing Agent Compatible) (Thermo Fisher Scientific). Cell lysates containing 5 μg total protein were added into the telomere extension mix (1 mM dNTP mix (Thermo Fisher Scientific), 150 ng M2 primer, 40 units RNaseOUT™ Recombinant Ribonuclease Inhibitor (10777019, Thermo Fisher Scientific), 20 mM 10 mM Tris-Cl pH 8.0, 1.5 mM MgCl2, 63 mM KCl, 0.05% v/v Tween-20, 1 mM EGTA pH=8.0), incubated at 30° C. for 35 minutes, then heated at 95° C. for 20 minutes. Telomeric sequence was amplified in a qPCR assay using SensiFAST™ SYBR® No-ROX Kit (Bioline) using 1 μL of telomere extension product, 50 ng M2 primer and 25 ng ACX primer for a 10 μL reaction system amplified on the LightCycler® 96 (Roche) machine with PCR settings of 95° C. for 5 minutes, 45 cycles of 95° C. for 15 seconds, 60° C. for 60 seconds and 72° C. for 30 seconds, finishing with a melting curve cycle. A standard curve was generated by assaying serial dilutions of lysate from the MCF7 cell line. Each reaction (including controls) was run as technical triplicates in the qPCR step. Results were analysed using the ΔΔCt method using the LightCycler® 96 system software, and normalised to undiluted MCF7.

### C-Circle Assay

The C-circle assay was performed as previously described (1). Briefly, cell pellets were resuspended in an appropriate volume of QCP buffer and incubated with shaking (200 g rpm) at 56° C. for 1 hour, then at an increased temperature of 70° C. for 20 minutes. C-circles were quantified from an aliquot of QCP lysate containing the equivalent of 20 ng DNA. Following amplification of C-Circles with Φ29 DNA polymerase (New England Biolabs) at 30° C. for 8 hours, the reaction product was applied to a Biodyne B 0.45 μm nylon membrane (Pall) through vacuum absorption and air drying. The DNA was cross-linked onto the membrane then hybridised to a telomere probe labelled with ATP [γ32P] (Perkin Elmer, #NEG502A250UC). C-circle signals were analysed using ImageQuant TL software (GE Healthcare Life Sciences) and quantified by subtraction of a Φ29-negative control for each cell line and normalising to DOS16 cell line.

### Detection of ALT-Associated PML Bodies (APBs)

The APB assay was performed on cells grown in 4-well LabTek II chamber slides (Thermo Scientific) essentially as described (2), incubating in primary anti-PML goat antibody (Santa Cruz) (1:200 diluted in ABDIL buffer) at 4° C. overnight, then washing three times in PBST buffer (PBS and 0.1% v/v Tween-20) and incubating with 300 μL secondary antibody donkey anti-goat (Invitrogen) (1:500 diluted in ABDIL buffer) at room temperature for 1 hour. After further wash steps, the cells were fixed again with 500 μL 4% v/v formaldehyde then dehydrated by a graded ice-cold ethanol series and air-dried. The cells spots were overlaid with 200 μL working stock of peptide nucleic acid (PNA)-labelled probe (0.3 μg/ml Alexa488-OO-(CCCTAA)3), heated at 80° C. for 3 minutes and incubated at room temperature for overnight hybridisation. The next day, the slides were washed, incubated with 200 μL 100 ng/mL DAPI for 10 minutes at room temperature, washed again, air dried and mounted on slides with Prolong Gold Antifade (Invitrogen) overnight at room temperature for microscopy. The cells were imaged with an Axio-Imager M1 (Carl Zeiss) microscope at the CMRI Advanced Imaging Facility. Co-localisation of PML and telomere signals were analysed using ZEN2 software.

qPCR Assay for Measurement of Relative Telomere Content (TC)

Relative telomere content was measured using a modified version of the method described by Dahlgren et al., 2018, using a multicopy region of GAPDH gene as an internal control (3, 4). QCP lysate containing 5 ng DNA was used in each reaction. qPCR reactions were run in triplicate in strip tubes on a Rotor Gene Q (Qiagen). Telomere content values for each cell line were quantified relative to the control cell line HT1080-hTR, which was assigned the value of 8.0 to reflect mid-range telomere length.

### Terminal Restriction Fragment (TRF) Assay

The TRF assay was performed using genomic DNA extracted using the QIAmp DNA Blood Mini Kit. Twenty μg DNA was digested with restriction enzymes HinF1 and Rsa1, then precipitated and resuspended in 10 mM Tris (pH 7.6). Digested DNA (1 μg) was fractionated by pulse-field gel electrophoresis at 6V/sec with initial switching time of 1 and secondary time of 6 at 140 C for 13-16 hr. The gel was hybridised to a (CCCTAA) 4 telomeric probe labelled with Y32P-ATP using T4 kinase. After washing, the gel was exposed to a phosphoscreen then imaged on a Typhoon Imager. Mean telomere length was measured as described (5).

### ALT-FISH Assay

The ALT-FISH assay was performed on cell lines cytocentrifuged onto microscope slides. Cells on the slides were hybridised to a fluorescent labelled G-rich telomere probe and DAPI stain as described (6). The cells were imaged with an Axio-Imager M1 (Carl Zeiss) microscope at the CMRI Advanced Imaging Facility and foci quantified using Cell Profiler software.

### Gene Dependency Analysis

Gene dependencies specific for ALT cancer cells were identified through bioinformatic analysis of CRISPR/Cas9 whole genome knock-out data generated by the WSI and BI. CERES scores, indicating the strength of gene dependencies, were available for 518 out of the 976 cell lines that were characterised for TMM. Differential comparison of CERES dependency scores of ALT vs other cancer cell lines was performed using data from 17,846 genes using the limma package in R Studio with tissue type set as a covariate (7). The p value generated via t-test was corrected for multiple comparisons by the Benjamini-Hochberg (BH) method to acquire an adjusted p value (FDR)(8). ALT gene dependencies were defined by negative CERES scores, with adjusted p value<0.01.

Gene Dependency Validation by siRNA-Mediated Gene Suppression

Novel gene dependencies identified in differential analysis of CERES scores were verified by siRNA-mediated gene suppression of all target genes in a set of 8 cell lines. The cell line panel comprised of 4 ALT cell lines (Hs746T, SKNFI, CAL72, SAOS2) and 4 telomerase-positive cell lines (HGC27, SKNSH, 143B, SJSA1) that were matched for tissue type to enable direct comparison of the effects of gene suppression in ALT cell lines and telomerase cell lines. Target genes SAMHD1 and TERF2IP were further tested by siRNA-mediate gene suppression in two additional ALT cell lines (Hu09 and MUG_CHO_1) and two additional TEL cell lines (Ht1080 and RD). The siRNA molecules tested and their respective efficacies are provided in Table 1 below.

The cells were grown to semi-confluence in tissue culture flasks, then split into 6 well and 96 well plates and transfected with 25 nM siRNA (Thermo Fisher Scientific) in Opti-MEM® media with 0.25% (v/v) RNAiMAX (Thermo Fisher Scientific) for 48 hrs. Transfected cells in the 6 well plate were harvested for qRT-PCR analysis of gene expression, while cells in the 96 well plate were monitored for proliferation over 5-10 days using live cell imaging in an Incucyte instrument to measure percentage cell confluency. ALT gene dependencies were verified by reduced growth of ALT cells transfected with siRNA targeting candidate genes relative to control siRNA. The specificity of growth inhibition in ALT cells was evaluated relative to the effects of siRNA-mediated gene suppression in telomerase-positive cells.

TABLE 1
siRNA for suppression of ALT target genes

| | | | | Sense sequence | | |
|---|---|---|---|---|---|---|
| | | Manu- | | (5′->3′) | SEQ | Gene |
| | | fac- | | Anti-sense | ID | Suppression |
| Gene | Species | turer | siRNA Lab ID | sequence (5′->3′) | NO: | efficiency |
| CCDC93 | Human | Thermo | siCCDC93_1/a | GUGCCGAGCUAAUACAGUAtt | 20 | 20%-76% |
| | | | | UACUGUAUUAGCUCGGCACgg | 21 | |
| CCDC93 | Human | Thermo | siCCDC93_2/b | CAAUGACUCAUGACGAAGAtt | 22 | 28%-88% |
| | | | | UCUUCGUCAUGAGUCAUUGca | 23 | |
| CCDC93 | Human | Thermo | siCCDC93_3/c | GAUGAUGACUUCAUAAAGAtt | 24 | 20%-81% |
| | | | | UCUUUAUGAAGUCAUCAUCct | 25 | |
| FZR1 | Human | Thermo | siFZR1_1/a/(I) | GGAUUAACGAGAAUGAGAAtt | 26 | 60%-99% |
| | | | | UUCUCAUUCUCGUUAAUCCtg | 27 | |
| FZR1 | Human | Thermo | siFZR1_ | GCAAAACCCGUUCGACAAAtt | 28 | 18%-88% |
| | | | 2/b/(II) | UUUGUCGAACGGGUUUUGCta | 29 | |
| SAMHD1 | Human | Thermo | siSAMHD1_1/a | GUAUCGCAUUUCUACAGCAtt | 30 | 82%-100% |
| | | | | UGCUGUAGAAAUGCGAUACtt | 31 | |
| SAMHD1 | Human | Thermo | siSAMHD1_ | CGCAACUCUUUACACCGUAtt | 32 | 75%-99% |
| | | | 2/b/(I) | UACGGUGUAAAGAGUUGCGag | 33 | |
| SAMHD1 | Human | Thermo | siSAMHD1_ | GUGCUAAACCCAAAGUAUUtt | 34 | 74%-99% |
| | | | 4/d/(II) | AAUACUUUGGGUUUAGCACtg | 35 | |
| SIVA1 | Human | Thermo | siSIVA1_1/a | GCAGUGACAUGUACGAGAAtt | 36 | 73%-99% |
| | | | | UUCUCGUACAUGUCACUGCag | 37 | |
| SIVA1 | Human | Thermo | siSIVA1_2/b | CCUAAUGACAGAAUGAAUAtt | 38 | 70%-100% |
| | | | | UAUUCAUUCUGUCAUUAGGat | 39 | |
| TERF2IP | Human | Thermo | siTERF2IP_1/a | GGCAGUUAAUGGAGAAGUUtt | 40 | 63%-96% |
| | | | | AACUUCUCCAUUAACUGCCga | 41 | |
| TERF2IP | Human | Thermo | siTERF2IP_ | GAGCCCUCCUGAUUUUGAAtt | 42 | 50%-95% |
| | | | 3/c/(I) | UUCAAAAUCAGGAGGGCUCtc | 43 | |
| TERF2IP | Human | Thermo | siTERF2IP_ | AGAAAUCCAGGAGAAUGAAtt | 44 | 76%-93% |
| | | | 4/d(II) | UUCAUUCUCCUGGAUUUCUtc | 45 | |

| | | |
|---|---|---|
| Silencer™ | Thermo | siNC |
| Select Negative | | |
| Control No. 2 siRNA | | |

### Demonstration of Gene Suppression by RT-PCR

Effective gene suppression induced by siRNA transfection was confirmed by qRT-PCR analysis. RNA was extracted from cells harvested from 6 well plates using RNeasy® Mini Kit (Qiagen) following the kit manual. cDNA was generated using SuperScript™ III Reverse Transcriptase kit (Invitrogen) and amplified in a qPCR reaction using SensiFAST™ SYBR® No-ROX Kit (Bioline) based on the manufacturer's manual with 1 μL of cDNA product from (ii) as template, 1 μL forward/reverse primer (10 μM) in a 10 μL reaction system. The reactions were performed on a LightCycler® 96 (Roche) machine with PCR settings of 95° C. for 5 minutes, 40 cycles of 95° C. for 25 seconds, 60° C. for 30 seconds and 72° C. for 45 seconds, finishing with a melting curve cycle. Each reaction was run as technical triplicates. Results were analysed using the ΔΔCt method using the LightCycler® 96 system software.

### Results

To identify molecular pathways that may be exploited for therapeutic targeting in cancers with ALT, the present inventors characterised TMM in 976 cancer-derived cell models (cell lines), then utilised bioinformatic methods to identify genes essential for survival of ALT cancer cells. The bioinformatic analysis utilised data from a TMM screen of 976 cell lines, together with gene dependency data on an overlapping set of cell lines which has been made publicly available by the WSI and BI.

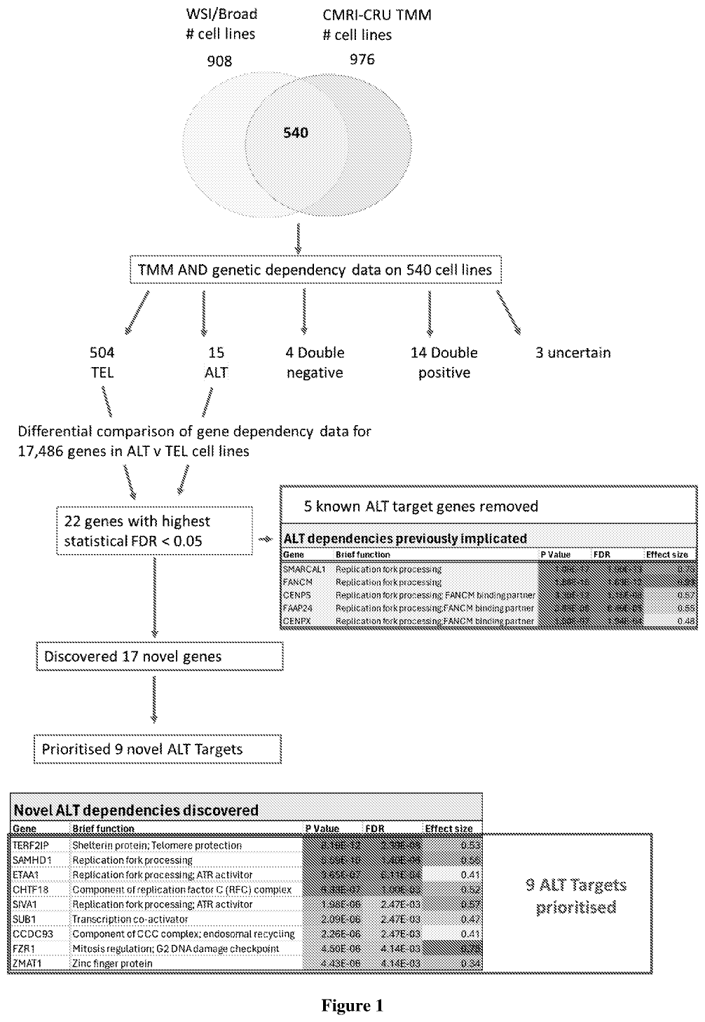

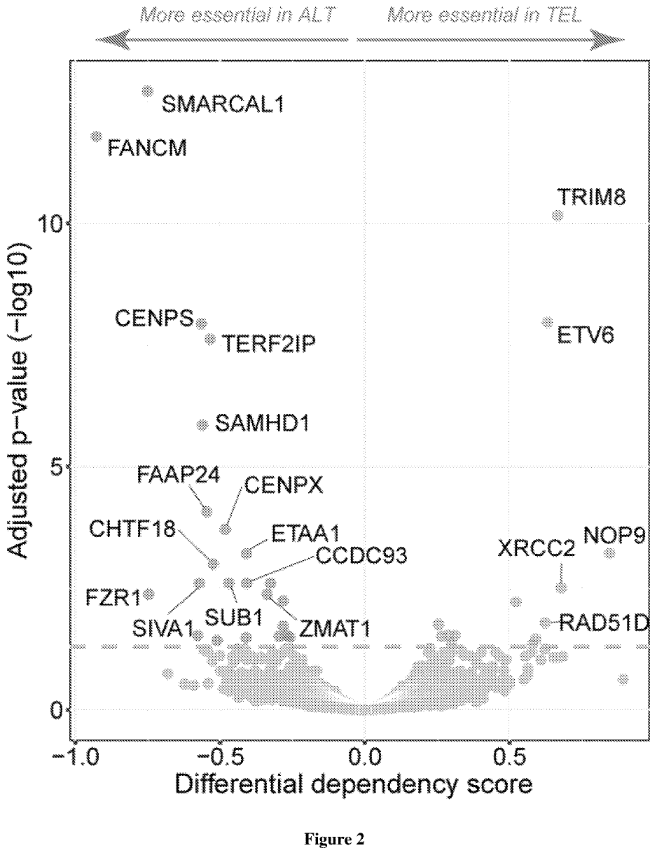

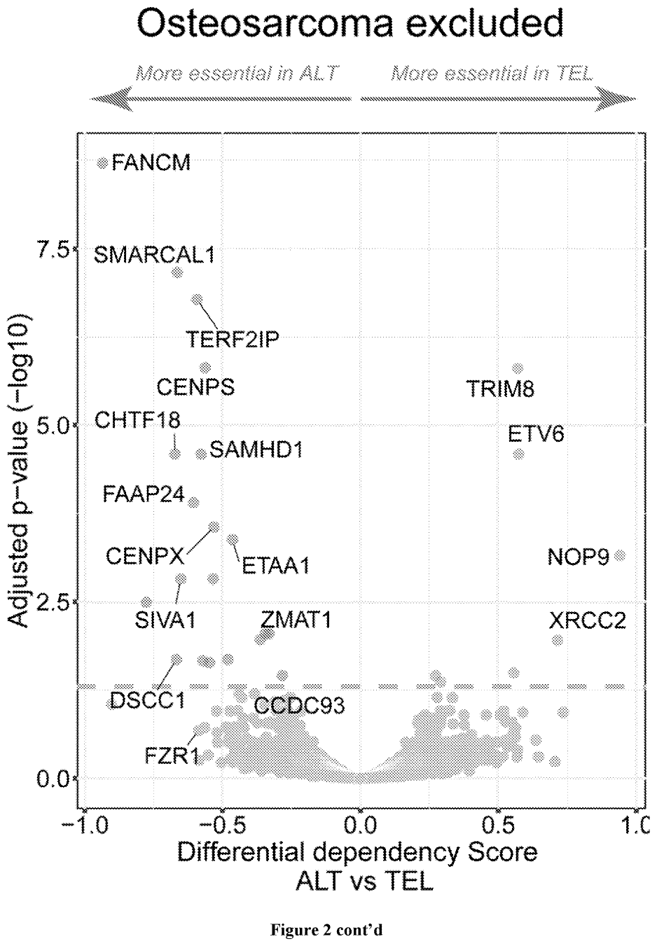

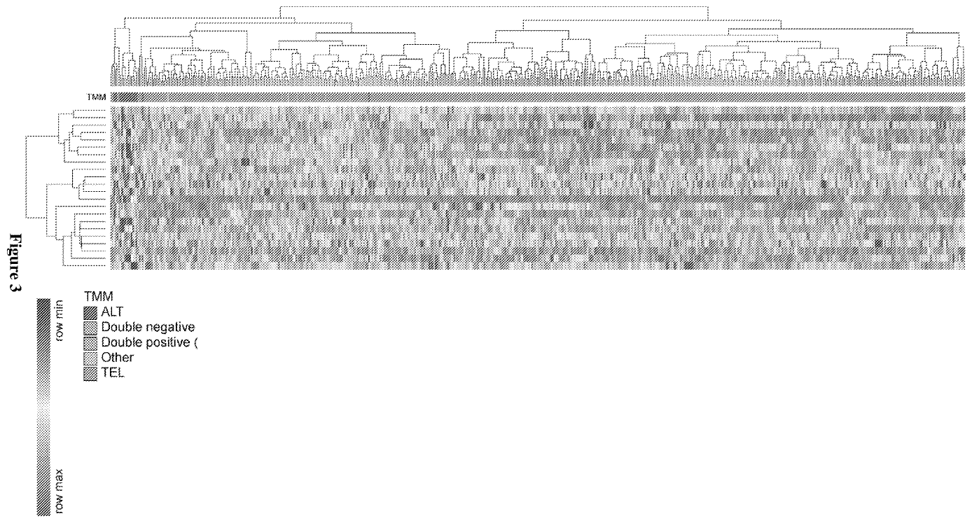

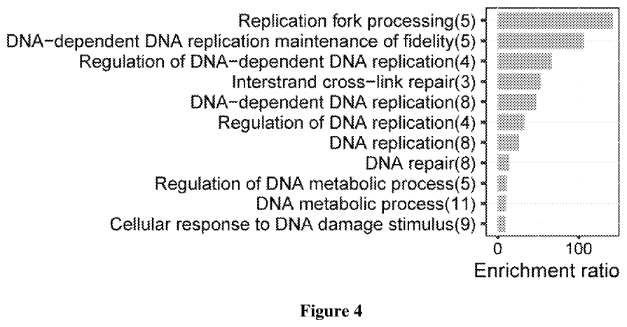

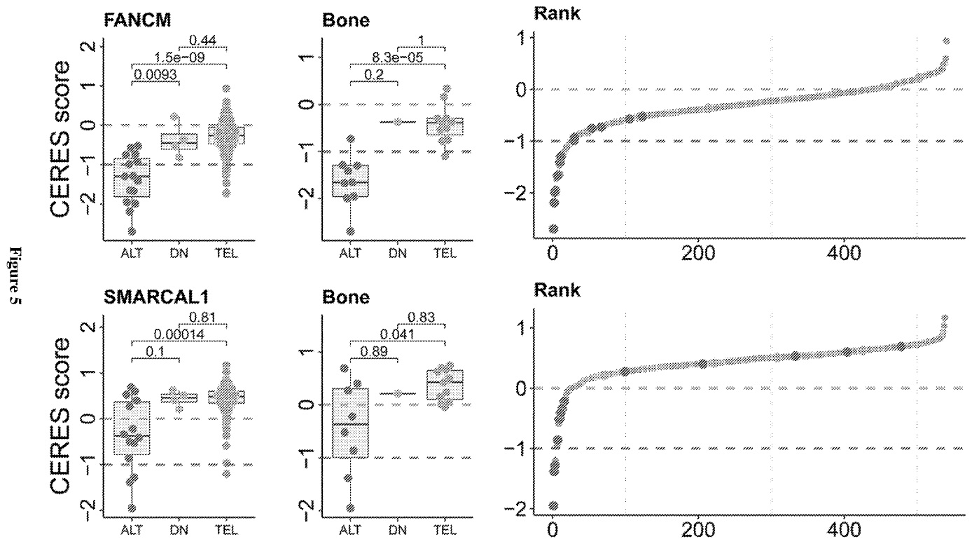

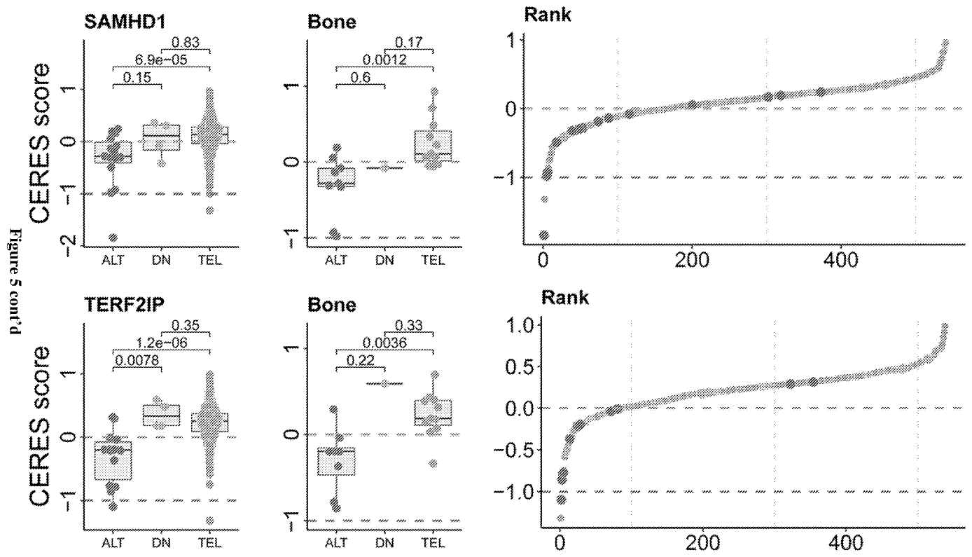

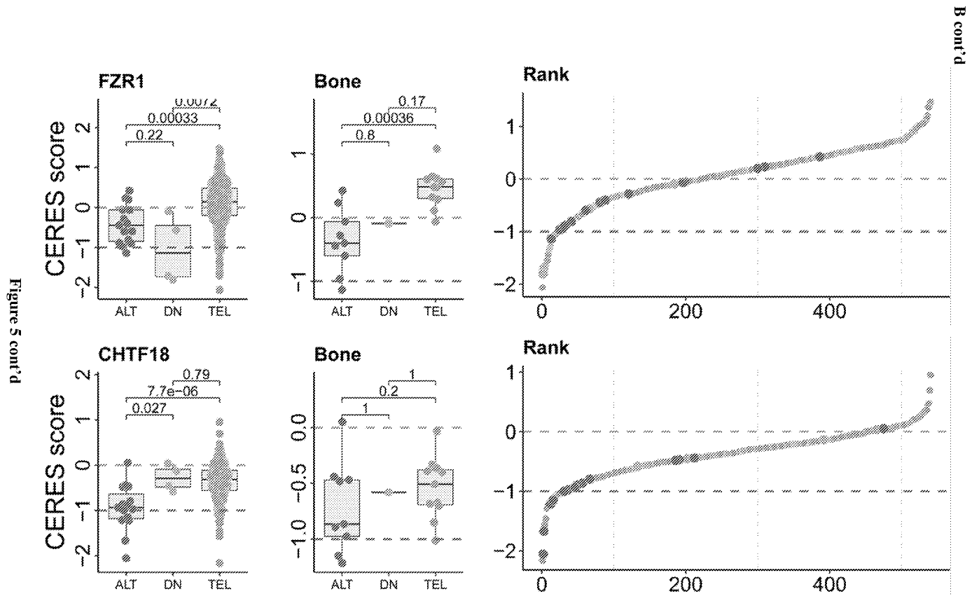

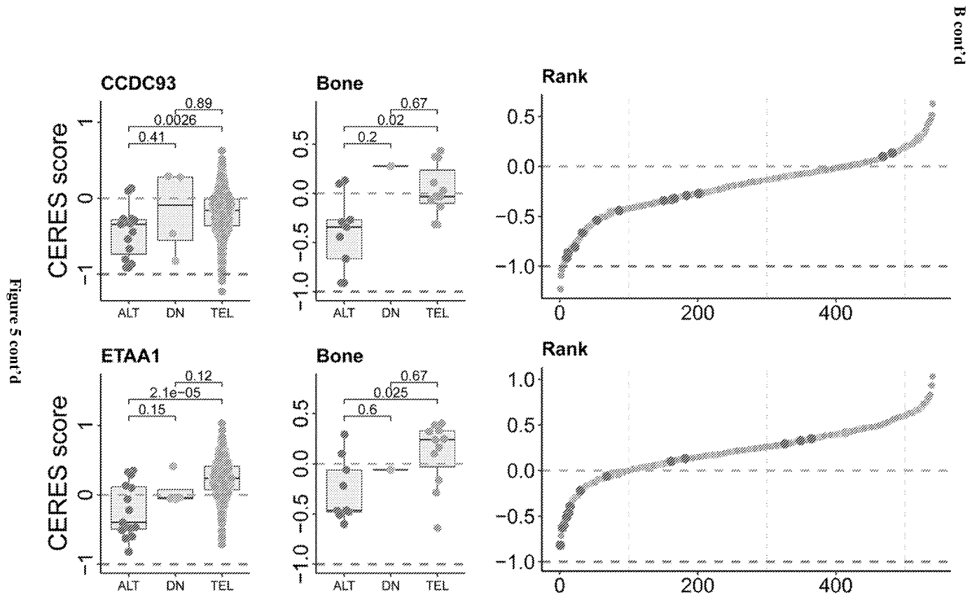

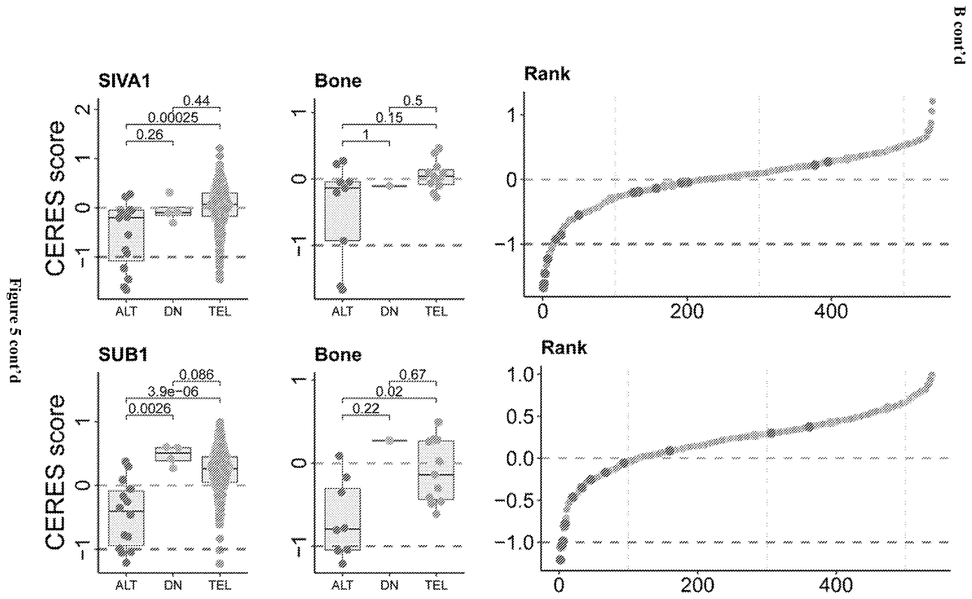

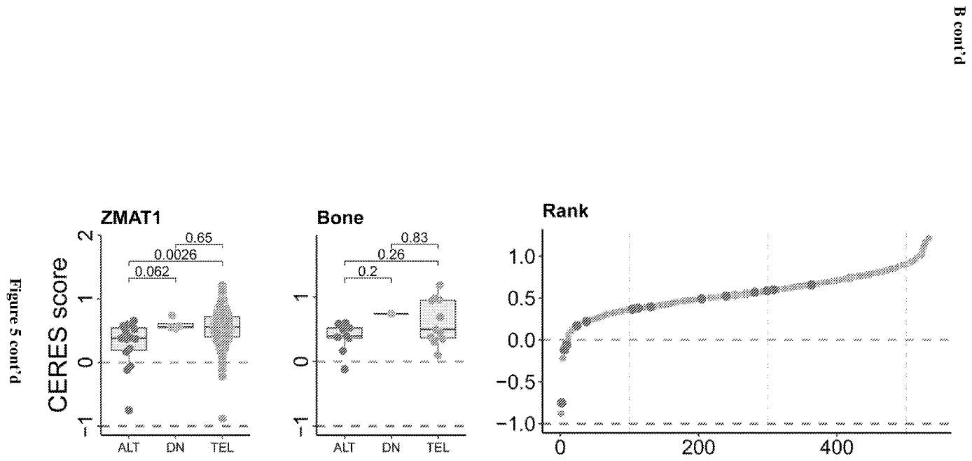

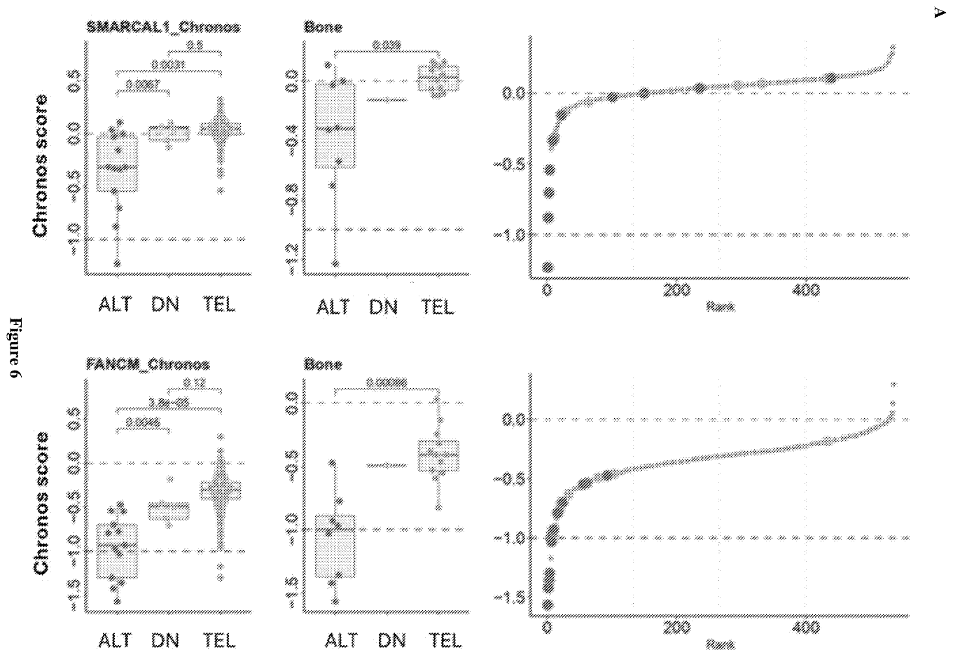

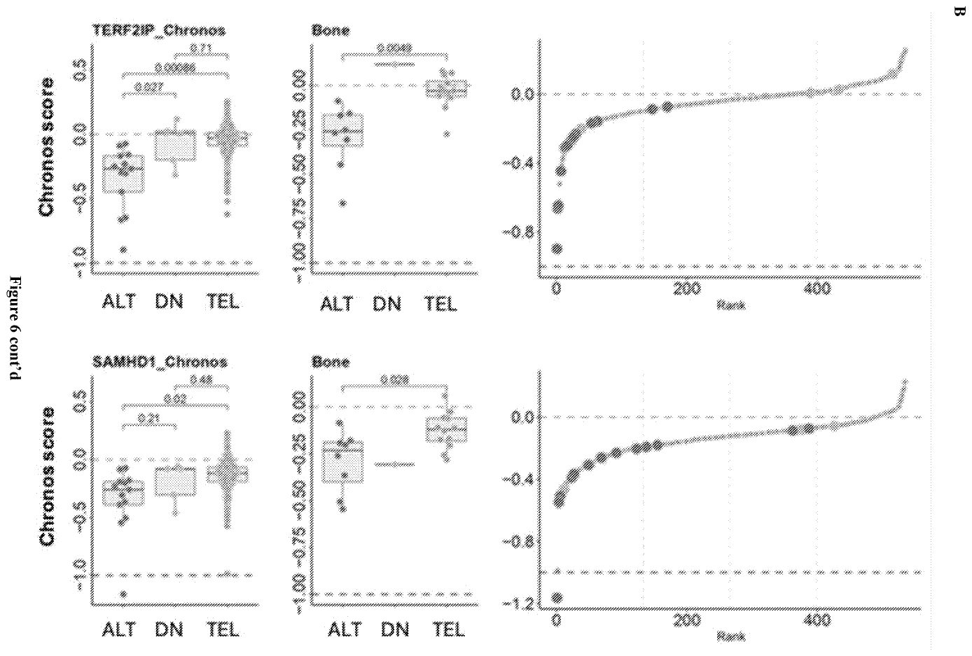

The present analyses identified a set of 18 genes as ALT cancer dependencies, including 6 genes previously shown to be involved in an ALT mechanism in independent molecular biology experiments. The identification of known ALT target genes in the analysis verifies the utility and validity of the bioinformatic approach taken. Of the remaining 12 novel genes identified in the analyses, the 8 genes with highest statistical significance have been further investigated as potential therapeutic targets in ALT cancers; TERF2IP, SAMHD1, CCDC93, CHTF18, ETAA1, ZMAT1, SIVA1, FZR1 and SUB1 (FIGS. 1-6).

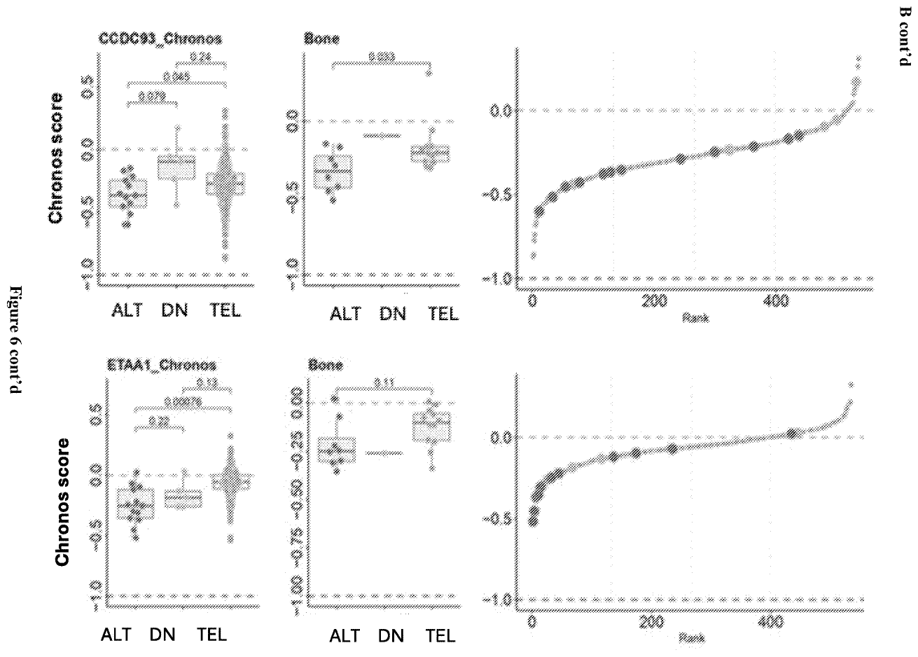

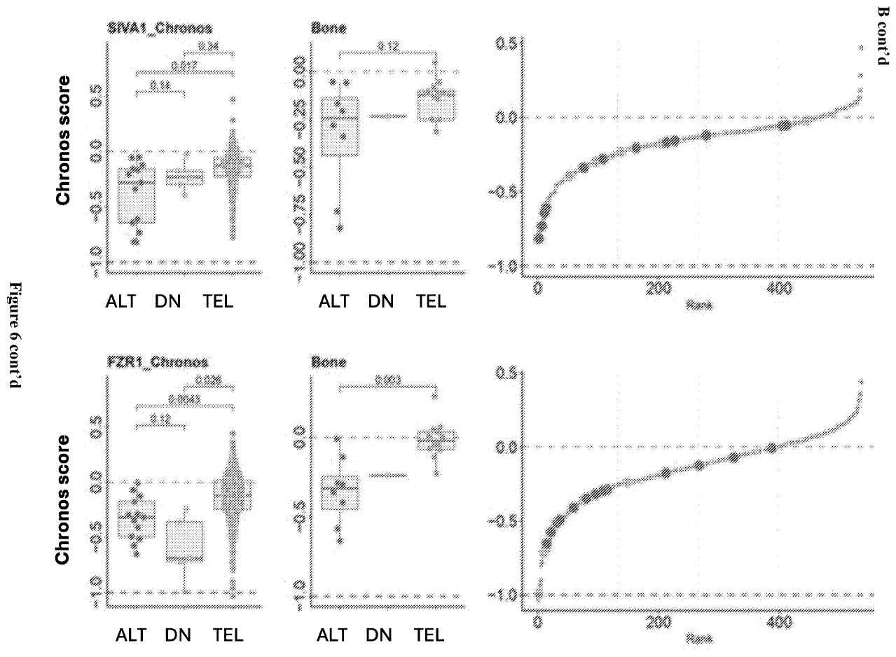

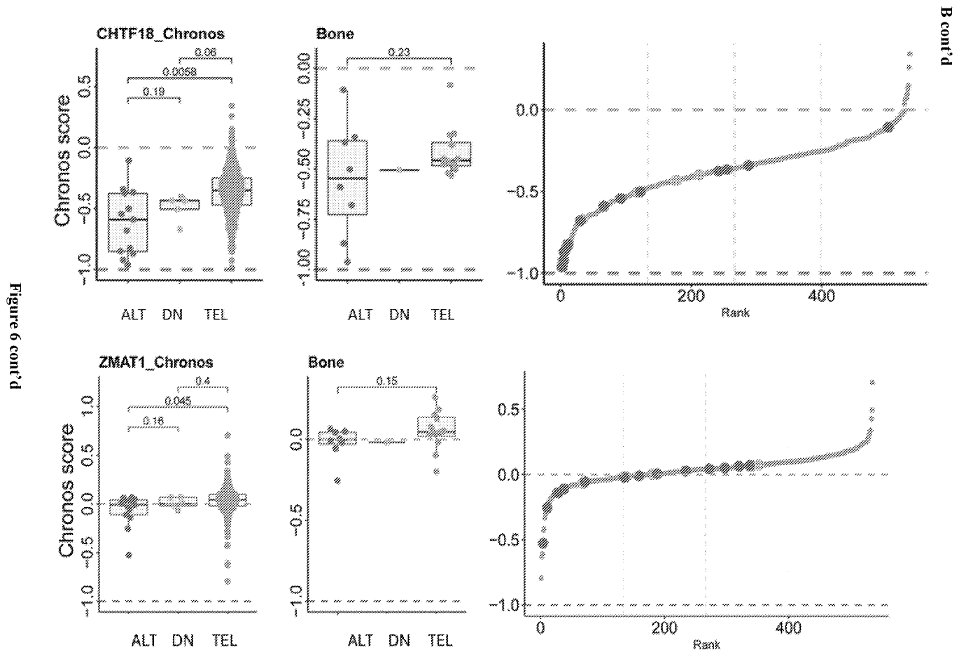

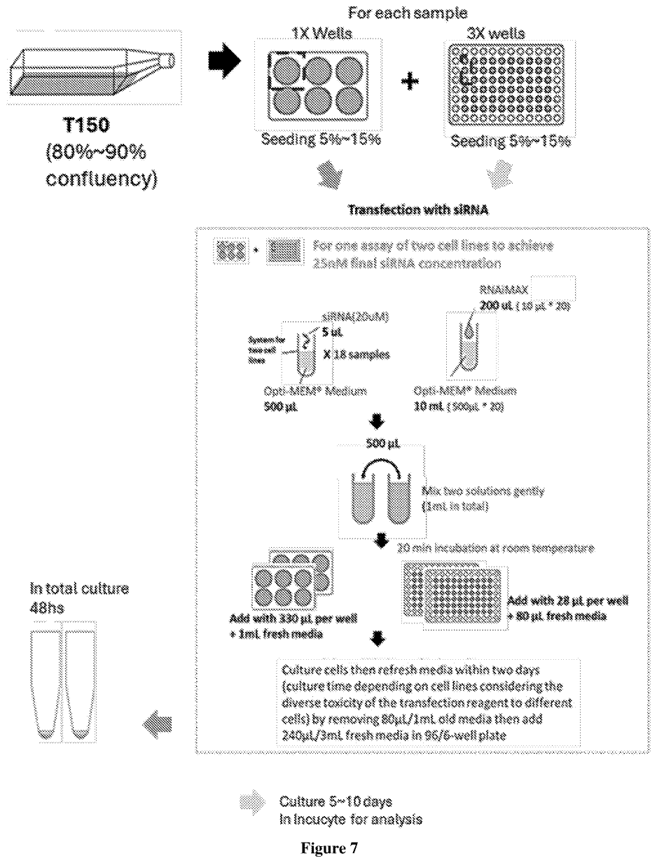

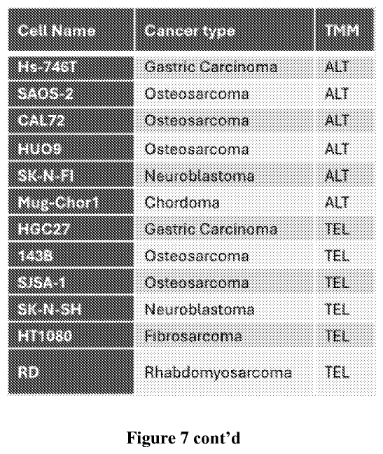

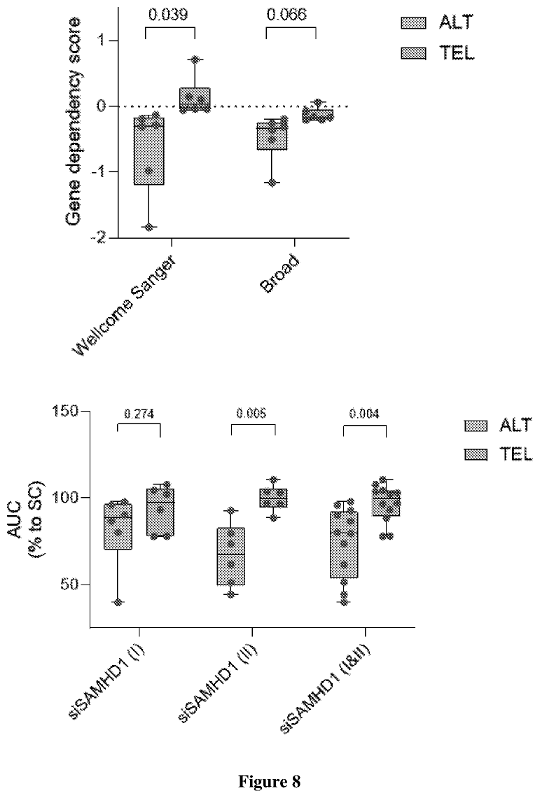

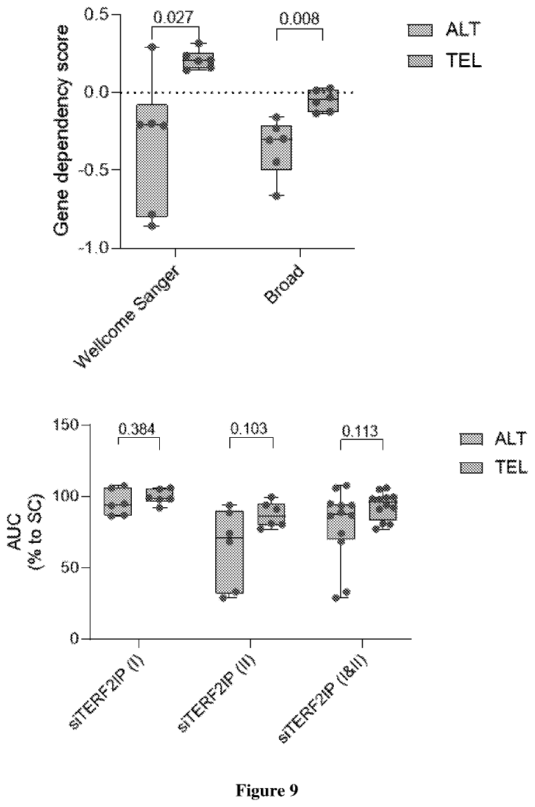

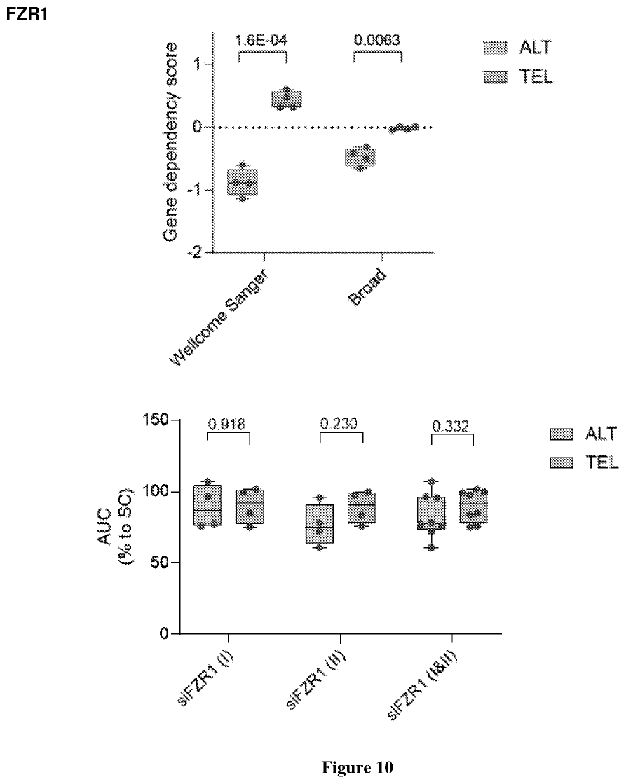

Cell biology assays were conducted whereby the expression of target genes was suppressed in 8-12 representative cell lines using siRNA. Multiple (2-4) siRNAs were tested for each gene. Following introduction of siRNA, the cells were monitored for survival and growth over 5-10 days (FIG. 7). These experiments confirmed that suppression of SAMHD1, TERF2IP and FZR1 expression inhibits proliferation of ALT cell lines (FIGS. 8-10).

### REFERENCES

1. Henson J D, Cao Y, Huschtscha L I, Chang A C, Au A Y, Pickett H A, et al. DNA C-circles are specific and quantifiable markers of alternative-lengthening-of-telomeres activity. Nat Biotechnol. 2009; 27 (12): 1181-5.
2. Yeager T R, Neumann A A, Englezou A, Huschtscha L I, Noble J R, Reddel R R.

Telomerase-negative immortalized human cells contain a novel type of promyelocytic leukemia (PML) body. Cancer Res. 1999; 59 (17): 4175-9.
3. Dahlgren P N, Bishop K, Dey S, Herbert B S, Tanaka H. Development of a New Monochrome Multiplex qPCR Method for Relative Telomere Length Measurement in Cancer. Neoplasia. 2018; 20 (5): 425-31.
4. Tanaka H, Beam M J, Caruana K. The presence of telomere fusion in sporadic colon cancer independently of disease stage, TP53/KRAS mutation status, mean telomere length, and telomerase activity. Neoplasia. 2014; 16 (10): 814-23.
5. Vaziri H, Dragowska W, Allsopp R C, Thomas T E, Harley C B, Lansdorp P M. Evidence for a mitotic clock in human hematopoietic stem cells: loss of telomeric DNA with age. Proc Natl Acad Sci USA. 1994; 91 (21):9857-60.
6. Frank L, Rademacher A, Mucke N, Tirier S M, Koeleman E, Knotz C, et al. ALT-FISH quantifies alternative lengthening of telomeres activity by imaging of single-stranded repeats. Nucleic Acids Res. 2022; 50 (11): e61.
7. Smyth G K. Linear models and empirical bayes methods for assessing differential expression in microarray experiments. Stat Appl Genet Mol Biol. 2004; 3: Article3.
8. Benjamini Y, Hochberg Y. Controlling the false discovery rate: a practical and powerful approach to multiple testing. Journal of the Royal statistical society: series B (Methodological). 1995; 57 (1): 289-300.

1. A method of treating, preventing or ameliorating an ALT-dependent cancer in a subject, said method including the step of administering to the subject a therapeutically effective amount of an agent that reduces an expression and/or an activity of one or more of TERF2IP, SAMHD1, CCDC93, CHTF18, ETAA1, ZMAT1, SIVA1, FZR1 and SUB1 to thereby treat, prevent or ameliorate the ALT-dependent cancer in the subject.

2. The method of claim 1, further including the earlier or initial step of identifying the ALT-dependent cancer in the subject.

3. The method of claim 1, further including the step of administering a therapeutically effective amount of a further anti-cancer agent to the subject.

4. A method of determining whether a cancer of a subject is responsive to or suitable for treatment with an agent that reduces an expression and/or an activity of one or more of TERF2IP, SAMHD1, CCDC93, CHTF18, ETAA1, ZMAT1, SIVA1, FZR1 and SUB1, said method including the step of determining an ALT status for the cancer of the subject, wherein a positive ALT status is indicative of the subject's cancer being responsive to or suitable for treatment with the agent.

5. The method of claim 4, including the further step of administering a therapeutically effective amount of the agent to the subject if the ALT status of the subject's cancer is positive.

6. The method of claim 1, wherein the cancer or ALT-dependent cancer is an osteosarcoma, a neuroblastoma, a gastric cancer, a liposarcoma, a glioblastoma, an astrocytoma or a bladder carcinoma.

7. A method of reducing the viability and/or growth of an ALT-dependent cancer cell, said method including the step of contacting the ALT-dependent cancer cell with an effective amount of an agent that reduces an expression and/or an activity of one or more of TERF2IP, SAMHD1, CCDC93, CHTF18, ETAA1, ZMAT1, SIVA1, FZR1 and SUB1.

8. The method of claim 7, wherein the ALT-dependent cancer cell is an osteosarcoma cell, a neuroblastoma cell, a gastric cancer cell, a liposarcoma cell, a glioblastoma cell, an astrocytoma cell or a bladder carcinoma cell.

9. The method of claim 1, wherein the agent is selected from the group consisting of a genetic inhibitor, a targeted nuclease, a small molecule, a peptide, a protein, an antibody or fragment thereof and any combination thereof.

10. The method of claim 9, wherein the genetic inhibitor is selected from the group consisting of siRNA, shRNA, antisense oligonucleotides, an aptamer, a targeted nuclease and any combination thereof.

11. The method of claim 9, wherein the agent is or comprises HIV-2/SIV viral protein x (Vpx) or a derivative thereof.

12.-13. (canceled)

14. A method for identifying or producing an agent for use in the prevention, amelioration or treatment of an ALT-dependent cancer in a subject, said method including the steps of:
(a) contacting a cell that expresses one or more of TERF2IP, SAMHD1, CCDC93, CHTF18, ETAA1, ZMAT1, SIVA1, FZR1 and SUB1 with a candidate agent; and
(b) determining whether the candidate agent modulates an expression and/or an activity of one or more of TERF2IP, SAMHD1, CCDC93, CHTF18, ETAA1, ZMAT1, SIVA1, FZR1 and SUB1.

15. The method of claim 14, wherein the candidate agent, at least partly, reduces, eliminates, suppresses or inhibits the expression and/or the activity of one or more of TERF2IP, SAMHD1, CCDC93, CHTF18, ETAA1, ZMAT1, SIVA1, FZR1 and SUB1.

16. The method of claim 14, further including the step of determining whether the candidate agent modulates the viability and/or growth of the cell.

17. The method of claim 16, wherein the candidate agent, at least partly, reduces the viability and/or growth of the cell.

18. The method of claim 14, further including the step of determining whether the candidate agent modulates a level of genomic stability at one or more telomeres in the cell and/or a level of ALT activity in the cell.

19. The method of claim 18, wherein the candidate agent, at least partly, increases the level of genomic stability at one or more telomeres in the cell and/or at least partly, reduces the level of ALT activity in the cell.

20. The method of claim 14, wherein the cell is or comprises an ALT-dependent cancer cell.

21. The method of claim 20, wherein the ALT-dependent cancer cell is an osteosarcoma cell, a neuroblastoma cell, a gastric cancer cell, a liposarcoma cell, a glioblastoma cell, an astrocytoma cell or a bladder carcinoma cell.

22. The method of claim 14, further including one or more of the steps of:
selecting the candidate agent that modulates the expression and/or the activity of one or more of TERF2IP, SAMHD1, CCDC93, CHTF18, ETAA1, ZMAT1, SIVA1, FZR1 and SUB1;
isolating or purifying the candidate agent;
formulating the candidate agent into a pharmaceutical formulation; and
adding the candidate agent or the pharmaceutical formulation to packaging and/or a container.

23. (canceled)
